SEQUENCE LISTING (102 sequences)

SEQ ID NO: 1 - protein, 120 aa, synthetic construct
EVQLVESGGGLVQPGGSLRLSCAASGFTFSSYAMHWVRQAPGKGLEWVAVISYDGSNKYYADSVKGRFTISRDNSKNTLYLQMNSLRAEDTAVYYCAKEGDSSRWSYDLWGRGTLVTVSS

SEQ ID NO: 2 - protein, 120 aa, synthetic construct
EVQLVESGGGLVQPGGSLRLSCAASGFTFSAYAMHWVRQAPGKGLEWVAVISYEGYNAYYADSVKGRFTISRDNSKNTLYLQMNSLRAEDTAVYYCAKEGDYSRWAYDYWGRGTLVTVSS

SEQ ID NO: 3 - protein, 120 aa, synthetic construct
EVQLVESGGGLVQPGGSLRLSCAASGFTFSAYAMHWVRQAPGKGLEWVAVISYEGYNAYYADSVKGRFTISRDNSKNTLYLQMNSLRAEDTAVYYCAKEGDYSRWAYDLWGRGTLVTVSS

SEQ ID NO: 4 - protein, 120 aa, synthetic construct
QVQLVESGGGVVQPGGSLRLSCAASGFTFSSYAMHWVRQAPGKGLEWVAVISYDGSNKYYADSVKGRFTISRDNSKNTLYLQMNSLRAEDTAVYYCAKEGDSSRWSYDLWGRGTLVTVSS

SEQ ID NO: 5 - protein, 120 aa, synthetic construct
QVQLVESGGGVVQPGGSLRLSCAASGFTFSSYDMHWVRQAPGKGLEWVAVISYDGSNKYYADSVKGRFTISRDNSKNTLYLQMNSLRAEDTAVYYCAKEGDSSRWSYDLWGRGTLVTVSS

SEQ ID NO: 6 - protein, 120 aa, synthetic construct
QVQLVESGGGVVQPGGSLRLSCAASGFAFSSYDMHWVRQAPGKGLEWVAVISYDGSNKYYADSVKGRFTISRDNSKNTLYLQMNSLRAEDTAVYYCAKEGDSSRWSYDLWGRGTLVTVSS

SEQ ID NO: 7 - protein, 120 aa, synthetic construct
QVQLVESGGGVVQPGGSLRLSCAASGFTFSSYAMHWVRQAPGKGLEWVAVISYDGSNKYYADSVKGRFTISRDNSKNTLYLQMNSLRAEDTAVYYCAKEGDSSRWGYDLWGRGTLVTVSS

SEQ ID NO: 8 - protein, 120 aa, synthetic construct
QVQLVESGGGVVQPGGSLRLSCAASGFTFSSYAMHWVRQAPGKGLEWVAVVSYDGSNKYYADSVKGRFTISRDNSKNTLYLQMNSLRAEDTAVYYCAKEGDSSRWSYDLWGRGTLVTVSS

SEQ ID NO: 9 - protein, 120 aa, synthetic construct
QVQLVESGGGVVQPGGSLRLSCAASGFTFSSYAMHWVRQAPGKGLEWVAVVSYDGSNKYYADSVKGRFTISRDNSKNTLYLQMNSLRAEDTAVYYCAKEGDSSRWGYDLWGRGTLVTVSS

SEQ ID NO: 10 - protein, 120 aa, synthetic construct
QVQLVESGGGVVQPGGSLRLSCAASGFAFSSYDMHWVRQAPGKGLEWVAVVSYDGSNKYYADSVKGRFTISRDNSKNTLYLQMNSLRAEDTAVYYCAKEGDSSRWGYDLWGRGTLVTVSS

SEQ ID NO: 11 - protein, 120 aa, synthetic construct
QVQLVESGGGVVQPGGSLRLSCAASGFTFSSYDMHWVRQAPGKGLEWVAVVSYDGSNKYYADSVKGRFTISRDNSKNTLYLQMNSLRAEDTAVYYCAKEGDSSRWGYDLWGRGTLVTVSS

SEQ ID NO: 12 - protein, 111 aa, synthetic construct
QSALTQPASVSGSPGQSITISCTGTSSDVGGYNYVSWYQQHPGKAPKLMIYDVTNRPSGVSNRFSGSKSGNTASLTISGLQAEDEADYYCSSYTIASTLVVFGGGTKLTVL

SEQ ID NO: 13 - protein, 111 aa, synthetic construct
QSALTQPASVSGSPGQSITISCTGTSSDVGGYNYVSWYQQHPGKAPKLMIYDVTNYPSGVSNRFSGSKSGNTASLTISGLQAEDEADYYCSSYTVATTLVVFGGGTKLTVL

SEQ ID NO: 14 - protein, 111 aa, synthetic construct
QSALTQPASVSGSPGQSITISCTGTSSDVGGYNYVSWYQQHPGKAPKLMIYDVTNRPSGVSNRFSGSKSGNTASLTISGLQAEDEADYYCSSYTIASTPVVFGGGTKLTVL

SEQ ID NO: 15 - protein, 111 aa, synthetic construct
QSALTQPASVSGSPGQSITISCTGTSSDVGGYNYVSWYQQHPGKAPKLMIYDVTNRPSGVSNRFSGSKSGNTASLTISGLQAEDEADYYCSSYTIASTFVVFGGGTKLTVL

SEQ ID NO: 16 - protein, 111 aa, synthetic construct
QSALTQPASVSGSPGQSITISCTGTSSDVGGYNYVSWYQQHPGKAPKLMIYDVTNRPSGVSNRFSGSKSGNTASLTISGLQAEDEADYYCSSYAIASTLVVFGGGTKLTVL

SEQ ID NO: 17 - protein, 111 aa, synthetic construct
QSALTQPASVSGSPGQSITISCTGTSSDVGGYNYVSWYQQHPGKAPKLMIYDVTDRPSGVSNRFSGSKSGNTASLTISGLQAEDEADYYCSSYTIASTLVVFGGGTKLTVL

SEQ ID NO: 18 - protein, 111 aa, synthetic construct
QSALTQPASVSGSPGQSITISCTGTSSDVGGYNYVSWYQQHPGKAPKLMIYDVTDRPSGVSNRFSGSKSGNTASLTISGLQAEDEADYYCSSYAIASTLVVFGGGTKLTVL

SEQ ID NO: 19 - protein, 111 aa, synthetic construct
QSALTQPASVSGSPGQSITISCTGTSSDVGGYNYVSWYQQHPGKAPKLMIYDVTDRPSGVSNRFSGSKSGNTASLTISGLQAEDEADYYCSSYAIASTPVVFGGGTKLTVL

SEQ ID NO: 20 - protein, 13 aa, synthetic construct
AASGFTFSSYAMH

SEQ ID NO: 21 - protein, 10 aa, synthetic construct
VISYDGSNKY

SEQ ID NO: 22 - protein, 13 aa, synthetic construct
AKEGDSSRWSYDL

SEQ ID NO: 23 - protein, 13 aa, synthetic construct
AASGFTFSAYAMH

SEQ ID NO: 24 - protein, 10 aa, synthetic construct
VISYEGYNAY

SEQ ID NO: 25 - protein, 13 aa, synthetic construct
AKEGDYSRWAYDY

SEQ ID NO: 26 - protein, 13 aa, synthetic construct
AASGFTFSAYAMH

SEQ ID NO: 27 - protein, 10 aa, synthetic construct
VISYEGYNAY

SEQ ID NO: 28 - protein, 13 aa, synthetic construct
AKEGDYSRWAYDL

SEQ ID NO: 29 - protein, 13 aa, synthetic construct
AASGFTFSSYAMH

SEQ ID NO: 30 - protein, 10 aa, synthetic construct
VISYDGSNKY

SEQ ID NO: 31 - protein, 13 aa, synthetic construct
AKEGDSSRWSYDL

SEQ ID NO: 32 - protein, 13 aa, synthetic construct
AASGFTFSSYDMH

SEQ ID NO: 33 - protein, 10 aa, synthetic construct
VISYDGSNKY

SEQ ID NO: 34 - protein, 13 aa, synthetic construct
AKEGDSSRWSYDL

SEQ ID NO: 35 - protein, 13 aa, synthetic construct
AASGFAFSSYDMH

SEQ ID NO: 36 - protein, 10 aa, synthetic construct
VISYDGSNKY

SEQ ID NO: 37 - protein, 13 aa, synthetic construct
AKEGDSSRWSYDL

SEQ ID NO: 38 - protein, 13 aa, synthetic construct
AASGFTFSSYAMH

SEQ ID NO: 39 - protein, 10 aa, synthetic construct
VISYDGSNKY

SEQ ID NO: 40 - protein, 13 aa, synthetic construct
AKEGDSSRWGYDL

SEQ ID NO: 41 - protein, 13 aa, synthetic construct
AASGFTFSSYAMH

SEQ ID NO: 42 - protein, 10 aa, synthetic construct
VVSYDGSNKY

SEQ ID NO: 43 - protein, 13 aa, synthetic construct
AKEGDSSRWSYDL

SEQ ID NO: 44 - protein, 13 aa, synthetic construct
AASGFTFSSYAMH

SEQ ID NO: 45 - protein, 10 aa, synthetic construct
VVSYDGSNKY

SEQ ID NO: 46 - protein, 13 aa, synthetic construct
AKEGDSSRWGYDL

SEQ ID NO: 47 - protein, 13 aa, synthetic construct
AASGFAFSSYDMH

SEQ ID NO: 48 - protein, 10 aa, synthetic construct
VVSYDGSNKY

SEQ ID NO: 49 - protein, 13 aa, synthetic construct
AKEGDSSRWGYDL

SEQ ID NO: 50 - protein, 13 aa, synthetic construct
AASGFTFSSYDMH

SEQ ID NO: 51 - protein, 10 aa, synthetic construct
VVSYDGSNKY

SEQ ID NO: 52 - protein, 13 aa, synthetic construct
AKEGDSSRWGYDL

SEQ ID NO: 53 - protein, 14 aa, synthetic construct
TGTSSDVGGYNYVS

SEQ ID NO: 54 - protein, 8 aa, synthetic construct
YDVTNRPS

SEQ ID NO: 55 - protein, 11 aa, synthetic construct
SSYTIASTLVV

SEQ ID NO: 56 - protein, 14 aa, synthetic construct
TGTSSDVGGYNYVS

SEQ ID NO: 57 - protein, 8 aa, synthetic construct
YDVTNYPS

SEQ ID NO: 58 - protein, 11 aa, synthetic construct
SSYTVATTLVV

SEQ ID NO: 59 - protein, 14 aa, synthetic construct
TGTSSDVGGYNYVS

SEQ ID NO: 60 - protein, 8 aa, synthetic construct
YDVTNRPS

SEQ ID NO: 61 - protein, 11 aa, synthetic construct
SSYTIASTPVV

SEQ ID NO: 62 - protein, 14 aa, synthetic construct
TGTSSDVGGYNYVS

SEQ ID NO: 63 - protein, 8 aa, synthetic construct
YDVTNRPS

SEQ ID NO: 64 - protein, 11 aa, synthetic construct
SSYTIASTFVV

SEQ ID NO: 65 - protein, 14 aa, synthetic construct
TGTSSDVGGYNYVS

SEQ ID NO: 66 - protein, 8 aa, synthetic construct
YDVTNRPS

SEQ ID NO: 67 - protein, 11 aa, synthetic construct
SSYAIASTLVV

SEQ ID NO: 68 - protein, 14 aa, synthetic construct
TGTSSDVGGYNYVS

SEQ ID NO: 69 - protein, 8 aa, synthetic construct
YDVTDRPS

SEQ ID NO: 70 - protein, 11 aa, synthetic construct
SSYTIASTLVV

SEQ ID NO: 71 - protein, 14 aa, synthetic construct
TGTSSDVGGYNYVS

SEQ ID NO: 72 - protein, 8 aa, synthetic construct
YDVTDRPS

SEQ ID NO: 73 - protein, 11 aa, synthetic construct
SSYAIASTLVV

SEQ ID NO: 74 - protein, 14 aa, synthetic construct
TGTSSDVGGYNYVS

SEQ ID NO: 75 - protein, 8 aa, synthetic construct
YDVTDRPS

SEQ ID NO: 76 - protein, 11 aa, synthetic construct
SSYAIASTPVV

SEQ ID NO: 77 - protein, 125 aa, synthetic construct
EVQLVESGGGLVQPGGSLRLSCAASGFTFNTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 78 - protein, 110 aa, synthetic construct
DIQMTQSPSSLSASVGDRVTITCRSSTGAVTTSNYANWVQQKPGKAPKGLIGGTNKRAPGVPSRFSGSLIGDKATLTISSLQPEDFATYYCALWYSNLWVFGQGTKVEIK

SEQ ID NO: 79 - protein, 12 aa, synthetic construct
ASGFTFNTYAMN

SEQ ID NO: 80 - protein, 8 aa, synthetic construct
RIRSKYNN

SEQ ID NO: 81 - protein, 16 aa, synthetic construct
VRHGNFGNSYVSWFAY

SEQ ID NO: 82 - protein, 13 aa, synthetic construct
SSTGAVTTSNYAN

SEQ ID NO: 83 - protein, 8 aa, synthetic construct
GGTNKRAP

SEQ ID NO: 84 - protein, 9 aa, synthetic construct
ALWYSNLWV

SEQ ID NO: 85 - protein, 344 aa, synthetic construct
EVQLVESGGGLVQPGGSLRLSCAASGFTFNTYAMNWVRQAPGKGLEWVGRIRSKYNNYATYYADSVKDRFTISRDDSKNSLYLQMNSLTACDTAVYYCVRHGNFGNSYVSWFAYWGQGTLVTVSSGGGGSQSALTQPASVSGSPGQSITISCTGTSSDVGGYNYVSWYQQHPGKAPKLMIYDVTYRPSGVSNRFSGSKSGNTASLTISGLQAEDEADYYCSSYTVATTLVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 86 - protein, 342 aa, synthetic construct
EVQLVESGGGLVQPGGSLRLSCAASGFTFSAYAMHWVRQAPGKGLEWVAVISYEGYNAYYADSVKGRFTISRDNSKNTLYLQMNSLTACDTAVYYCAKEGDYSRWAYDYWGRGTLVTVSSGGGGSDIQMTQSPSSLSASVGDRVTITCRSSTGAVTTSNYANWVQQKPGKAPKGLIGGTNKRAPGVPSRFSGSLIGDKATLTISSLQPEDFATYYCALWYSNLWVFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 344 aa, synthetic construct
EVQLVESGGGLVQPGGSLRLSCAASGFTFNTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGGGSQSALTQPASVSGSPGQSITISCTGTSSDVGGYNYVSWYQQHPGKAPKLMIYDVTYRPSGVSNRFSGSKSGNTASLTISGLQAEDEADYYCSSYTVATTLVVFGGGTKLTVLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 88 - protein, 361 aa, synthetic construct
MGWSCIILFLVATATGVHSEVQLVESGGGLVQPGGSLRLSCAASGFTFSAYAMHWVRQAPGKGLEWVAVISYEGYNAYYADSVKGRFTISRDNSKNTLYLQMNSLRAEDTAVYYCAKEGDYSRWAYDYWGRGTLVTVSSGGGGSDIQMTQSPSSLSASVGDRVTITCRSSTGAVTTSNYANWVQQKPGKAPKGLIGGTNKRAPGVPSRFSGSLIGDKATLTISSLQPEDFATYYCALWYSNLWVFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 15 aa, synthetic construct
GGGGSGGGGSGGGGS

SEQ ID NO: 90 - protein, 10 aa, synthetic construct
GGGGSGGGGS

SEQ ID NO: 91 - protein, 5 aa, synthetic construct
GGGGS

SEQ ID NO: 92 - protein, 20 aa, synthetic construct
GSGGGGSGGGGSGGSGGGGS

SEQ ID NO: 93 - protein, 6 aa, synthetic construct
SGGGGS

SEQ ID NO: 94 - protein, 4 aa, synthetic construct
GGGS

SEQ ID NO: 95 - protein, 4 aa, synthetic construct
VPGV

SEQ ID NO: 96 - protein, 5 aa, synthetic construct
VPGVG

SEQ ID NO: 97 - protein, 6 aa, synthetic construct
GVPGVG

SEQ ID NO: 98 - protein, 8 aa, synthetic construct
GVGVPGVG

SEQ ID NO: 99 - protein, 10 aa, synthetic construct
VPGVGVPGVG

SEQ ID NO: 100 - protein, 7 aa, synthetic construct
GGSSRSS

SEQ ID NO: 101 - protein, 18 aa, synthetic construct
GGSSRSSSSGGGGSGGGG

SEQ ID NO: 102 - protein, 6 aa, synthetic construct
HHHHHH

(19) United States
(12) Patent Application Publication
(10) Pub. No.: US 2025/0145733 A1
(43) Pub. Date: May 8, 2025

(54) TUMOR-SPECIFIC BISPECIFIC IMMUNE CELL ENGAGER

(71) Applicant: The Regents of the University of California, Oakland, CA (US)

(72) Inventors: Bin Liu, San Francisco, CA (US); Scott Bidlingmaier, San Francisco, CA (US); Yang Su, South San Francisco, CA (US)

(73) Assignee: The Regents of the University of California, Oakland, CA (US)

(21) Appl. No.: 18/693,921

(22) PCT Filed: Sep. 21, 2022
(86) PCT No.: PCT/US2022/044205

§ 371 Date: Mar. 20, 2024

Related U.S. Application Data
(60) Provisional application No. 63/247,014, filed on Sep. 22, 2021.

(51) Int. Cl. C07K 16/40; C07K 16/28; G01N 33/574

(52) U.S. Cl. CPC C07K 16/40; C07K 16/2809; C12Y 301/03001; G01N 33/574; C07K 2317/31; C07K 2317/73; C07K 2317/92

(57) ABSTRACT

Disclosed herein are monospecific and bispecific antibodies that comprise a ALPPL2/ALPP-binding variable region. The bi-specific antibody is comprises of a monoclonal antibody targeting a tumor-specific cell surface antigen (ALPPL2/ALPP) with exquisite tissue specificity and a monoclonal antibody targeting a cell surface molecule (e.g., CD3) expressed by immune effector cells.

### CROSS-REFERENCE TO RELATED PATENT APPLICATIONS

The present patent application is a 371 U.S. National Phase application of International Application No. PCT/US2022/044205 filed Sep. 21, 2022 which claims benefit of priority to U.S. Provisional Patent Application No. 63/247,014, filed Sep. 22, 2021, which is incorporated by reference for all purposes.

### SEQUENCE LISTING

A Sequence Listing conforming to the rules of WIPO Standard ST.26 is hereby incorporated by reference. Said Sequence Listing has been filed as an electronic document via PatentCenter encloded as XML in UTF-8 text. The electronic document, created on Mar. 20, 2024, is entitled “081906=1345059-244210PC_SL.XML”, and is 93,087 bytes in size.

### BACKGROUND OF THE INVENTION

Identification of tumor-specific cell surface antigens has proven challenging, as the vast majority of tumor-associated antigens are also expressed in normal tissues. In our study of mesothelioma, we selected phage antibody display libraries on live tumor cells and tissues following counter-selection on normal cells and identified a panel of human antibodies that bind specifically to mesothelioma (An, F., et al. Mol Cancer Ther 7(3):569-78 (2008); Su, Y et al., Cancer Res., 2020 Aug. 31). One of the antibodies, M25, binds only to tumors but not any of the normal human tissues studied except for the placenta. We identified the tumor antigen bound by M25 as human alkaline phosphatase, placental-like 2 (ALPPL2, aka alkaline phosphatase, germ cell (ALPG)), a member of the human alkaline phosphatase family (Su et al, 2020). Among the four members of this family, ALPPL2 and placental alkaline phosphate (ALPP) are virtually identical in amino acid sequence (98% homology) and have a highly restricted normal tissue expression pattern, expressing in placental trophoblasts only. Both share high homology with the intestinal alkaline phosphatase (ALPI) (87% homology), and some homology with the tissue-nonspecific liver/bone/kidney phosphatase ALPL (57% homology). M25 binds specifically to ALPPL2 and ALPP but not ALPI or ALPL (Su et al, 2020). We performed immunohistochemistry (IHC) studies and showed that ALPPL2 is expressed in mesothelioma (Su et al, 2020) and a few other tumors such as seminoma, ovarian, pancreatic, gastric and colorectal cancers (WO2017095823A1; Hyrenius-Wittsten, A., et al., Science Translational Medicine 2021 Apr. 28; 13(591)), but not any other normal tissue except for placental trophoblasts, thus demonstrating an exquisite tissue specificity. ALPPL2 is therefore one of those rare cell surface antigens that can be classified as being truly tumor specific. To evaluate ALPPL2 as a potential therapeutic target, we constructed antibody-drug conjugates (ADCs) by conjugating microtubule inhibitors to our anti-ALPPL2 human monoclonal antibody M25 and showed that M25 ADCs potently inhibited tumor cell proliferation in vitro and mesothelioma cell line xenograft growth in vivo (Su et al, 2020).

### BRIEF SUMMARY OF THE INVENTION

In some aspects, the disclosure provides an antibody comprising a variable region that specifically binds to ALPPL2 and ALPP. In some embodiments, the antibody comprises a heavy chain variable region and a light chain variable region, wherein complementarity determining region (CDR) 1, CDR2 and CDR3 of the heavy chain variable region are selected from the following sets:
SEQ ID NOs: 20, 21, and 22;
SEQ ID NOs: 23, 24, and 25;
SEQ ID NOs: 26, 27, and 28;
SEQ ID NOs: 29, 30, and 31;
SEQ ID NOs: 32, 33, and 34;
SEQ ID NOs: 35, 36, and 37;
SEQ ID NOs: 38, 39, and 40;
SEQ ID NOs: 41, 42, and 43;
SEQ ID NOs: 44, 45, and 46;
SEQ ID NOs: 47, 48, and 49; or
SEQ ID NOs: 50, 51, and 52;
CDR1, CDR2, and CDR3 of the light chain variable region are selected from the following sets:
SEQ ID NOs: 53, 54, and 55;
SEQ ID NOs: 56, 57, and 58;
SEQ ID NOs: 59, 60, and 61;
SEQ ID NOs: 62, 63, and 64;
SEQ ID NOs: 65, 66, and 67;
SEQ ID NOs: 68, 69, and 70;
SEQ ID NOs: 71, 72, and 73; or
SEQ ID NOs: 74, 75, and 76,
with the proviso that the antibody does not have the heavy chain variable region of M25FYIA and the light chain variable region of M25FYIA.

In some embodiments, CDR1, CDR2, and CDR3 of the heavy chain variable region comprise SEQ ID NO: 23, SEQ ID NO: 24, and SEQ ID NO: 25, respectively; and CDR1, CDR2, and CDR3 of the light chain variable region comprise SEQ ID NO: 56, SEQ ID NO: 57, and SEQ ID NO: 58, respectively; or
CDR1, CDR2, and CDR3 of the heavy chain variable region comprise SEQ ID NO: 26, SEQ ID NO: 27, and SEQ ID NO: 28, respectively; and CDR1, CDR2, and CDR3 of the light chain variable region comprise SEQ ID NO: 56, SEQ ID NO: 57, and SEQ ID NO: 58, respectively; or
CDR1, CDR2, and CDR3 of the heavy chain variable region comprise SEQ ID NO: 44, SEQ ID NO: 45, and SEQ ID NO: 46, respectively; and CDR1, CDR2, and CDR3 of the light chain variable region comprise SEQ ID NO: 71, SEQ ID NO: 72, and SEQ ID NO: 73, respectively; or
CDR1, CDR2, and CDR3 of the heavy chain variable region comprise SEQ ID NO: 38, SEQ ID NO: 39, and SEQ ID NO: 40, respectively; and CDR1, CDR2, and CDR3 of the light chain variable region comprise SEQ ID NO: 65, SEQ ID NO: 66, and SEQ ID NO: 67, respectively; or
CDR1, CDR2, and CDR3 of the heavy chain variable region comprise SEQ ID NO: 41, SEQ ID NO: 42, and SEQ ID NO: 43, respectively; and CDR1, CDR2, and CDR3 of the light chain variable region comprise SEQ ID NO: 53, SEQ ID NO: 54, and SEQ ID NO: 55, respectively; or
CDR1, CDR2, and CDR3 of the heavy chain variable region comprise SEQ ID NO: 35, SEQ ID NO: 36, and SEQ ID NO: 37, respectively; and CDR1, CDR2, and CDR3 of the light chain variable region comprise SEQ ID NO: 59, SEQ ID NO: 60, and SEQ ID NO: 61, respectively.

In some embodiments, the heavy chain variable region is selected from the group consisting of SEQ ID NO: 1, 2, 3, 4, 5, 6, 7, 8, 9, 10 and 11. In some embodiments, the light chain variable region is selected from the group consisting of SEQ ID NO: 12, 13, 14, 15, 16, 17, 18, and 19.

In some embodiments, the heavy chain variable region comprises SEQ ID NO: 1; and the light chain variable region comprises SEQ ID NO: 12; or
the heavy chain variable region comprises SEQ ID NO: 2; and the light chain variable region comprises SEQ ID NO: 13; or
the heavy chain variable region comprises SEQ ID NO: 3; and the light chain variable region comprises SEQ ID NO: 13; or
the heavy chain variable region comprises SEQ ID NO: 9; and the light chain variable region comprises SEQ ID NO: 18; or
the heavy chain variable region comprises SEQ ID NO: 1; and the light chain variable region comprises SEQ ID NO: 12; or
the heavy chain variable region comprises SEQ ID NO: 7; and the light chain variable region comprises SEQ ID NO: 16; or
the heavy chain variable region comprises SEQ ID NO: 8; and the light chain variable region comprises SEQ ID NO: 12; or
the heavy chain variable region comprises SEQ ID NO: 6; and the light chain variable region comprises SEQ ID NO: 14.

In some embodiments, the antibody is an IgG, IgA or IgE antibody. In some embodiments, the antibody is an IgG antibody. In some embodiments, the antibody is an IgG1, IgG2, IgG3, or IgG4 antibody.

In some embodiments, the antibody is a monospecific antibody. In some embodiments, the antibody is linked to a cytotoxic agent. In some embodiments, the cytotoxic agent is a radionucleotide.

In some embodiments, the antibody is a bi-specific antibody comprising a second variable region that specifically binds to a second target protein, wherein the antibody comprises a second heavy chain variable region and a second light chain variable region. In some embodiments, the second target protein is expressed on the surface of a human immune effector cell. In some embodiments, the second target protein is human CD3. In some embodiments, the CDR1, CDR2, and CDR3 of the second heavy chain variable region comprise SEQ ID NO: 79, SEQ ID NO: 80 and SEQ ID NO: 81 and the CDR1, CDR2, and CDR3 of the second light chain variable region comprise SEQ ID NO: 82, SEQ ID NO: 83 and SEQ ID NO: 84. In some embodiments, the second heavy chain variable region comprises SEQ ID NO: 77 and the second light chain variable region comprises SEQ ID NO:78. In some embodiments, the bi-specific antibody comprises SEQ ID NO:85 and SEQ ID NO:86. In some embodiments, the bi-specific antibody comprises SEQ ID NO:87 and SEQ ID NO:88.

Also provided is a pharmaceutical composition comprising any antibody as described above or elsewhere herein.

Also provided is a nucleic acid encoding any antibody as described above or elsewhere herein.

Also provided is a vector comprising the nucleic acid sequence as described above or elsewhere herein.

Also provided is a cell comprising the nucleic acid as described above or elsewhere herein or the vector as described above or elsewhere herein. In some embodiments, the cell is a mammalian cell.

Also provided is a method for producing an antibody, the method comprising culturing the cell as described above or elsewhere herein under conditions to allow for production of the antibody.

Also provided is a method of killing a cancer cell, the method comprising, contacting the antibody as described above or elsewhere herein to a cancer cell. In some embodiments, the antibody is a bi-specific antibody comprising a second variable region that specifically binds to a second target protein, wherein the second target protein is human CD3, wherein the antibody comprises a second heavy chain variable region and a second light chain variable region, and wherein the cancer cell is brought in proximity to a peripheral blood mononuclear cell (PBMC) expressing CD3 by binding of the antibody. In some embodiments, the PBMC is a T-cell. In some embodiments, the cancer cell is a mesothelioma cell, a testicular cancer cell, an endometrial cancer cell, a pancreatic cancer cell, an ovarian cancer cell, a non-small cell lung cancer cell, a gastric cancer cell, or a colon cancer cell.

In some embodiments, the CDR1, CDR2, and CDR3 of the second heavy chain variable region comprise SEQ ID NO: 79, SEQ ID NO: 80 and SEQ ID NO: 81 and the CDR1, CDR2, and CDR3 of the second light chain variable region comprise SEQ ID NO: 82, SEQ ID NO: 83 and SEQ ID NO: 84. In some embodiments, the second heavy chain variable region comprises SEQ ID NO: 77 and the second light chain variable region comprises SEQ ID NO:78 In some embodiments, the antibody comprises SEQ ID NO:85 and SEQ ID NO: 86. In some embodiments, the antibody comprises SEQ ID NO:87 and SEQ ID NO:88.

In some embodiments, the antibody is linked to a cytotoxic agent. In some embodiments, the cytotoxic agent is a radionucleotide.

In some embodiments, the cancer cell is in a human having the cancer cell and the antibody is administered to the human, thereby killing the cancer cell.

Also provided is a chimeric antigen receptor (CAR)-expressing human cell, wherein the CAR comprises the heavy chain variable region and the light chain variable region as described above or elsewhere herein. In some embodiments, the human cell is a T-cell, natural killer cell or a macrophage.

Also provided is a method of detecting a tumor cell in a sample, the method comprising contacting an antibody as described above or elsewhere herein to the sample; and detecting specific binding of the antibody to the sample.

### BRIEF DESCRIPTION OF THE DRAWINGS

FIG. 1. Biolayer interferometry (BLI) measurements of the affinity of FYIA, FYIA_germ, FYIA_germ_6-6, and FYIA_germopt (aka SYLY) Fabs for human ALPPL2. Tips were loaded with Fabs, followed by an association step with 5 nM human ALPPL2-Fc then a dissociation step. Calculated affinities by curve fitting are 8.6 nM for FYIA, 13.0 nM for FYIA_germ, 0.88 nM for FYIA_germ_6-6, and 0.19 nM for FYIA_germopt.

FIG. 2. Binding of FYIA, FYIA_germ, and FYIA_germopt Fabs to living M28 cells. M28 cells were incubated with Fabs for 1 h at RT and binding was analyzed by flow cytometry. Calculated affinities by curve fitting are 30.55 nM for FYIA, 69.41 nM for FYIA_germ, and 0.97 nM for FYIA_germopt.

FIG. 3. Binding of FYIA, FYIA_germ, and FYIA_germopt Fabs to HEK293 cells stably transfected with human ALPI. HEK293-ALPI cells were incubated with Fabs for 1 h at RT and binding was analyzed by flow cytometry. No detectable binding was found. An ALPI-binding Fab, M25AD, was included in the study as a positive control.

FIG. 4. Additional Fabs (FYIA_germ_SY and FYIA_germ_6-6) were also studied along with FYIA, FYIA_germ, and FYIA_germopt Fabs for binding to living M28 cells. M28 cells were incubated with Fabs for 1 h at RT and binding was analyzed by flow cytometry. Calculated affinities by curve fitting are 1.27 nM for FYIA_germ_SY and 4.82 nM for FYIA_germ_6-6.

FIG. 5. Thermal-induced aggregation assay. In IgG1 form, FYIA has higher aggregation temperature (Tagg) than daratumumab (73-74° C. vs. 71-72° C.).

FIG. 6. Thermal-induced aggregation assay. SYLY Fab showed improved thermal stability with higher aggregation temperature (Tagg) than FYIA Fab (75-76° C. vs. 73° C.)

FIG. 7. Therapeutic antibody profiler analysis of M25. Scores calculated based on the five developability guidelines derived from clinical-stage therapeutic values all fall in the favorable range. PPC: patches of positive charge; PNC: patches of negative charge; SFvCSP: structural Fv charge symmetry parameter; PSH: patches of surface hydrophobicity.

FIG. 8. Therapeutic antibody profiler analysis of FYIA. Scores calculated based on the five developability guidelines derived from clinical-stage therapeutic values all fall in the favorable range. PPC: patches of positive charge; PNC: patches of negative charge; SFvCSP: structural Fv charge symmetry parameter; PSH: patches of surface hydrophobicity.

FIG. 9. Therapeutic antibody profiler analysis of SYLY. Scores calculated based on the five developability guidelines derived from clinical-stage therapeutic values all fall in the favorable range. PPC: patches of positive charge; PNC: patches of negative charge; SFvCSP: structural Fv charge symmetry parameter; PSH: patches of surface hydrophobicity.

FIG. 10A-10B: Summary of bispecific antibody forms. Adopted from Brinkmann U, Kontermann R E. The making of bispecific antibodies. mAbs 2017; 9:182-212.

FIG. 11. The SYLY-based ALPPL2×CD3 DSDbody was produced in HEK293A or ExpiCHO cells following transient transfection and purification by Ni-NTA. Purified DSDbody was analyzed on reducing SDS-PAGE. MW: molecular weight marker. The two chains migrate at a similar position on reducing SDS-PAGE. VH_A: VH of anti-CD3. VL_B: VL of anti-ALPPL2 (e.g., VL of SYLY). VH_B: VH of anti-ALPPL2 (e.g., VH of SYLY). VL_A: VL of anti-CD3.

FIG. 12. Binding of SYLY×CD3 DSDbody to human ALPP2 and ALPI measured by biolayer interferometry. Tips were loaded with either human ALPPL2-Fc or human ALPI-Fc, followed by an association step with 100 nM SYLY×CD3 DSDbody and then a dissociation step. Calculated affinites are shown in the graph. ND: not determined.

FIG. 13. Binding of SYLY×CD3 DSDbody to human and cynomolgus monkey CD3 epsilon measured by biolayer interferometry. Tips were loaded with either human or cynomolgus monkey CD3 epsilon, followed by an association step with 100 nM SYLY×CD3 DSDbody and then a dissociation step.

FIG. 14. Binding of the SYLY-based bispecific (DSDbody) to living M28 cells. M28 cells were incubated with Fabs for 1 h at RT and binding was analyzed by flow cytometry. Calculated affinity by curve fitting is 2.4 nM. There is no binding by the control bispecific YSC10×CD3 DSDbody that is built on a non-binding isotype control antibody YSC10.

FIG. 15. Binding of the FYIA-based bispecific (DSDbody) to living M28 cells. M28 cells were incubated with Fabs for 1 h at RT and binding was analyzed by flow cytometry. Calculated affinity by curve fitting is 14.8 nM. There is no binding by the control bispecific YSC10×CD3 DSDbody that is built on a non-binding isotype control antibody YSC10.

FIG. 16. Binding of the SYLY-based bispecific (DSDbody) to living SKOV3 cells. SKOV3 cells were incubated with Fabs for 1 h at RT and binding was analyzed by flow cytometry. Calculated affinity by curve fitting is 0.19 nM. There is no binding by the control bispecific YSC10×CD3 DSDbody that is built on a non-binding isotype control antibody YSC10.

FIG. 17. Binding of the FYIA-based bispecific (DSDbody) to living SKOV3 cells. SKOV3 cells were incubated with Fabs for 1 h at RT and binding was analyzed by flow cytometry. Calculated affinity by curve fitting is 14.2 nM. There is no binding by the control bispecific YSC10×CD3 DSDbody that is built on a non-binding isotype control antibody YSC10.

FIG. 18. The SYLY-based DSDbody BiTE was incubated with SKOV3 (target) cells in the presence of human PBMC (effector, E:T ratio 10:1) for 96h. Cell viability was assessed by Calcein AM. Calculated EC50 for the SYLY DSDbody is 0.3 pM. There is no killing by the control bispecific YSC10×CD3 DSDbody that is built on a non-binding isotype control antibody YSC10.

FIG. 19. The FYIA-based DSDbody BiTE was incubated with SKOV3 (target) cells in the presence of human PBMC (effector, E:T ratio 10:1) for 96h. Cell viability was assessed by Calcein AM. Calculated EC50 for the FYIA DSDbody is 62.8 pM. There is no killing by the control bispecific YSC10×CD3 DSDbody that is built on a non-binding isotype control antibody YSC10.

FIG. 20. The SYLY-based DSDbody BiTE was incubated with HEK293 cells stably expressing ALPP (target) in the presence of human PBMC (effector, E:T ratio 10:1) for 72h. Cell viability was assessed by Calcein AM. Calculated EC50 for the SYLY DSDbody is 8.5 pM, and >100 nM for the control bispecific YSC10×CD3 DSDbody.

FIG. 21. The SYLY-based DSDbody BiTE was incubated with HEK293 cells (target) in the presence of human PBMC (effector, E:T ratio 10:1) for 72h. Cell viability was assessed by Calcein AM. There is no apparent killing of HEK293 cells at antibody concentration up to 100 nM.

FIG. 22. Thermal-induced aggregation assay. The FYIA-based ALPPL2×CD3 DSDbody has an aggregation temperature (Tagg) of 65° C.

FIG. 23. Thermal-induced aggregation assay. The SYLY-based ALPPL2×CD3 DSDbody has an aggregation temperature (Tagg) of 70° C.

FIG. 24. Binding to AsPC1 cell by flow cytometry.

FIG. 25. In vitro cyto tox on AsPC1-luc.

### DEFINITIONS

As used in herein, the singular forms “a”, “an” and “the” include plural referents unless the content clearly dictates otherwise. Thus, for example, reference to “an antibody” optionally includes a combination of two or more such molecules, and the like.

As used herein, the term “antibody” means an isolated or recombinant binding agent that comprises the necessary variable region sequences to specifically bind an antigenic epitope. Therefore, an “antibody” as used herein is any form of antibody of any class or subclass or fragment thereof that exhibits the desired biological activity, e.g., binding a specific target antigen. Thus, it is used in the broadest sense and includes, but is not limited to, a monoclonal antibody (including full-length monoclonal antibodies), human antibodies, chimeric antibodies, single domain antibodies, such as nanobodies, diabodies, camelid-derived antibodies, monovalent antibodies, bivalent antibodies, multivalent antibodies, multispecific antibodies (e.g., bispecific antibodies), and antibody fragments including, but not limited to scFv, Fab, and the like so long as they exhibit the desired biological activity.

“Antibody fragments” comprise a portion of an intact antibody, for example, the antigen-binding or variable region of the intact antibody. Examples of antibody fragments include Fab, Fab′, F(ab′)₂, and Fv fragments; diabodies; linear antibodies; single-chain antibody molecules (e.g., scFv); and multispecific or multivalent antibodies formed from antibody fragments. A “Fab” fragment contains a variable and constant domain of the light chain and a variable domain and the first constant domain (CH1) of the heavy chain. A F(ab′)₂ fragment has a pair of Fab fragments that are generally covalently linked near their carboxy termini by hinge cysteines. Other chemical couplings of antibody fragments are also known. An “Fv” is a minimal antibody fragment that contains a complete antigen-recognition and binding site and is a dimer of one heavy- and one light-chain variable region domain.

The “class” of an antibody refers to the type of constant domain or constant region possessed by its heavy chain. There are five major classes of antibodies: IgA, IgD, IgE, IgG, and IgM, and may be further divided into subclasses (isotypes), e.g., IgG₁, IgG₂, IgG₃, IgG₄. The antibodies described herein can be of any of these classes or subclasses.

As used herein, “V-region” refers to an antibody variable region domain comprising the segments of Framework 1, CDR1, Framework 2, CDR2, and Framework 3, including CDR3 and Framework 4.

As used herein, “complementarity-determining region (CDR)” refers to the three hypervariable regions that interrupt the four “framework” regions of s variable domain. The CDRs are the primary contributors to binding to an epitope of an antigen. The CDRs of each heavy or light chain are referred to as CDR1, CDR2, and CDR3, numbered sequentially starting from the N-terminus.

The amino acid sequences of the CDRs and framework regions can be determined using various well-known definitions in the art, e.g., North, Kabat, Chothia, international ImMunoGeneTics database (IMGT), and AbM (see, e.g., North method. (see, e.g., North et al., J. Mol. Biol. 406(2):228-256, 2011; Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of CDRs are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs herein refer to CDRs determined according to the method of North (see, e.g., North et al., J. Mol. Biol. 406(2):228-256, 2011) unless indicated otherwise.

“Epitope” or “antigenic determinant” as used in the present disclosure in the context of antibody binding refers to a site on an antigen to which an antibody binds. Epitopes can be formed from contiguous amino acids and/or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). Binding of an antibody to an epitope can be influenced by other environmental factors, such as s the presence of calcium ions.

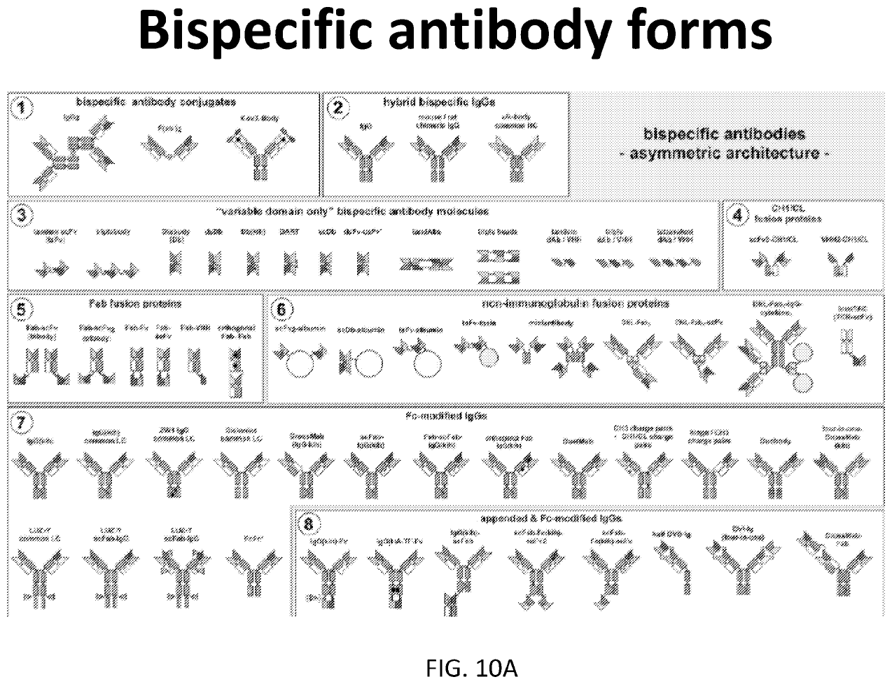

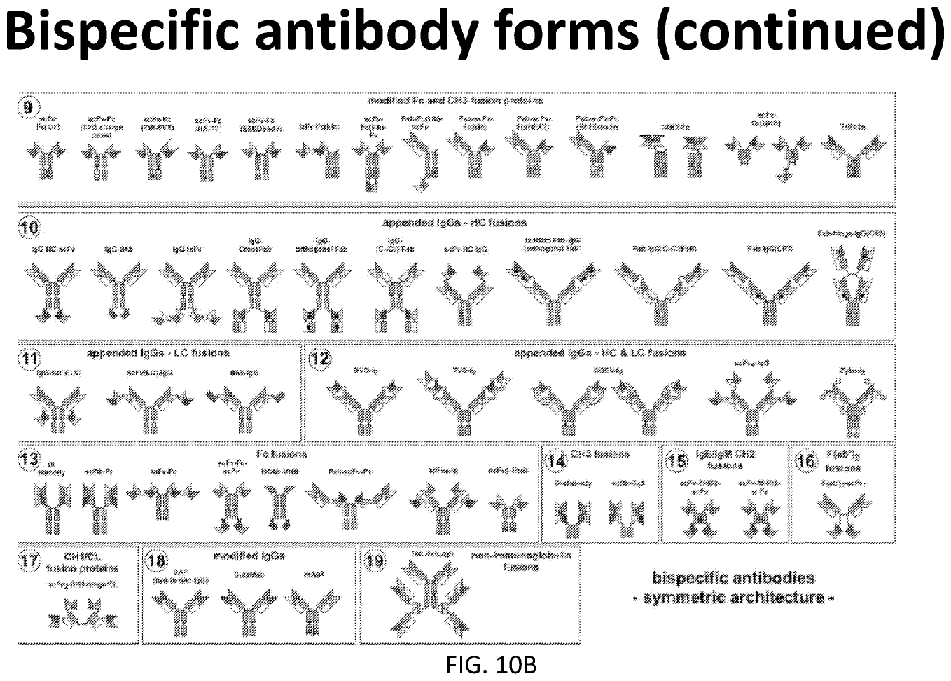

The term “bispecific antibody” as used herein, refers to an antibody that binds to two or more different epitopes. In some embodiments, a bispecific antibody binds to epitopes for two different target antigens. In some embodiments, a bispecific antibody binds to two different epitopes for the same target antigen. Bi-specific antibodies can be made in a number of ways. See, e.g., Brinkmann U, Kontermann R E. The making of bispecific antibodies. mAbs 2017; 9:182-212 and FIG. 10A-B. In some embodiments, the bi-specific antibodies described herein are diabodies or knob-in-a-hole IgG antibodies or otherwise use knob-in-a-hole technology. See, e.g., Xu, et al., MAbs 7(1):231-42 (2015).

The phrases “monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

As used herein, the term “specifically binds” to a target, e.g., human ALPP/ALPPL2, refers to a binding reaction whereby the antibody binds to the target with greater affinity, greater avidity, and/or greater duration than it binds to a different target. In some embodiments, a target-binding protein has at least 2-fold, 3-fold, 4-fold, 5-fold, 6-fold, 7-fold, 8-fold, 9-fold, 10-fold, 20-fold, 25-fold, 50-fold, 100-fold, 1,000-fold, 10,000-fold, or greater affinity for the target compared to an unrelated target when assayed under the same binding affinity assay conditions. The term “specific binding,” “specifically binds to,” or “is specific for” a particular target, as used herein, can be exhibited, for example, by a molecule (e.g., an antibody) having an equilibrium dissociation constant K_{D} for the target of, e.g., 10⁻² M or smaller, e.g., 10⁻³ M, 10⁻⁴ M, 10⁻⁵ M, 10⁻⁶ M, 10⁻⁷ M, 10⁻⁸ M, 10⁻⁹M, 10⁻¹⁰ M, 10⁻¹¹ M, or 10⁻¹² M. In some embodiments, an antibody has a K_{D} of less than 100 nM or less than 10 nM.

The term “treat” and “treatment” refer to both therapeutic treatment and prophylactic or preventive measures, wherein the object is to prevent or slow down an undesired physiological change or disorder. For purpose of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present invention, the pharmaceutically acceptable carrier must provide adequate pharmaceutical stability to the active ingredient. The nature of the carrier differs with the mode of administration. For example, for intravenous administration, an aqueous solution carrier is generally used; for oral administration, a solid carrier is preferred.

The phrase “inhibition of proliferation of a cell expressing ALPP and/or ALPPL2” as used herein, refers to the ability of an anti-ALPP/ALPPL2 antibody or immunoconjugate described herein to decrease, preferably to statistically significantly decrease proliferation of a cell expressing ALPP and/or ALPPL2 or a fragment thereof relative to the proliferation in the absence of the antibody or immunoconjugate. In one embodiment, the proliferation of a cell expressing ALPP/ALPPL2 or a fragment thereof (e-g, a cancer cell) may be decreased by at least 10%, or at least 20%, or at least 30%, or at least 40%, or at least 50%, or at least 60%, or at least 70%, or at least 80%, or at least 90%, or 100%) when the cells are contacted with the antibody or antigen binding portion thereof or an immunoconjugate described herein, relative to the proliferation measured in the absence of the antibody or antigen binding portion thereof or immunoconjugate (control). Cellular proliferation can be assayed using art recognized techniques which measure rate of cell division, the fraction of cells within a cell population undergoing cell division, and/or rate of cell loss from a cell population due to terminal differentiation or cell death (e.g., using a cell titer glow assay or thymidine incorporation).

An “isolated antibody,” as used herein, is intended to refer to an antibody that is substantially free of other antibodies having different antigenic specificities (e.g., an isolated antibody that specifically binds to ALPP and/or ALPL2 is substantially free of antibodies that specifically bind antigens other than ALPP and/or ALPPL2). In addition, an isolated antibody is typically substantially free of other cellular material and/or chemicals. In one embodiment, a combination of “isolated” monoclonal antibodies having different ALPP/ALPPL2 binding specificities are combined in a well-defined composition. Any of the antibodies provides herein can be provided as isolated antibodies.

The term “effective amount,” as used herein, refers to that amount of an anti-ALPP/ALPPL2 antibody or an antigen binding portion thereof and/or an immunoconjugate thereof, that is sufficient to effect treatment, prognosis or diagnosis of a disease associated with the growth and/or proliferation of ALPP/ALPPL2-positive cells (e.g., an ALPP/ALPPL2-positive cancer), as described herein, when administered to a subject. A therapeutically effective amount will vary depending upon the subject and disease condition being treated, the weight and age of the subject, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 g to about 1, 175 mg, about 500 g to about 1,150 mg, about 0.5 mg to about 1, 125 mg, about 1 mg to about 1, 100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an anti-ALPP/ALPPL2 antibody described herein and/or antigen binding portion thereof, and/or immunoconjugate thereof as described herein. Dosage regiments may be adjusted to provide the optimum therapeutic response. An effective amount is also one in which any toxic or detrimental effects (i.e., side effects) of an antibody or antigen binding portion thereof are minimized and/or outweighed by the beneficial effects.

An “effector” refers to any molecule or combination of molecules whose activity it is desired to deliver/into and/or localize at target cell. Effectors include, but are not limited to labels, cytotoxins, enzymes, growth factors, transcription factors, antibodies, drugs, etc.

The phrase “inhibiting the growth and/or proliferation”, e.g. of cancer cells includes inter alia inducing cellular apoptosis or other cell killing mechanisms, reducing the invasiveness of the cells, stalling the cells at a point in the cell cycle, and the like.

The term “immunoconjugate” refers to an antibody attached to one or more effectors or to a plurality of antibodies attached to one or more effectors. The term “immunoconjugate” is intended to include effectors chemically conjugated to the antibodies as well as antibodies expresses as a fusion protein where the antibody (or a portion thereof) is directly attached or attached through a linker to a peptide effector or to an effector comprising a peptide.

### DETAILED DESCRIPTION OF THE INVENTION

The inventors have developed improved anti-ALPPL2/ALPP antibodies that can be used as mono-specific or bi-specific antibodies. The antibodies can be used to kill tumor cells in a variety of ways. The antibodies are also useful for detection of tumor cells, and can be used for example as a companion diagnostic. Antibodies described herein bind to a cell that expresses or overexpresses ALPPL/ALPPL2. Exemplary non-limiting cells expressing ALPPL/ALPPL2 include but are not limited to, a mesothelioma cell, a testicular cancer cell, an endometrial cancer cell, a pancreatic cancer cell, an ovarian cancer cell, a non-small cell lung cancer cell, a gastric cancer cell, and a colon cancer cell.

As discussed in the Examples, previously described M25FYIA (aka FYIA) has been greatly improved to increase monovalent binding affinity to ALPPL2 while improving developability characteristics (e.g., avoiding charged or hydrophobic patches and removal of deamidation and isomerization motifs). In some embodiments, an antibody provided herein comprises a variable region that specifically binds to ALPPL2 and ALPP. The variable region can comprise, for example a heavy chain variable region and a light chain variable region. In some embodiments, complementarity determining region (CDR) 1, CDR2 and CDR3 of the heavy chain variable region are selected from the following sets: SEQ ID NOs: 20, 21, and 22; SEQ ID NOs: 23, 24, and 25; SEQ ID NOs: 26, 27, and 28; SEQ ID NOs: 29, 30, and 31; SEQ ID NOs: 32, 33, and 34; SEQ ID NOs: 35, 36, and 37; SEQ ID NOs: 38, 39, and 40; SEQ ID NOs: 41, 42, and 43; SEQ ID NOs: 44, 45, and 46; SEQ ID NOs: 47, 48, and 49; or SEQ ID NOs: 50, 51, and 52; and CDR1, CDR2, and CDR3 of the light chain variable region are selected from the following sets: SEQ ID NOs: 53, 54, and 55; SEQ ID NOs: 56, 57, and 58; SEQ ID NOs: 59, 60, and 61; SEQ ID NOs: 62, 63, and 64; SEQ ID NOs: 65, 66, and 67; SEQ ID NOs: 68, 69, and 70; SEQ ID NOs: 71, 72, and 73; or SEQ ID NOs: 74, 75, and 76.

In embodiments in which the heavy chain variable region CDRs comprise SEQ ID NOs: 20, 21, and 22 and the light chain variable region CDRs comprise SEQ ID NOs: 53, 54, and 55, the antibody will comprise framework sequences different than found in M25FYIA or other antibodies in PCT Publication No. WO2017/095823 having these CDR sequences. For example, the entire heavy chain variable region can comprise SEQ ID NO:1 and the light chain variable region can comprise SEQ ID NO:12.

In view of the similarity of the various heavy and light chain variable regions, it is believed any of the heavy chain variable regions (or CDRs thereof in a different framework) can be combined with any of the light chain variable regions (or CDRs thereof in a different framework) to form an antibody variable region that binds ALPPL2 and ALPP

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTFSSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 1 | FYIA | EVQLVESGGGLVQPGGSLRLSCAASGFTF | 20 | AASGFTESSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIAgerm_ | QSALTQPASVSGSPGQSIT | 68 | TGTSSDVGGYNYVS |
| | LC5 | ISCTGTSSDVGGYNYVSWY | 69 | YDVTDRPS |
| | | QQHPGKAPKLMIYDVTDRP | 70 | SSYTIASTLVV |
| | | SGVSNRFSGSKSGNTASLT | | |
| | | ISGLQAEDEADYYCSSYTI | | |
| | | ASTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIAgerm | QVQLVESGGGVVQPGGSLRL | 29 | AASGFTFSSYAMH |
| | 2 | SCAASGFTFSSYAMHWVRQA | 30 | VISYDGSNKY |
| | | PGKGLEWVAVISYDGSNKYY | 31 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 18 | FYIAgerm_ | QSALTQPASVSGSPGQSI | 71 | TGTSSDVGGYNYVS |
| | LC6 | TISCTGTSSDVGGYNYVS | 72 | YDVTDRPS |
| | | WYQQHPGKAPKLMIYDVT | 73 | SSYAIASTLVV |
| | | DRPSGVSNRFSGSKSGNT | | |
| | | ASLTISGLQAEDEADYYC | | |
| | | SSYAIASTLVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 4 | FYIAgerm | QVQLVESGGGVVQPGGSLRL | 29 | AASGFTFSSYAMH |
| | 2 | SCAASGFTFSSYAMHWVRQA | 30 | VISYDGSNKY |
| | | PGKGLEWVAVISYDGSNKYY | 31 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 19 | FYIAgerm_ | QSALTQPASVSGSPGQSI | 74 | TGTSSDVGGYNYVS |
| | LC7 | TISCTGTSSDVGGYNYVS | 75 | YDVTDRPS |
| | | WYQQHPGKAPKLMIYDVT | 76 | SSYAIASTPVV |
| | | DRPSGVSNRFSGSKSGNT | | |
| | | ASLTISGLQAEDEADYYC | | |
| | | SSYAIASTPVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_ | QVQLVESGGGVVQPGGSLRL | 32 | AASGFTFSSYDMH |
| | HC1 | SCAASGFTFSSYDMHWVRQA | 33 | VISYDGSNKY |
| | | PGKGLEWVAVISYDGSNKYY | 34 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 12 | FYIAgerm_ | QSALTQPASVSGSPGQSI | 53 | TGTSSDVGGYNYVS |
| | LC | TISCTGTSSDVGGYNYVS | 54 | YDVTNRPS |
| | | WYQQHPGKAPKLMIYDVT | 55 | SSYTIASTLVV |
| | | NRPSGVSNRFSGSKSGNT | | |
| | | ASLTISGLQAEDEADYYC | | |
| | | SSYTIASTLVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_ | QVQLVESGGGVVQPGGSLRL | 32 | AASGFTFSSYDMH |
| | HC1 | SCAASGFTFSSYDMHWVRQA | 33 | VISYDGSNKY |
| | | PGKGLEWVAVISYDGSNKYY | 34 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 13 | FYIAgerm_ | QSALTQPASVSGSPGQSI | 56 | TGTSSDVGGYNYVS |
| | SYLY_ | TISCTGTSSDVGGYNYVS | 57 | YDVTNYPS |
| | LC | WYQQHPGKAPKLMIYDVT | 58 | SSYTVATTLVV |
| | | NYPSGVSNRFSGSKSGNT | | |
| | | ASLTISGLQAEDEADYYC | | |
| | | SSYTVATTLVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_ | QVQLVESGGGVVQPGGSLRL | 32 | AASGFTFSSYDMH |
| | HC1 | SCAASGFTFSSYDMHWVRQA | 33 | VISYDGSNKY |
| | | PGKGLEWVAVISYDGSNKYY | 34 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 14 | FYIAgerm_ | QSALTQPASVSGSPGQSI | 59 | TGTSSDVGGYNYVS |
| | LC2 | TISCTGTSSDVGGYNYVS | 60 | YDVTNRPS |
| | | WYQQHPGKAPKLMIYDVT | 61 | SSYTIASTPVV |
| | | NRPSGVSNRFSGSKSGNT | | |
| | | ASLTISGLQAEDEADYYC | | |
| | | SSYTIASTPVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_ | QVQLVESGGGVVQPGGSLRL | 32 | AASGFTFSSYDMH |
| | HC1 | SCAASGFTFSSYDMHWVRQA | 33 | VISYDGSNKY |
| | | PGKGLEWVAVISYDGSNKYY | 34 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 15 | FYIAgerm_ | QSALTQPASVSGSPGQSI | 62 | TGTSSDVGGYNYVS |
| | LC3 | TISCTGTSSDVGGYNYVS | 63 | YDVTNRPS |
| | | WYQQHPGKAPKLMIYDVT | 64 | SSYTIASTFVV |
| | | NRPSGVSNRFSGSKSGNT | | |
| | | ASLTISGLQAEDEADYYC | | |
| | | SSYTIASTFVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_ | QVQLVESGGGVVQPGGSLRL | 32 | AASGFTFSSYDMH |
| | HC1 | SCAASGFTFSSYDMHWVRQA | 33 | VISYDGSNKY |
| | | PGKGLEWVAVISYDGSNKYY | 34 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 16 | FYIAgerm_ | QSALTQPASVSGSPGQSI | 65 | TGTSSDVGGYNYVS |
| | LC4 | TISCTGTSSDVGGYNYVS | 66 | YDVTNRPS |
| | | WYQQHPGKAPKLMIYDVT | 67 | SSYAIASTLVV |
| | | NRPSGVSNRFSGSKSGNT | | |
| | | ASLTISGLQAEDEADYYC | | |
| | | SSYAIASTLVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_HC1 | QVQLVESGGGVVQPGGSLRLSCAASGFTF | 32 | AASGEFTFSSYDMH |
| | | SSYDMHWVRQAPGKGLEWVAVISYDGSNK | 33 | VISYDGSNKY |
| | | YYADSVKGRFTISRDNSKNTLYLQMNSLR | 34 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIAgerm_LC5 | QSALTQPASVSGSPGQSITISCTGTS | 68 | TGTSSDVGGYNYVS |
| | | SDVGGYNYVSWYQQHPGKAPKLMIYD | 69 | YDVTDRPS |
| | | VTDRPSGVSNRFSGSKSGNTASLTIS | 70 | SSYTIASTLVV |
| | | GLQAEDEADYYCSSYTIASTLVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_HC1 | QVQLVESGGGVVQPGGSLRLSCAASGFTF | 32 | AASGFTFSSYDMH |
| | | SSYDMHWVRQAPGKGLEWVAVISYDGSNK | 33 | VISYDGSNKY |
| | | YYADSVKGRFTISRDNSKNTLYLQMNSLR | 34 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIAgerm_LC6 | QSALTQPASVSGSPGQSITISCTGTS | 71 | TGTSSDVGGYNYVS |
| | | SDVGGYNYVSWYQQHPGKAPKLMIYD | 72 | YDVTDRPS |
| | | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 5 | FYIAgerm2_HC1 | QVQLVESGGGVVQPGGSLRLSCAASGFTF | 32 | AASGFTFSSYDMH |
| | | SSYDMHWVRQAPGKGLEWVAVISYDGSNK | 33 | VISYDGSNKY |
| | | YYADSVKGRFTISRDNSKNTLYLQMNSLR | 34 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIAgerm_LC7 | QSALTQPASVSGSPGQSITISCTGTS | 74 | TGTSSDVGGYNYVS |
| | | SDVGGYNYVSWYQQHPGKAPKLMIYD | 75 | YDVTDRPS |
| | | VTDRPSGVSNRFSGSKSGNTASLTIS | 76 | SSYAIASTPVV |
| | | GLQAEDEADYYCSSYAIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIAgerm2_HC2 | QVQLVESGGGVVQPGGSLRLSCAASGFAF | 35 | AASGFAFSSYDMH |
| | | SSYDMHWVRQAPGKGLEWVAVISYDGSNK | 36 | VISYDGSNKY |
| | | YYADSVKGRFTISRDNSKNTLYLQMNSLR | 37 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIAgerm_LC | QSALTQPASVSGSPGQSITISCTGTS | 53 | TGTSSDVGGYNYVS |
| | | SDVGGYNYVSWYQQHPGKAPKLMIYD | 54 | YDVTNRPS |
| | | VTNRPSGVSNRFSGSKSGNTASLTIS | 55 | SSYTIASTLVV |
| | | GLQAEDEADYYCSSYTIASTLVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIAgerm2_HC2 | QVQLVESGGGVVQPGGSLRLSCAASGFAF | 35 | AASGFAFSSYDMH |
| | | SSYDMHWVRQAPGKGLEWVAVISYDGSNK | 36 | VISYDGSNKY |
| | | YYADSVKGRFTISRDNSKNTLYLQMNSLR | 37 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIAgerm_SYLY_LC | QSALTQPASVSGSPGQSITISCTGTS | 56 | TGTSSDVGGYNYVS |
| | | SDVGGYNYVSWYQQHPGKAPKLMIYD | 57 | YDVTNYPS |
| | | VTNYPSGVSNRFSGSKSGNTASLTIS | 58 | SSYTVATTLVV |
| | | GLQAEDEADYYCSSYTVATTLVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

In combination with the light chain variable region comprising the CDRs of, or the entire light chain variable sequence, displayed below:

| | | | | |
|---|---|---|---|---|
| 6 | FYIAgerm2_HC2 | QVQLVESGGGVVQPGGSLRLSCAASGFAF | 35 | AASGFAFSSYDMH |
| | | SSYDMHWVRQAPGKGLEWVAVISYDGSNK | 36 | VISYDGSNKY |
| | | YYADSVKGRFTISRDNSKNTLYLQMNSLR | 37 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIAgerm_LC2 | QSALTQPASVSGSPGQSITISCTGTS | 59 | TGTSSDVGGYNYVS |
| | | SDVGGYNYVSWYQQHPGKAPKLMIYD | 60 | YDVTNRPS |
| | | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIAgerm2_HC2 | QVQLVESGGGVVQPGGSLRLSCAASGFAF | 35 | AASGFAFSSYDMH |
| | | SSYDMHWVRQAPGKGLEWVAVISYDGSNK | 36 | VISYDGSNKY |
| | | YYADSVKGRFTISRDNSKNTLYLQMNSLR | 37 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIAgerm_LC3 | QSALTQPASVSGSPGQSITISCTGTS | 62 | TGTSSDVGGYNYVS |
| | | SDVGGYNYVSWYQQHPGKAPKLMIYD | 63 | YDVTNRPS |
| | | VTNRPSGVSNRFSGSKSGNTASLTIS | 64 | SSYTIASTFVV |
| | | GLQAEDEADYYCSSYTIASTFVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIAgerm2_HC2 | QVQLVESGGGVVQPGGSLRLSCAASGFAF | 35 | AASGFAFSSYDMH |
| | | SSYDMHWVRQAPGKGLEWVAVISYDGSNK | 36 | VISYDGSNKY |
| | | YYADSVKGRFTISRDNSKNTLYLQMNSLR | 37 | AKEGDSSRWSYDL |
| | | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIA | QVQLVESGGGVVQPGGSLRLSCAASGFAF | 35 | AASGFAFSSYDMH |
| | germ | SSYDMHWVRQAPGKGLEWVAVISYDGSNK | 36 | VISYDGSNKY |
| | 2_HC | YYADSVKGRFTISRDNSKNTLYLQMNSLR | 37 | AKEGDSSRWSYDL |
| | 2 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIA | QVQLVESGGGVVQPGGSLRLSCAASGFAF | 35 | AASGFAFSSYDMH |
| | germ | SSYDMHWVRQAPGKGLEWVAVISYDGSNK | 36 | VISYDGSNKY |
| | 2_HC | YYADSVKGRFTISRDNSKNTLYLQMNSLR | 37 | AKEGDSSRWSYDL |
| | 2 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 6 | FYIA | QVQLVESGGGVVQPGGSLRLSCAASGFAF | 35 | AASGFAFSSYDMH |
| | germ | SSYDMHWVRQAPGKGLEWVAVISYDGSNK | 36 | VISYDGSNKY |
| | 2_HC | YYADSVKGRFTISRDNSKNTLYLQMNSLR | 37 | AKEGDSSRWSYDL |
| | 2 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIA | QVQLVESGGGVVQPGGSLRLSCAASGFTF | 38 | AASGFTFSSYAMH |
| | germ | SSYAMHWVRQAPGKGLEWVAVISYDGSNK | 39 | VISYDGSNKY |
| | 2_HC | YYADSVKGRFTISRDNSKNTLYLQMNSLR | 40 | AKEGDSSRWGYDL |
| | 3 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIA | QVQLVESGGGVVQPGGSLRLSCAASGFTF | 38 | AASGFTFSSYAMH |
| | germ | SSYAMHWVRQAPGKGLEWVAVISYDGSNK | 39 | VISYDGSNKY |
| | 2_HC | YYADSVKGRFTISRDNSKNTLYLQMNSLR | 40 | AKEGDSSRWGYDL |
| | 3 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIA | QVQLVESGGGVVQPGGSLRLSCAASGFTF | 38 | AASGFTFSSYAMH |
| | germ | SSYAMHWVRQAPGKGLEWVAVISYDGSNK | 39 | VISYDGSNKY |
| | 2_HC | YYADSVKGRFTISRDNSKNTLYLQMNSLR | 40 | AKEGDSSRWGYDL |
| | 3 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIA | QVQLVESGGGVVQPGGSLRLSCAASGFTF | 38 | AASGFTFSSYAMH |
| | germ | SSYAMHWVRQAPGKGLEWVAVISYDGSNK | 39 | VISYDGSNKY |
| | 2_HC | YYADSVKGRFTISRDNSKNTLYLQMNSLR | 40 | AKEGDSSRWGYDL |
| | 3 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIA | QVQLVESGGGVVQPGGSLRLSCAA | 38 | AASGFTFSSYAMH |
| | germ2_ | SGFTFSSYAMHWVRQAPGKGLEWV | 39 | VISYDGSNKY |
| | HC3 | AVISYDGSNKYYADSVKGRFTISR | 40 | AKEGDSSRWGYDL |
| | | DNSKNTAEDTAVYYCAKEGDSSRW | | |
| | | GYDLWGRGTLVTVSS | | |

In combination with the light chain variable region comprising the CDRs of, or the entire light chain variable sequence, displayed below:

| | | | | |
|---|---|---|---|---|
| 16 | FYIAgerm_ | QSALTQPASVSGSPGQSITI | 65 | TGTSSDVGGYNYVS |
| | LC4 | SCTGTSSDVGGYNYVSWYQQ | 66 | YDVTNRPS |
| | | HPGKAPKLMIYDVTNRPSGV | 67 | SSYAIASTLVV |
| | | SNRFSGSKSGNTASLTISGL | | |
| | | QAEDEADYYCSSYAIASTLV | | |
| | | VFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIAgerm2_ | QVQLVESGGGVVQPGGSLRLS | 38 | AASGFTESSYAMH |
| | HC3 | CAASGFTFSSYAMHWVRQAPG | 39 | VISYDGSNKY |
| | | KGLEWVAVISYDGSNKYYADS | 40 | AKEGDSSRWGYDL |
| | | VKGRFTISRDNSKNTLYLQMN | | |
| | | SLRAEDTAVYYCAKEGDSSRW | | |
| | | GYDLWGRGTLVTVSS | | |

In combination with the light chain variable region comprising the CDRs of, or the entire light chain variable sequence, displayed below:

| | | | | |
|---|---|---|---|---|
| 17 | FYIAger | QSALTQPASVSGSPGQSITIS | 68 | TGTSSDVGGYNYVS |
| | LC5 | CTGTSSDVGGYNYVSWYQQHP | 69 | YDVTDRPS |
| | | GKAPKLMIYDVTDRPSGVSNR | 70 | SSYTIASTLVV |
| | | FSGSKSGNTASLTISGLQAED | | |
| | | EADYYCSSYTIASTLVVFGGG | | |
| | | TKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIAgerm2_ | QVQLVESGGGVVQPGGSLRL | 38 | AASGFTFSSYAMH |
| | HC3 | SCAASGFTFSSYAMHWVRQA | 39 | VISYDGSNKY |
| | | PGKGLEWVAVISYDGSNKYY | 40 | AKEGDSSRWGYDL |
| | | ADSVKGRFTISRDNSKNTLY | | |
| | | LQMNSLRAEDTAVYYCAKEG | | |
| | | DSSRWGYDLWGRGTLVTVSS | | |

In combination with the light chain variable region comprising the CDRs of, or the entire light chain variable sequence, displayed below:

| | | | | |
|---|---|---|---|---|
| 18 | FYIAgerm_ | QSALTQPASVSGSPGQSIT | 71 | TGTSSDVGGYNYVS |
| | LC6 | ISCTGTSSDVGGYNYVSWY | 72 | YDVTDRPS |
| | | QQHPGKAPKLMIYDVTDRP | 73 | SSYAIASTLVV |
| | | SGVSNRFSGSKSGNTASLT | | |
| | | ISGLQAEDEADYYCSSYAI | | |
| | | ASTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 7 | FYIAgerm2_ | QVQLVESGGGVVQPGGSLRL | 38 | AASGFTFSSYAMH |
| | HC3 | SCAASGFTFSSYAMHWVRQA | 39 | VISYDGSNKY |
| | | PGKGLEWVAVISYDGSNKYY | 40 | AKEGDSSRWGYDL |
| | | ADSVKGRFTISRDNSKNTLY | | |
| | | LQMNSLRAEDTAVYYCAKEG | | |
| | | DSSRWGYDLWGRGTLVTVSS | | |

In combination with the light chain variable region comprising the CDRs of, or the entire light chain variable sequence, displayed below:

| | | | | |
|---|---|---|---|---|
| 19 | FYIAgerm_ | QSALTQPASVSGSPGQSI | 74 | TGTSSDVGGYNYVS |
| | LC7 | TISCTGTSSDVGGYNYVS | 75 | YDVTDRPS |
| | | WYQQHPGKAPKLMIYDVT | 76 | SSYAIASTPVV |
| | | DRPSGVSNRFSGSKSGNT | | |
| | | ASLTISGLQAEDEADYYC | | |
| | | SSYAIASTPVVFGGGTKL | | |
| | | TVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIAgerm_ | QVQLVESGGGVVQPGGSLRLS | 41 | AASGFTFSSYAMH |
| | HC4 | CAASGFTFSSYAMHWVRQAPG | 42 | VVSYDGSNKY |
| | | KGLEWVAVVSYDGSNKYYADS | 43 | AKEGDSSRWSYDL |
| | | VKGRFTISRDNSKNTLYLQMN | | |
| | | SLRAEDTAVYYCAKEGDSSRW | | |
| | | SYDLWGRGTLVTVSS | | |

In combination with the light chain variable region comprising the CDRs of, or the entire light chain variable sequence, displayed below:

| | | | | |
|---|---|---|---|---|
| 12 | FYIAgerm_ | QSALTQPASVSGSPGQSIT | 53 | TGTSSDVGGYNYVS |
| | LC | ISCTGTSSDVGGYNYVSWY | 54 | YDVTNRPS |
| | | QQHPGKAPKLMIYDVTNRP | 55 | SSYTIASTLVV |
| | | SGVSNRFSGSKSGNTASLT | | |
| | | ISGLQAEDEADYYCSSYTI | | |
| | | ASTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIAgerm2_ | QVQLVESGGGVVQPGGSLRL | 41 | AASGFTFSSYAMH |
| | HC4 | SCAASGFTFSSYAMHWVRQA | 42 | VVSYDGSNKY |
| | | PGKGLEWVAVVSYDGSNKYY | 43 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 13 | FYIAgerm_ | QSALTQPASVSGSPGQSIT | 56 | TGTSSDVGGYNYVS |
| | SYLY_LC | ISCTGTSSDVGGYNYVSWY | 57 | YDVTNYPS |
| | | QQHPGKAPKLMIYDVTNYP | 58 | SSYTVATTLVV |
| | | SGVSNRFSGSKSGNTASLT | | |
| | | ISGLQAEDEADYYCSSYTV | | |
| | | ATTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIAgerm2_ | QVQLVESGGGVVQPGGSLRL | 41 | AASGFTFSSYAMH |
| | HC4 | SCAASGFTFSSYAMHWVRQA | 42 | VVSYDGSNKY |
| | | PGKGLEWVAVVSYDGSNKYY | 43 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 14 | FYIAgerm_ | QSLATQPASVSGSPGQSIT | 59 | TGTSSDVGGYNYVS |
| | LC2 | ISCTGTSSDVGGYNYVSWY | 60 | YDVTNRPS |
| | | QQHPGKAPKLMIYDVTNRP | 61 | SSYTIASTPVV |
| | | SGVSNRFSGSKSGNTASLT | | |
| | | ISGLQAEDEADYYCSSYTI | | |
| | | ASTPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence dis played below:

| | | | | |
|---|---|---|---|---|
| 8 | FYIAgerm2_ | QVQLVESGGGVVQPGGSLRL | 41 | AASGFTFSSYAMH |
| | HC4 | SCAASGFTFSSYAMHWVRQA | 42 | VVSYDGSNKY |
| | | PGKGLEWVAVVSYDGSNKYY | 43 | AKEGDSSRWSYDL |
| | | ADSVKGRFTISRDNSKNTLY | | |
| | | LQMNSLRAEDTAVYYCAKEG | | |
| | | DSSRWSYDLWGRGTLVTVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIA | QVQLVESGGGVVQPGGSLRLSCAASGFTF | 41 | AASGFTESSYAMH |
| | germ | SSYAMHWVRQAPGKGLEWVAVVSYDGSNK | 42 | VVSYDGSNKY |
| | 2_HC | YYADSVKGRFTISRDNSKNTLYLQMNSLR | 43 | AKEGDSSRWSYDL |
| | 4 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIA | QVQLVESGGGVVQPGGSLRLSCAASGFTF | 41 | AASGFTESSYAMH |
| | germ | SSYAMHWVRQAPGKGLEWVAVVSYDGSNK | 42 | VVSYDGSNKY |
| | 2_HC | YYADSVKGRFTISRDNSKNTLYLQMNSLR | 43 | AKEGDSSRWSYDL |
| | 4 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIA | QVQLVESGGGVVQPGGSLRLSCAASGFTF | 41 | AASGFTESSYAMH |
| | germ | SSYAMHWVRQAPGKGLEWVAVVSYDGSNK | 42 | VVSYDGSNKY |
| | 2_HC | YYADSVKGRFTISRDNSKNTLYLQMNSLR | 43 | AKEGDSSRWSYDL |
| | 4 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 8 | FYIA | QVQLVESGGGVVQPGGSLRLSCAASGFTF | 41 | AASGFTESSYAMH |
| | germ | SSYAMHWVRQAPGKGLEWVAVVSYDGSNK | 42 | VVSYDGSNKY |
| | 2_HC | YYADSVKGRFTISRDNSKNTLYLQMNSLR | 43 | AKEGDSSRWSYDL |
| | 4 | AEDTAVYYCAKEGDSSRWSYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_HC | YYADSVKGRFTISRDNSKNTLYLQMNSLR | 46 | AKEGDSSRWGYDL |
| | 6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_HC | YYADSVKGRFTISRDNSKNTLYLQMNSLR | 46 | AKEGDSSRWGYDL |
| | 6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_HC | YYADSVKGRFTISRDNSKNTLYLQMNSLR | 46 | AKEGDSSRWGYDL |
| | 6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_ | YYADSVKGRFTISRDNSKNTLYLQMNSLR | 46 | AKEGDSSRWGYDL |
| | HC6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ | SDVGGYNYVSWYQQHPGKAPKLMIYD | 63 | YDVTNRPS |
| | _LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_ | YYADSVKGRFTISRDNSKNTLYLQMNSLR | 46 | AKEGDSSRWGYDL |
| | HC6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTESSYAMH |
| | germ | SSYAMHWVRQAPGKGLEWVAVVSYDGSNK | 45 | VVSYDGSNKY |
| | 2_ | YYADSVKGRFTISRDNSKNTLYLQMNSLR | 46 | AKEGDSSRWGYDL |
| | HC6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCEGGGTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTESSYAMH |
| | germ | SSYAMHWVRQAPGKGLEWVAVVSYDGSNK | 45 | VVSYDGSNKY |
| | 2_ | YYADSVKGRFTISRDNSKNTLYLQMNSLR | 46 | AKEGDSSRWGYDL |
| | HC6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 9 | FYIA | QVQLVESGGGVVQPGGSLRLSCAASGFTF | 44 | AASGFTESSYAMH |
| | germ | SSYAMHWVRQAPGKGLEWVAVVSYDGSNK | 45 | VVSYDGSNKY |
| | 2_ | YYADSVKGRFTISRDNSKNTLYLQMNSLR | 46 | AKEGDSSRWGYDL |
| | HC6 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTGTS | 74 | TGTSSDVGGYNYVS |
| | germ | SDVGGYNYVSWYQQHPGKAPKLMIYD | 75 | YDVTDRPS |
| | _LC7 | VTDRPSGVSNRFSGSKSGNTASLTIS | 76 | SSYAIASTPVV |
| | | GLQAEDEADYYCSSYAIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_ | YYADSVKGRFTISRDNSKNTLYLQMNSLR | 49 | AKEGDSSRWGYDL |
| | HC7 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_ | YYADSVKGRFTISRDNSKNTLYLQMNSLR | 49 | AKEGDSSRWGYDL |
| | HC7 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 13 | FYIA | QSALTQPASVSGSPGQSITISCTGTS | 56 | TGTSSDVGGYNYVS |
| | germ_ | SDVGGYNYVSWYQQHPGKAPKLMIYD | 57 | YDVTNYPS |
| | SYLY_ | VTNYPSGVSNRFSGSKSGNTASLTIS | 58 | SSYTVATTLVV |
| | LC | GLQAEDEADYYCSSYTVATTLVVFGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_ | YYADSVKGRFTISRDNSKNTLYLQMNSLR | 49 | AKEGDSSRWGYDL |
| | HC7 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTGTS | 74 | TGTSSDVGGYNYVS |
| | germ_ | SDVGGYNYVSWYQQHPGKAPKLMIYD | 75 | YDVTDRPS |
| | LC7 | VTDRPSGVSNRFSGSKSGNTASLTIS | 76 | SSYAIASTPVV |
| | | GLQAEDEADYYCSSYAIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTESSYDMH |
| | germ | SSYDMHWVRQAPGKGLEWVAVVSYDGSNK | 51 | VVSYDGSNKY |
| | 2_ | YYADSVKGRFTISRDNSKNTLYLQMNSLR | 52 | AKEGDSSRWGYDL |
| | HC8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTESSYDMH |
| | germ | SSYDMHWVRQAPGKGLEWVAVVSYDGSNK | 51 | VVSYDGSNKY |
| | 2_ | YYADSVKGRFTISRDNSKNTLYLQMNSLR | 52 | AKEGDSSRWGYDL |
| | HC8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 11 | FYIA | QVQLVESGGGVVQPGGSLRLSCAASGFTF | 50 | AASGFTESSYDMH |
| | germ | SSYDMHWVRQAPGKGLEWVAVVSYDGSNK | 51 | VVSYDGSNKY |
| | 2_ | YYADSVKGRFTISRDNSKNTLYLQMNSLR | 52 | AKEGDSSRWGYDL |
| | HC8 | AEDTAVYYCAKEGDSSRWGYDLWGRGTLV | | |
| | | TVSS | | |

In some embodiments, an antibody described herein comprises a variable region that specifically binds to ALPPL2 and ALPP, wherein the heavy chain variable region comprising the CDRs of, or the entire heavy chain variable sequence, displayed below:

In combination with the light chain variable region comprising the CDRs of, or the entire light chain variable sequence, displayed below:

| | | | | |
|---|---|---|---|---|
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_HC | YYADSVKGRFTISRDNSKNTLYLQMNSLR | 52 | AKEGDSSRWGYDL |
| | 8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below

| | | | | |
|---|---|---|---|---|
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_HC | YYADSVKGRFTISRDNSKNTLYLQMNSLR | 52 | AKEGDSSRWGYDL |
| | 8 | AEDTAVYYCAKEGDSSRWGYDLWGRGTLV | | |

In combination with the light chain variable region comprising the CDRs of, or the entire light chain variable sequence, displayed below:

| | | | | |
|---|---|---|---|---|
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_HC | YYADSVKGRFTISRDNSKNTLYLQMNSLR | 52 | AKEGDSSRWGYDL |
| | 8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | | VTDRPSGVSNRFSGSKSGNTASLTIS | 70 | SSYTIASTLVV |
| | LC5 | GLQAEDEADYYCSSYTIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_HC | YYADSVKGRFTISRDNSKNTLYLQMNSLR | 52 | AKEGDSSRWGYDL |
| | 8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |

In some embodiments, an antibody described herein comprises a variable region that specifically binds to ALPPL2 and ALPP, wherein the heavy chain variable region comprising the CDRs of, or the entire heavy chain variable sequence, displayed below:

| | | | | |
|---|---|---|---|---|
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_ | YYADSVKGRFTISRDNSKNTLYLQMNSLR | 52 | AKEGDSSRWGYDL |
| | HC8 | AEDTAVYYCAKEGDSSRWGYDLWGRGTLV | | |
| | | TVSS | | |

In combination with the light chain variable region comprising the CDRs of, or the entire light chain variable sequence, displayed below:

| | | | | |
|---|---|---|---|---|
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

In some embodiments, the antibody comprises a heavy chain variable region having at least 90% identity (e.g., 90%, 91%, 92%, 93%, 94%, 95%, 96%, 97%, 98%, 99%, or 100%) to the amino acid sequence of any one of SEQ ID NOS:1-11. In some embodiments, the antibody comprises a light chain variable region having at least 90% identity (e.g., 90%, 91%, 92%, 93%, 94%, 95%, 96%, 97%, 98%, 99%, or 100%) to the amino acid sequence of any one of SEQ ID NOS: 12-19.

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a Lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which are incorporated herein by reference.

In certain embodiments, specific mutations of antibodies can be made to alter the glycosylation of the polypeptide. Such mutations may be selected to introduce or eliminate one or more glycosylation sites, including but not limited to, O-linked or N-linked glycosylation sites. In certain embodiments, the proteins have glycosylation sites and patterns unaltered relative to the naturally-occurring proteins. In certain embodiments, a variant of proteins includes a glycosylation variant wherein the number and/or type of glycosylation sites have been altered relative to the naturally-occurring proteins. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue. The substitution of amino acid residues to create this sequence provides a potential new site for the addition of an N-linked carbohydrate chain. Alternatively, substitutions which eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created.

In some embodiments, the antibody is tetrameric or a fragment thereof. In some embodiments, the antibodies are single chain antibodies, the VH and VL domains comprising the antibody can be joined directly together or by a peptide linker. Illustrative peptide linkers include, but are not limited to GGGGS GGGGS GGGGS (SEQ ID NO:89), GGGGS GGGGS (SEQ ID NO: 90), GGGGS (SEQ ID NO: 91), GS GGGGS GGGGS GGS GGGGS (SEQ ID NO: 92), S GGGGS (SEQ ID NO: 93), GGGS (SEQ ID NO:94), VPGV (SEQ ID NO: 95), VPGVG (SEQ ID NO: 96), GVPGVG (SEQ ID NO: 97), GVGVPGVG (SEQ ID NO: 98), VPGVGVPGVG (SEQ ID NO: 99), GGSSRSS (SEQ ID NO: 100), and GGSSRSSSSGGGGSGGGG (SEQ ID NO: 101), and the like.

In various embodiments antibodies described herein can be produced by chemical synthesis or can be recombinantly expressed. For example, using the sequence information provided herein, the anti-ALPPL2 specific antibodies described herein or variants thereof, can be chemically synthesized using well known methods of peptide synthesis. Solid phase synthesis in which the C-terminal amino acid of the sequence is attached to an insoluble support followed by sequential addition of the remaining amino acids in the sequence is one preferred method for the chemical synthesis of single chain antibodies. Techniques for solid phase synthesis are described by Barany and Merrifield, Solid Phase Peptide Synthesis; pp. 3-284 in The Peptides: Analysis, Synthesis, Biology. Vol. 2: Special Methods in Peptide Synthesis, Part A., Merrifield et al. (1963) J. Am. Chem. Soc, 85: 2149-2156, and Stewart et al. (1984) Solid Phase Peptide Synthesis, 2nd ed. Pierce Chem. Co., Rockford, 111.

In certain embodiments, the anti-ALPPL2/ALPPL specific antibodies described herein or variants thereof, are recombinantly expressed using methods well known to those of skill in the art. For example, using the sequence information provided herein, nucleic acids encoding the desired antibody can be prepared according to a number of standard methods known to those of skill in the art. The nucleic acids are transfected into host cells that then express the desired antibody or a chain thereof.

Molecular cloning techniques to achieve these ends are known in the art. A wide variety of cloning and in vitro amplification methods are suitable for the construction of recombinant nucleic acids. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, CA (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Natl Acad. Sci. USA 86: 10029-10033. In addition, detailed protocols for the expression of antibodies are also provided by Liu et al. (2004) Cancer Res. 64: 704-710, Poul et al. (2000) J. Mol. Biol. 301: 1149-1161, and the like.

Using the known and/or identified sequences (e.g. VH and/or VL sequences) of the antibodies provided herein other antibody forms can readily be created. Such forms include, but are not limited to multivalent antibodies, full antibodies, scFv, (scFv′)2, Fab, (Fab′)2, chimeric antibodies, and the like. For example, to create (scFv′)2 antibodies, two anti-ALPP/ALPPL2 antibodies are joined, either through a linker {e.g., a carbon linker, a peptide, etc.) or through a disulfide bond between, for example, two cysteins. Thus, for example, to create disulfide linked scFv, a cysteine residue can be introduced by site directed mutagenesis at the carboxy-terminus of the antibodies described herein. An scFv can be expressed from this construct, purified by EVIAC, and analyzed by gel filtration. To produce (scFv′)2 dimers, the cysteine is reduced by incubation with 1 mM 3-mercaptoethanol, and half of the scFv blocked by the addition of DT B. Blocked and unblocked scFvs are incubated together to form (scFv′)2 and the resulting material can be analyzed by gel filtration. The affinity of the resulting dimmer can be determined using standard methods, e.g. by BIAcore. In one illustrative embodiment, the (scFv′)2 dimer is created by joining the scFv′ fragments through a linker, e.g., through a peptide linker. This can be accomplished by a wide variety of means well known to those of skill in the art. For example, one approach is described by Holliger et al. (1993) Proc. Natl. Acad. Sci. USA, 90: 6444-6448 (see also WO 94/13804).

Using the VH and/or VL sequences provided herein, Fabs and (Fab′)2 dimers can also readily be prepared. Fab is a light chain joined to VH-CH1 by a disulfide bond and can readily be created using standard methods known to those of skill in the art. The F(ab)′2 can be produced by dimerizing the Fab, e.g. as described above for the (scFv′)2 dimer.

The antibodies contemplated herein also include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; Morrison et al. (1984) Proc. Natl. Acad. Sci. 81: 6851-6855, etc.). Chimeric antibodies are antibodies comprising portions from two different species (e.g. a human and non-human portion). Typically, the antigen combining region (or variable region) of a chimeric antibody is derived from a one species source and the constant region of the chimeric antibody (which confers biological effector function to the immunoglobulin) is derived from another source. A large number of methods of generating chimeric antibodies are well known to those of skill in the art {see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369, and PCT Application WO 91/0996).

In another embodiment, this invention provides for intact, fully human anti-ALPP/ALPPL2 antibodies. Such antibodies can readily be produced in a manner analogous to making chimeric human antibodies. In this instance, the VH and VL domains described herein are fully human and can readily be engineered into a substantially complete antibody (e.g., IgG, IgA, IgM, etc.).

In certain embodiments using the sequence information provided herein, the anti-ALPP/ALPPL2 antibodies can be constructed as unibodies. UniBody are antibody technology that produces a stable, smaller antibody format with an anticipated longer therapeutic window than certain small antibody formats. In certain embodiments unibodies are produced from IgG4 antibodies by eliminating the hinge region of the antibody. Unlike the full size IgG4 antibody, the half molecule fragment is very stable and is termed a uniBody. Halving the IgG4 molecule leaves only one area on the UniBody that can bind to a target. Methods of producing unibodies are described in detail in PCT Publication WO2007/059782, which is incorporated herein by reference in its entirety {see, also, Kolfschoten et al. (2007) Science 31′: 1554-1557).

In certain embodiments the sequence information provided herein is used to construct affibody molecules that bind ALPP/ALPPL2. Affibody molecules are class of affinity proteins based on a 58-amino acid residue protein domain, derived from one of the IgG-binding domains of staphylococcal protein A. This three helix bundle domain has been used as a scaffold for the construction of combinatorial phagemid libraries, from which affibody variants that target the desired molecules can be selected using phage display technology {see, e.g., Nord et al. (1997) Nat. Biotechnol. 15: 772-777; Ronmark et al. (2002) Eur. J. Biochem., 269: 2647-2655.). Details of Affibodies and methods of production are known to those of skill {see, e.g., U.S. Pat. No. 5,831,012 which is incorporated herein by reference in its entirety).

It will be recognized that the antibodies described above can be provided as whole intact antibodies {e.g., IgG), antibody fragments, or single chain antibodies, using methods well known to those of skill in the art. In addition, while the antibody can be from essentially any mammalian species, to reduce immunogenicity, it is desirable to use an antibody that is of the species in which the antibody and/or immunoconjugate is to be used. In other words, for use in a human, it is desirable to use a human, humanized, or chimeric human antibody.

Any of the antibodies described herein, and specifically in some embodiments, monospecific antibodies described herein, can be linked to an effector molecule. Anti-ALPP/ALPPL2 immunoconjugates can be formed by conjugating the antibodies or antigen binding portions thereof described herein to an effector (e.g., a detectable label, another therapeutic agent, etc.). Illustrative therapeutic agents include, but are not limited to, for example, a cytotoxic or cytostatic agent (e.g., a chemotherapeutic agent), a toxin (e.g. an enzymatically active toxin of bacterial, fungal, plant or animal origin, or fragments thereof), a radioactive isotope (e.g., a radioconjugate), or a second antibody.

In certain embodiments, the anti-ALPP/ALPPL2 immunoconjugates can be used to direct detectable labels to a tumor site or otherwise detect the presence of the cancer cell. This can facilitate tumor detection and/or localization. It can be effective for detecting primary tumors, or, in certain embodiments, secondary tumors produced by cancers that express ALPPL2 (e.g., cancers including, but not limited to mesothelioma, testicular cancer, endometrial cancer, ovarian cancer, pancreatic cancer, and non-small cell lung cancer).

Thus, in certain embodiments, the effector comprises a detectable label. Suitable detectable labels include, but are not limited to radio-opaque labels, nanoparticles, PET labels, MRI labels, radioactive labels, and the like. Among the radionuclides and useful in various embodiments, gamma-emitters, positron-emitters, x-ray emitters and fluorescence-emitters are suitable for localization, diagnosis and/or staging, and/or therapy, while beta and alpha-emitters and electron and neutron-capturing agents, such as boron and uranium, also can be used for therapy.

In various embodiments the detectable labels can be used in conjunction with an external detector and/or an internal detector and provide a means of effectively localizing and/or visualizing cancer cells expressing ALPPL2. Such detection/visualization can be useful in various contexts including, but not limited to pre-operative and intraoperative settings. Thus, in certain embodiment this invention relates to a method of intraoperatively detecting cancers that express ALPPL2 in the body of a mammal. These methods typically involve administering to the mammal a composition comprising, in a quantity sufficient for detection by a detector (e.g. a gamma detecting probe), an anti-ALPPL2 antibody labeled with a detectable label as described herein, and, after allowing the active substance to be taken up by the target tissue, and preferably after blood clearance of the label, subjecting the mammal to a radioimmunodetection technique in the relevant area of the body, e.g. by using a gamma detecting probe.

In certain embodiments the label-bound antibody can be used in the technique of radioguided surgery, wherein relevant tissues in the body of a subject can be detected and located intraoperatively by means of a detector, e.g. a gamma detecting probe.

The surgeon can, intraoperatively, use this probe to find the tissues in which uptake of the compound labeled with a radioisotope, that is, e.g. a low-energy gamma photon emitter, has taken place. In certain embodiments such methods are particularly useful in localizing and removing secondary cancers produced by metastatic cells from a primary tumor.

The anti-ALPP/ALPPL2 antibodies described herein can be coupled directly to the radio-opaque moiety (e.g., at an available cysteine) or they can be attached to a “package” (e.g., a chelate, a liposome, a polymer microbead, a nanoparticle, etc.) carrying, containing, or comprising the radio-opaque material, e.g., as described below.

In addition to radio-opaque labels, other labels are also suitable for use. Detectable labels suitable for use in immunoconjugates include any composition detectable by spectroscopic, photochemical, biochemical, immunochemical, electrical, optical or chemical means. Useful labels in the include magnetic beads (e.g., DYNABEADS™) fluorescent dyes (e.g., fluorescein isothiocyanate, texas red, rhodamine, green fluorescent protein, and the like), radiolabels (e.g., H, I, S, C, or P), enzymes (e.g., horse radish peroxidase, alkaline phosphatase and others commonly used in an ELISA), and colorimetric labels such as colloidal gold or colored glass or plastic (e.g. polystyrene, polypropylene, latex, etc.) beads, nanoparticles, quantum dots, and the like.

In certain embodiments, suitable radiolabels include, but are not limited to ⁹⁹Tc, ⁹⁹Tc, ⁹⁷Ru, ⁹⁵Ru, ⁹⁴Tc, ⁹⁰Y, ⁹⁰Y, ⁸⁹Zr, ⁸⁶Y, ⁷⁷Br, ⁷⁷As, ⁷⁶Br, ⁷⁵Se, ⁷²As, ⁶⁸Ga, ⁶⁸Ga, ⁶⁷Ga, ⁶⁷Ga, ⁶⁷Cu, ⁶⁷Cu, ⁶⁴Cu, ⁶²Cu, ⁶²Cu, ⁵⁹Fe, ⁵⁸Co, ⁵⁷Co, ⁵²Mn, ⁵²Fe, ⁵¹Cr, ⁴⁷Sc, ³H, ³⁵S, ³³P, ³²P, ²²⁵Ac, ²²⁴Ac, ²²³Ra, ²¹³Bi, ²¹²Pb, ²¹²Bi, ²¹¹At, ²⁰³Pb, ²⁰³Hg, ²⁰¹Tl, ¹⁹⁹Au, ¹⁹⁸Au, ¹⁹⁸Au, ¹⁹⁷Pt, ¹⁸F, ¹⁸⁹Re, ¹⁸⁸Re, ¹⁸⁸Re, ¹⁸⁶Re, ¹⁸⁶Re, ¹⁷⁷Lu, ¹⁷⁷Lu, ¹⁷⁵Yb, ¹⁷²Tm, ¹⁶⁹Yb, ¹⁶⁹Yb, ¹⁶⁹Er, ¹⁶⁸Tm, ¹⁶⁷Tm, ¹⁶⁶Ho, ¹⁶⁶Dy, ¹⁶⁵Tm, ¹⁶⁵Dy, ¹⁶¹Tb, ¹⁵O, ¹⁵N, ¹⁵⁹Gd, ¹⁵⁹Gd, ¹⁵³Sm, ¹⁵³Pb, ¹⁵¹Pm, ¹⁴³Pr, ¹⁴²Pr, ¹³N, ¹³³I, ¹³¹In, ¹³¹I, ¹²⁷Te, ¹²⁶Te, ¹⁵⁸Te, ¹²⁵I, ¹²⁴I, ¹²³I, ¹²²Te, ¹²¹Te, ¹²¹Sn, ^{u}C, ¹³¹In, ^{U1}ln, ^{U1}ln, ^{lu}Ag, ^{lu}Ag, ¹⁰⁹Pd, ¹⁰⁹Pd, ¹⁰⁷Hg, ¹⁰⁵Ru, ¹⁰⁵Rh, ¹⁰⁵Rh, and ¹⁰³Ru.

Means of detecting such labels are well known to those of skill in the art. Thus, for example, certain radiolabels may be detected using photographic film, scintillation detectors, PET imaging, MRI, and the like. Fluorescent markers can be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

In another embodiment, the effector can comprise a radiosensitizer that enhances the cytotoxic effect of ionizing radiation (e.g., such as might be produced by ⁶⁰Co or an x-ray source) on a cell. Numerous radiosensitizing agents are known and include, but are not limited to benzoporphyrin derivative compounds (see, e.g., U.S. Pat. No. 5,945,439), 1,2,4-benzotriazine oxides (see, e.g., U.S. Pat. No. 5,849,738), compounds containing certain diamines (see, e.g., U.S. Pat. No. 5,700.825), BCNT (see, e.g., U.S. Pat. No. 5,872,107), radiosensitizing nitrobenzoic acid amide derivatives (see, e.g., U.S. Pat. No. 4,474,814), various heterocyclic derivatives (see, e.g., U.S. Pat. No. 5,064,849). platinum complexes (see, e.g., U.S. Pat. No. 4,921,963), and the like.

In certain embodiments, the effector can include an alpha emitter, i.e. a radioactive isotope that emits alpha particles. Alpha-emitters have recently been shown to be effective in the treatment of cancer (see, e.g., McDevitt et al. (2001) Science 294: 1537-1540; Ballangrud et al. (2001) Cancer Res. 61: 2008-2014; Borchardt et al. (2003) Cancer Res. 63: 5084-50). Suitable alpha emitters include, but are not limited to ²¹²Pb, ²²⁵Ac, ²²⁷Th, Bi, ²¹³Bi, ²¹¹At, and the like.

Many of the pharmaceuticals and/or radiolabels described herein can be provided as a chelate. The chelating molecule is typically coupled to a molecule (e.g. biotin, avidin, streptavidin, etc.) that specifically binds an epitope tag attached to an anti-ALPP/ALPPL2 antibody described herein.

Chelating groups are well known to those of skill in the art. In certain embodiments, chelating groups are derived from ethylene diamine tetra-acetic acid (EDTA), di ethylene triamine penta-acetic acid (DTP A), cyclohexyl 1,2-diamine tetra-acetic acid (CDTA), ethyleneglycol-0,0′-bis(2-aminoethyl)-N,N,N′,N′-tetra-acetic acid (EGTA), N,N-bis(hydroxybenzyl)-ethylenediamine-N,N′-diacetic acid (1HBED), triethylene tetramine hexa-acetic acid (TTHA), 1,4,7,10-tetraazacyclododecane-N,N′,N″,N′″-tetra-acetic acid (DOTA), hydroxy ethyldiamine triacetic acid (HEDTA), 1,4,8, 11-tetra-azacycl otetradecane-N,N′,N″,N′″-tetra-acetic acid (TETA), substituted DTP A, substituted EDTA, and the like.

Examples of certain chelators include unsubstituted or, substituted 2-iminothiolanes and 2-iminothiacyclohexanes, in particular 2-imino-4-mercaptomethylthiolane. One chelating agent, 1,4,7,10-tetraazacyclododecane-N,N′,N″,N′″-tetraacetic acid (DOTA), is of particular interest because of its ability to chelate a number of diagnostically and therapeutically important metals, such as radionuclides and radiolabels. Conjugates of DOTA and proteins such as antibodies have been described previously. For example, U.S. Pat. No. 5,428,156 teaches a method for conjugating DOTA to antibodies and antibody fragments. To make these conjugates, one carboxylic acid group of DOTA is converted to an active ester which can react with an amine or sulfhydryl group on the antibody or antibody fragment. Lewis et al. (1994) Bioconjugate Chem. 5: 565-576, describes a similar method wherein one carboxyl group of DOTA is converted to an active ester, and the activated DOTA is mixed with an antibody, linking the antibody to DOTA via the epsilon-amino group of a lysine residue of the antibody, thereby converting one carboxyl group of DOTA to an amide moiety.

In certain embodiments the chelating agent can be coupled, directly or through a linker, to an epitope tag or to a moiety that binds an epitope tag. Conjugates of DOTA and biotin have been described (see, e.g., Su (1995) J. N cl. Med., 36 (5 Suppl): 154P, which discloses the linkage of DOTA to biotin via available amino side chain biotin derivatives such as DOTA-LC-biotin or DOT A-benzyl-4-(6-amino-caproamide)-biotin). Yau et al, WO 95/15335, disclose a method of producing nitro-benzyl-DOTA compounds that can be conjugated to biotin. The method comprises a cyclization reaction via transient projection of a hydroxy group; tosylation of an amine; deprotection of the transiently protected hydroxy group; tosylation of the deprotected hydroxy group; and intramolecular tosylate cyclization. Wu et al. (1992) Nucl. Med. Biol., 19(2): 239-244 discloses a synthesis of macrocylic chelating agents for radiolabeling proteins with ¹¹¹IN and ⁹⁰Y. Wu et al. makes a labeled DOTA-biotin conjugate to study the stability and biodistribution of conjugates with avidin, a model protein for studies. This conjugate was made using a biotin hydrazide which contained a free amino group to react with an in situ generated activated DOTA derivative.

The anti-ALPP/ALPPL2 antibodies described herein can be used to deliver a variety of cytotoxic and/or cytostatic drugs including therapeutic drugs, a compound emitting radiation, cytotoxic molecules of plant, fungal, or bacterial origin, biological proteins, and mixtures thereof. In certain embodiments the cytotoxic drugs can comprise intracellularly acting cytotoxic drugs that are, e.g., small organic molecules, cytotoxic proteins or peptides, radiation emitters, including, for example, short-range, high-energy a-emitters as described above, and the like. Additional representative therapeutic agents include radioisotopes, chemotherapeutic agents, immunomodulatory agents, anti-angiogenic agents, anti-proliferative agents, pro-apoptotic agents, and cytolytic enzymes (e.g., RNases). An agent may also include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above-noted terms. For example, selected radioisotopes are also cytotoxins. In various embodiments therapeutic agents nay be prepared as pharmaceutically acceptable salts, acids or derivatives of any of the above.

In certain embodiments, the anti-ALPP/ALPPL2 antibody is attached to a therapeutic cytotoxic/cytostatic drug. In various embodiments the drugs being used to construct ADCs include, but are not limited to microtubule inhibitors and DNA-damaging agents, polymerase inhibitors (e.g., the polymerase II inhibitor, a-amanitin), and the like. In certain embodiments the antibody is conjugated to the drug directly or through a linker, while in other embodiments, the antibody is conjugated to a drug carrier {e.g., a liposome containing the drug, a polymeric drug carrier, a nanoparticle drug carrier, a lipid drug carrier, a dendrimeric drug carrier, and the like).

In certain embodiments the drug comprises a tubulin inhibitor, including, but not limited to auristatin, Dolastatin-10, synthetic derivatives of the natural product Dolastatin-10, and maytansine or a maytansine derivative. In certain embodiments the drug comprises an auristatin. In certain embodiments the auristatin is selected from the group consisting of auristatin E (AE), auristatin EB (AEB), auristatin EFP (AEFP). Monomethyl Auristatin D (MMAD) or monomethyl dolastatin 10, Monomethyl Auristatin F (MMAF) or N-methylvaline-valine-dolaisoleuine-dolaproine-phenylalanine), Monomethyl Auristatin E (MMAE) or N-methylvaline-valine-dolaisoleuine-dolaproine-norephedrine, 5-benzoyl valeric acid-AE ester (AEVB), vcMMAE, and vcMMAF.

In certain embodiments the drug comprises an enediyne. Enediynes are a class of anti-tumor bacterial products characterized by either nine- and ten-membered rings or the presence of a cyclic system of conjugated triple-double-triple bonds. Exemplary enediynes include, but are not limited to, calicheamicin, esperamicin, and dynemicin.

Calicheamicin is an enediyne antibiotic that was originally isolated as a natural product from the soil organism Micromonospora echinospora ssp. calichensis (Zein et al. Science 27; 240(4856): 1198-1201, 1988). It generates double-strand DNA breaks and subsequently induces apoptosis in target cells (Zein et al. Science 27; 240(4856): 1198-1201, 1988; Nicolaou et al. Chem. Biol. September; l(l):57-66, 1994; Prokop et al. Oncogene 22:9107-9120, 2003). In certain embodiments the drug comprises calicheamicin or a calicheamicin analog. Examples of calicheamicins and analogs thereof suitable for use anti-ALPPL2 immunoconjugates are disclosed, for example, in U.S. Pat. Nos. 4,671,958 4,970,198, 5,053,394, 5,037,651, 5,079,233, 5,264,586, and 5,108,912, which are incorporated herein by reference in their entirety. In certain embodiments these compounds contain a methyltrisulfide that can be reacted with appropriate thiols to form disulfides, at the same time introducing a functional group such as a hydrazide or other functional group that is useful for conjugating calicheamicin to an anti-ALPPL2 antibody. Disulfide analogs of calicheamicin can also be used, for example, analogs described in U.S. Pat. Nos. 5,606,040 and 5,770,710, which are incorporated herein by reference in its entirety. In certain embodiments the disulfide analog is N-acetyl-gamma-calicheamicin dimethyl hydrazide.

In certain embodiments the drug comprises a geldanamycin. Geldanamycins are benzoquinone ansamycin antibiotic that bind to H1sp90 (Heat Shock Protein 90) and have been used antitumor drugs. Exemplary geldanamycins include, but are not limited to, 17-AAG (17-N-Allylamino-17-Demethoxygeldanamycin), and 17-DMAG (17-Dimethylaminoethylamino-17-demethoxygeldanamycin). In certain embodiments the drug comprises a maytansine. Maytansines or their derivatives maytansinoids inhibit cell proliferation by inhibiting the microtubules formation during mitosis through inhibition of polymerization of tubulin (see, e.g., Remillard et al. 91975) Science 189: 1002-1005). Illustrative maytansines include, but are not limited to, Mertansine (DM1); and an analogue of maytansine such as DM3 or DM4, as well as ansamitocin.

In certain embodiments the drug comprises a taxane. Taxanes are diterpenes that act as anti-tubulin agents or mitotic inhibitors. Exemplary taxanes include, but are not limited to, paclitaxel and docetaxel.

In certain embodiments the drug comprises a DNA interacting agent. In certain embodiments the DNA interacting agent includes, but is not limited to calicheamicins, duocarmycins, pyrrolobenzodiazepines (PBDs), and the like.

In another illustrative, but non-limiting embodiment, the drug comprises a duocarmycin. Duocarmycins are DNA damaging agents able to exert their mode of action at any phase in the cellular cycle. Agents that are part of this class of duocarmycins typically have potency in the low picomolar range. Illustrative duocarmycins {e.g., duocarmycin analogues) that can be used as effectors in the chimeric constructs contemplated herein include, but are not limited to duocarmycin A, duocarmycin B1, duocarmycin B2, duocarmycin C1, duocarmycin C2, duocarmycin D, duocarmycin SA, Cyclopropylbenzoindole duocarmycin (CC-1065), Centanamycin, Rachelmycin,

In another illustrative, but non-limiting embodiment, the drug comprises a pyrrolobenzodiazepine. In certain embodiments the drug comprises a synthetic derivative of two pyrrolobenzodiazepines linked by a flexible polymethylene tether. Pyrrolobenzodiazepines (PBDs) and PBD dimers are described in U.S. Pat. No. 7,528,126 B2, which is incorporated herein by reference for the Pyrrolobenzodiazepines and PBD dimers described therein. In certain embodiments the pyrrolobenzodiazepine is selected from the group consisting of: Anthramycin (and dimers thereof), Mazethramycin (and dimers thereof), Tomaymycin (and dimers thereof). Prothracarcin (and dimers thereof), Chicamycin (and dimers thereof), Neothramycin A (and dimers thereof), Neothramycin B (and dimers thereof), DC-81 (and dimers thereof), Sibiromycin (and dimers thereof), Porothramycin A (and dimers thereof), Porothramycin B (and dimers thereof), Sibanomycin (and dimers thereof), Abbeymycin (and dimers thereof), SG2000, and SG2285.

In certain embodiments the drug comprise a polymerase inhibitor, including, but not limited to polymerase II inhibitors such as α-amanitin, and poly(ADP-ribose) polymerase (PARP) inhibitors. Illustrative PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, 3-aminobenzamide, and the like.

In certain embodiments the drug comprises a vinca alkyloid. Vinca alkyloids are also anti-tubulin agents. Exemplary vinca alkyloids include, but are not limited to, vincristine, vinblastine, vindesine, and vinorelbine.

The foregoing drugs are illustrative and not limiting. In various embodiments other anti-cancer drugs can be utilized including but not limited to anti-cancer antibodies (e.g., HERCEPTIN®), antimetabolites, alkylating agents, topoisomerase inhibitors, microtubule targeting agents, kinase inhibitors, protein synthesis inhibitors, somatostatin analogs, glucocorticoids, aromatose inhibitors, mTOR inhibitors, protein Kinase B (PKB) inhibitors, phosphatidylinositol, 3-Kinase (PI3K) Inhibitors, cyclin dependent kinase inhibitors, anti-TRAIL molecules, MEK inhibitors, and the like. In certain embodiments the anti-cancer compounds include, but are not limited to flourouracil (5-FU), capecitabine/XELODA, 5-Trifluoromethyl-2′-deoxyuridine, methotrexate sodium, raltitrexed/Tomudex, pemetrexed/Alimta®, cytosine Arabinoside (Cytarabine, Ara-C)/Thioguanine, 6-mercaptopurine (Mercaptopurine, 6-MP), azathioprine/Azasan, 6-thioguanine (6-TG)/Purinethol (TEVA), pentostatin/Nipent, fludarabine phosphate/Fludara®, cladribine (2-CdA, 2-chlorodeoxyadenosine)/Leustatin, floxuridine (5-fluoro-2)/FUDR (Hospira, Inc.), ribonucleotide Reductase Inhibitor (RNR), cyclophosphamide/Cytoxan (BMS), neosar, ifosfamide/Mitoxana, thiotepa, BCNU-1,3-bis(2-chloroethyl)-1-nitosourea, 1.-(2-chloroethyl)-3-cyclohexyl-lnitrosourea, methyl CCNU. hexamethylmelamine, busulfan/Myleran, procarbazine HCL/Matulane, dacarbazine (DTIC), chlorambucil/Leukaran ., melphalan/l keran, cisplatin (Cisplatinum, CDDP)/Platinol, carboplatin/Paraplatin, oxaliplatin/Eloxitan, bendamustine, carmustine, chloromethine, dacarbazine (DTIC), fotemustine, lomustine, mannosulfan, nedaplatin, nimustine, prednimustine, ranimustine, satraplatin, semustine, streptozocin, temozolomide, treosulfan, triaziquone, triethylene melamine, thioTEPA, triplatin tetranitrate, trofosfamide, uramustine, doxorubicin HCL/Doxil, daunorubicin citrate/Daunoxome®, mitoxantrone HCL/Novantrone, actinomycin D, etoposide/Vepesid, topotecan HCL/Hycamtin, teniposide (VM-26), irinotecan HCL(CPT-11)/, Camptosar®, camptothecin, Belotecan, rubitecan, vincristine, vinblastine sulfate, vinorelbine tartrate, vindesine sulphate, paclitaxel/Taxol. docetaxel/Taxotere, nanoparticle paclitaxel, abraxane, ixabepilone, larotaxel, ortataxel, tesetaxel, vinflunine, and the like. In certain embodiments the anti-cancer drug(s) comprise one or more drugs selected from the group consisting of carboplatin(e.g., PARAPLATIN®), Cisplatin (e.g., PLATINOL®, PLATINOL-AQ®), Cyclophosphamide (e.g., CYTOXAN®, NEOSAR®), Docetaxel (e.g., TAXOTERE®), Doxorubicin (e.g., ADRIAMYCIN®), Erlotinib (e.g., TARCEVA®), Etoposide (e.g., VEPESID®), Fluorouracil (e.g., 5-FU®), Gemcitabine (e.g., GEMZAR( ), imatinib mesylate (e.g., GLEEVEC®), Irinotecan (e.g., CAMPTOSAR®), Methotrexate (e.g., FOLEX®, MEXATE®, AMETHOPTERIN®), Paclitaxel (e.g., TAXOL®9, ABRAXANE®), Sorafmib (e.g., NEXAVAR®), Sunitinib (e.g., SUTENT®), Topotecan (e.g., HYCAMTIN®), Vinblastine (e.g., VELBAN®), Vincristine (e.g., ONCOVIN®, VINCASAR PFS®). In certain embodiments the anti-cancer drug comprises one or more drugs selected from the group consisting of retinoic acid, a retinoic acid derivative, doxirubicin, vinblastine, vincristine, cyclophosphamide, ifosfamide, cisplatin, 5-fluorouracil, a camptothecin derivative, interferon, tamoxifen, and taxol. In certain embodiments the anti-cancer compound is selected from the group consisting of abraxane, doxorubicin, pamidronate disodium, anastrozole, exemestane, cyclophosphamide, epirubicin, toremifene, letrozole, trastuzumab, megestroltamoxifen, paclitaxel, docetaxel, capecitabine, goserelin acetate, zoledronic acid, vinblastine, etc.), an antisense molecule, an SiRNA, and the like.

In certain embodiments the cytotoxic/cytostatic agent comprises a protein or peptide toxin or fragment thereof. Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, a-sacrin. certain Aleurites for dii proteins, certain Dianthin proteins, Phytolacca americana proteins (PAP, PAPII and PAP-S), Morodica charantia inhibitor, curcin, crotin, Saponaria officinalis inhibitor. gelonin, mitogillin, restrictocin, phenomycin, enomycin, and the tricothecenes, for example.

In certain embodiments the cytotoxins can include, but are not limited to Pseudomonas exotoxins, Diphtheria toxins, ricin, abrin and derivatives thereof Pseudomonas exotoxin A (PE) is an extremely active monomelic protein (molecular weight 66 kD), secreted by Pseudomonas aeruginosa, which inhibits protein synthesis in eukaryotic cells through the inactivation of elongation factor 2 (EF-2) by catalyzing its ADP-ribosylation (catalyzing the transfer of the ADP ribosyl moiety of oxidized NAD onto EF-2).

The toxin contains three structural domains that act in concert to cause cytotoxicity. Domain la (amino acids 1-252) mediates cell binding. Domain II (amino acids 253-364) is responsible for translocation into the cytosol and domain TTT (amino acids 400-613) mediates ADP ribosylation of elongation factor 2, which inactivates the protein and causes cell death. The function of domain lb (amino acids 365-399) remains undefined, although a large part of it, amino acids 365-380, can be deleted without loss of cytotoxicity. See Siegall et al. (1989) J. Biol. Chem. 264: 14256-14261.

In certain embodiments the antibody is attached to a preferred molecule in which domain la (amino acids 1 through 252) is deleted and amino acids 365 to 380 have been deleted from domain lb. In certain embodiments all of domain lb and a portion of domain 11 (amino acids 350 to 394) can be deleted, particularly if the deleted sequences are replaced with a linking peptide.

In addition, the PE and other cytotoxic proteins can be further modified using site-directed mutagenesis or other techniques known in the art, to alter the molecule for a particular desired application. For example, means to alter the PE molecule in a manner that does not substantially affect the functional advantages provided by the PE molecules described here can also be used and such resulting molecules are intended to be covered herein.

Methods of cloning genes encoding PE fused to various ligands are well known to those of skill in the art (see. e.g., Siegall et al (1989) FASEB J., 3: 2647-2652; and Chaudhary et al. (1987) Proc. Natl. Acad. Sci. USA, 84: 4538-4542).

Like PE, diphtheria toxin (DT) kills cells by ADP-ribosylating elongation factor 2 thereby inhibiting protein synthesis. Diphtheria toxin, however, is divided into two chains, A and B, linked by a disulfide bridge. In contrast to PE, chain B of DT, which is on the carboxyl end, is responsible for receptor binding and chain A, which is present on the amino end, contains the enzymatic activity (Uchida et al. (1972) Science, 175: 901-903; Uchida et al. (1973) J. Biol. Chem., 248: 3838-3844).

In certain embodiments, the antibody-Diphtheria toxin immunoconjugates of have the native receptor-binding domain removed by truncation of the Diphtheria toxin B chain. One illustrative modified Dipththeria toxin is DT388, a DT in which the carboxyl terminal sequence beginning at residue 389 is removed (see, e.g., Chaudhary et al. (1991) Bioch. Biophys. Res. Comm., 180: 545-551). Like the PE chimeric cytotoxins, the DT molecules can be chemically conjugated to the anti-ALPP antibody, but, in certain preferred embodiments, the antibody will be fused to the Diphtheria toxin by recombinant means (see, e.g., Williams et al. (1990) J. Biol. Chem. 265: 11885-11889).

In certain embodiments the anti-ALPP/ALPPL2 antibodies are attached to an immunomodulatory and function to localize the immunomodulatory at the cancer cell/tumor site. Numerous immunomodulators that can activate an immune response are known to those of skill in the art. In one illustrative, but non-limiting embodiment the immunomodulatory comprise an anti-CD3 antibody. Anti-CD3 monoclonal antibodies induce the proliferation of human T-cells cells in vitro and activate specific and nonspecific cytolysis by human T-cell clones and human peripheral blood lymphocytes. In vivo administration of anti-CD3 prevents tumor growth of a UV-induced mouse fibro sarcoma.

In certain embodiments the immunomodulators comprise agents that blockade immune checkpoints. Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. It is now clear that tumors co-opt certain immune-checkpoint pathways as a major mechanism of immune resistance, particularly against T cells that are specific for tumor antigens. Because many of the immune checkpoints are initiated by ligand-receptor interactions, they can be readily blocked by antibodies or modulated by recombinant forms of ligands or receptors.

Cytotoxic T-lymphocyte-associated antigen 4 (CTLA4) antibodies were the first of this class of immunotherapeutics to achieve US Food and Drug Administration (FDA) approval. The first such drug to receive approval, ipilimumab (Yervoy®), for the treatment of advanced melanoma, blocks the activity of a checkpoint protein known as CTLA4, which is expressed on the surface of activated immune cells called cytotoxic T lymphocytes. CTLA4 acts as a “switch” to inactivate these T cells. thereby reducing the strength of immune responses; ipilimumab binds to CTLA4 and prevents it from sending its inhibitory signal. Two other FDA-approved checkpoint inhibitors, nivolumab (Opdivo®) and pembrolizumab (Keytruda®), work in a similar way, but they target a different checkpoint protein on activated T cells known as PD-1. Nivolumab is approved to treat some patients with advanced melanoma or advanced lung cancer, and pembrolizumab is approved to treat some patients with advanced melanoma. Accordingly in certain embodiments the immunomodulators comprise antibodies directed against CTLA4 (e.g., ipilimumab), and/or antibodies directed against PD-L1 (e.g., nivolumab, pembrolizumab), and/or antibodies directed against PD-L2.

Other examples of immune modulators that can be attached to the anti-ALPP/ALPPL2 antibody include, but are not limited to, gancyclovier, etanercept, tacrolimus, sirolimus. voclosporin, cyclosporine, rapamycin, cyclophosphamide, azathioprine, mycophenolgate mofetil, methotrextrate, glucocorticoid and its analogs, cytokines, xanthines, stem cell growth factors, lymphotoxins, tumor necrosis factor (TNF), hematopoietic factors, interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S 1 factor,” erythropoietin and thrombopoietin, or a combination thereof.

Useful immunomodulatory agents also include anti-hormones that block hormone action on tumors and immunosuppressive agents that suppress cytokine production, down-regulate self-antigen expression, or mask MHC antigens. Representative anti-hormones include anti-estrogens including, for example, tamoxifen. raloxifene, aromatase inhibiting 4(5)-imidazoles, 4-hydroxytamoxifen, trioxifene, keoxifene, LY 1 17018, onapnstone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, cytokine or cytokine receptor antagonists (e.g., anti-interferon antibodies, anti-ILlO antibodies, anti-TNFa antibodies, anti-1L2 antibodies), streptokinase, TGFP, rapamycin, T-cell receptor, T-cell receptor fragments, and T cell receptor antibodies.

In certain embodiments, the effector comprises a viral particle (e.g., a filamentous phage, an adeno-associated virus (AAV), a lentivirus, and the like). The antibody can be conjugated to the viral particle and/or can be expressed on the surface of the viral particle (e.g. a filamentous phage). The viral particle can additionally include a nucleic acid that is to be delivered to the target (e.g., a cancer cell that expresses ALPPL2) cell. The use of viral particles to deliver nucleic acids to cells is described in detail in WO 99/55720, U.S. Pat. Nos. 6,670,188, 6,642,051, and 6,669,936.

In certain embodiments, the anti-ALPP/ALPPL2 antibodies described herein can be chemically conjugated to the effector molecule (e.g., a cytotoxin, a label, a ligand, a drug, a liposome, etc.). Means of chemically conjugating molecules are well known to those of skill. The procedure for attaching an effector to an antibody will vary according to the chemical structure of the effector and/or antibody. Polypeptides typically contain variety of functional groups; e.g., carboxylic acid (COOH) or free amine (—H2) groups, that are available for reaction with a suitable functional group on an effector molecule to bind the effector thereto. Alternatively, the antibody and/or the effector can be derivatized to expose or attach additional reactive functional groups. The derivatization can involve attachment of any of a number of linker molecules such as those available from Pierce Chemical Company, Rockford Illinois.

A “linker”, as used herein, is a molecule that is used to join the targeting molecule to the effector molecule. The linker is capable of forming covalent bonds to both the targeting molecule and to the effector molecule. Suitable linkers are well known to those of skill in the art and include, but are not limited to, straight or branched-chain carbon linkers, heterocyclic carbon linkers, or peptide linkers. Where the targeting molecule and the effector molecule are polypeptides, the linkers may be joined to the constituent amino acids through their side groups (e.g., through a disulfide linkage to cysteine). However, in a preferred embodiment, the linkers will be joined to the alpha carbon amino or carboxyl groups of the terminal amino acids.

The immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. (1987) Science 238: 1098. Carbon-14-labeled 1-isothiocyanatobenzyl-3-methyldiethylene triaminepentaacetic acid (MX-DTPA) is an illustrative, but non-limiting, chelating agent for conjugation of, e.g., a radionucleotide to the antibody (see, e.g., WO 1994/011026 (PCT/US 1993/010953)).

In certain embodiments conjugation of effectors (e.g., drugs, liposomes, etc.) or linkers attached to effectors, to an antibody takes place at solvent accessible reactive amino acids such as lysines or cysteines that can be derived from the reduction of inter-chain disulfide bonds in the antibody. In certain embodiments cysteine conjugation can occur after reduction of four inter-chain disulfide bonds.

The disclosure also provides bispecific antibodies comprising a variable region as described herein that specifically binds ALPPL2 and ALPP and a second variable region that specifically binds to surface marker on a peripheral blood mononuclear cell (PBMC), for example CD3. For example, a bispecific antibody can be constructed by combining anti-ALPPL2 and ALPP and anti-T cell (e.g., CD3) antibody fragments using any known bispecific antibody configuration. Examples include but are not limited to the BiTE (Bispecific T Cell Engager) (Harrington et al. (2015) PloS One 10: e0135945; Klinger et al. (2012) Blood, 119: 6226-6233; Molhoj et al. (2007) Mol. Immunol. 44: 1935-1943), diabodies, or DART (Dual-Affinity Retargeting) platforms (Chi chili et al (2015) Sci. Transl. Med. 7: 289ra282; Moore et al. (2011) Blood, I17: 4542-4551). The BiTE (bispecific T cell engager) molecules have been very well characterized and already shown promise in the clinic (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)). The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

In certain embodiments, diabodies comprising one or more of the VH and VL domains described herein are contemplated. The term “diabodies” refers to antibody fragments typically having two antigen-binding sites. The fragments typically comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161, and Holliger et al. (1993) Proc. Natl. Acad. Sci. USA 90: 6444-6448.

In some embodiments, the anti-CD3 variable region of a bispecific antibody comprises a heavy chain variable region CDR1, CDR2, and CDR3 comprising SEQ ID NO: 79. SEQ ID NO: 80 and SEQ ID NO: 81, respectively, and a light chain variable region comprising CDR1, CDR2, and CDR3 comprising SEQ ID NO: 82, SEQ ID NO: 83 and SEQ ID NO: 84, respectively. In some embodiments, the bispecific antibody comprises a heavy chain variable region comprising SEQ ID NO: 77, a light chain variable region comprising SEQ ID NO:78, or both. Non-limiting exemplary bispecific antibodies comprise one or both of SEQ ID NO:85 and SEQ ID NO:86 or one or both of SEQ ID NO:87 and SEQ ID NO:88.

Also provided are cells expressing a CAR that comprises an ALPPL2 and ALPP-binding domain from an antibody as described herein. Chimeric antigen receptors (CARs) are recombinant receptor constructs comprising an extracellular antigen-binding domain (e.g., a ALPPL2 and ALPP-binding domain from an antibody as described herein) joined to a transmembrane domain, and further linked to an intracellular signaling domain (e.g., an intracellular T cell signaling domain of a T cell receptor) that transduces a signal to elicit a function. In certain embodiments, immune cells (e.g., T cells or natural killer (NK) cells or macrophages) are genetically modified to express CARs that comprise one or more ALPPL2 and ALPP-binding domains of the antibodies described herein and have the functionality of effector cells (e.g., T cell cytotoxic functions).

In some standard CAR embodiments, the components include an extracellular targeting domain comprising an ALPPL2 and ALPP-variable region as described herein, a transmembrane domain and intracellular signaling/activation domain, which are typically linearly constructed as a single fusion protein. The “transmembrane domain” is the portion of the CAR that links the extracellular binding portion and intracellular signaling domain and anchors the CAR to the plasma membrane of the host cell that is modified to express the CAR, e.g., the plasma membrane of an immune effector cell. The intracellular region may contain a signaling domain of TCR complex, and/or one or more costimulatory signaling domains, such as those from CD28, 4-1BB (CD137) and OX-40 (CD134). For example, a “first-generation CAR” generally has a CD3-zeta signaling domain. Additional costimulatory intracellular domains may also be introduced (e.g., second and third generation CARS) and further domains including homing and suicide domains may be included in CAR constructs. CAR components are further described below.

A CAR construct encoding a CAR may also comprise a sequence that encodes a signal peptide to target the extracellular domain to the cell surface.

In some embodiments, the CAR may contain one or more hinge domains that link the antigen binding domain comprising the anti-ALPPL2 and ALPP-binding domain and the transmembrane domain for positioning the antigen binding domain. Such a hinge domain may be derived either from a natural, synthetic, semi-synthetic, or recombinant source. The hinge domain can include the amino acid sequence of a naturally occurring immunoglobulin hinge region, e.g., a naturally occurring human immunoglobulin hinge region, or an altered immunoglobulin hinge region. Illustrative hinge domains suitable for use in the CARs described herein include the hinge region derived from the extracellular regions of type 1 membrane proteins such as CD8 alpha, CD4, CD28, PD1, CD 152, and CD7, which may be wild-type hinge regions from these molecules or may be altered.

Any transmembrane suitable for use in a CAR construct may be employed. Such transmembrane domains, include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD 18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100, (SEMA4D), SLAMF6 (NTB-A, Lyl08), SLAM (SLAMF1, CD150, IPO-3), BLAME, (SLAMF8), SELPLG (CD162), LTBR, PAG/Cbp, NKG2D, or NKG2C.

A transmembrane domain incorporated into a CAR construct may be derived either from a natural, synthetic, semi-synthetic, or recombinant source.

A CAR construct of the present disclosure includes one or more intracellular signaling domains, also referred to herein as co-stimulatory domains, or cytoplasmic domains that activate or otherwise modulate an immune cell, (e.g., a T lymphocyte). The intracellular signaling domain is generally responsible for activation of at least one of the normal effector functions of the immune cell in which the CAR has been introduced. In one embodiment, a co-stimulatory domain is used that increases CAR immune T cell cytokine production. In another embodiment, a co-stimulatory domain is used that facilitates immune cell (e.g., T cell) replication. In still another embodiment, a co-stimulatory domain is used that prevents CAR immune cell (e.g., T cell) exhaustion. In another embodiment, a co-stimulatory domain is used that increases immune cell (e.g., T cell) antitumor activity. In still a further embodiment, a co-stimulatory domain is used that enhances survival of CAR immune cells (e.g., T cells) (e.g., post-infusion into patients).

Examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains include those of CD3 zeta, common FcR gamma, Fc gamma Rlla, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

An intracellular signaling domain of a CAR can comprise a primary intracellular signaling domain only, or may comprise additional desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Lyl08), SLAM, (SLAMF1, CD150, IPO-3), BLAME (SLAMF8), SELPLG (CD162), LTBR, LAT, GADS, SLP-76, PAG/Cbp, and CD 19a.

In some embodiments, a CAR may be designed as an inducible CAR, or may otherwise comprise a mechanism for reversibly expressing the CAR, or controlling CAR activity to largely restrict it to a desired environment. Thus, for example, in some embodiments, the CAR-expressing cell uses a split CAR. The split CAR approach is described in more detail in publications WO2014/055442 and WO2014/055657.

In some embodiments, a cell expressing a CAR comprising one or more ALPPL2 and ALPP-binding domains as described herein also expresses a second CAR, e.g., a second CAR that includes a different antigen binding domain, e.g., that binds to the same target or a different target.

In some embodiments, a host cell, e.g., a host T cell is modified to express a ALPPL2 and ALPP-binding domain as described herein, or a chimeric molecules, such as a chimeric receptor comprising such a domain, using a gene editing system, such as a Cas/CRISPR system, a Transcription activator-like effector nuclease (TALEN) system, a homing endonuclease (HE) system, or a zinc-finger nuclease (ZFN) system.

Many methods for introducing nucleic acids and viral vectors (e.g., viral particles) into a target cell (e.g., a CD8⁺ T cell) are available. Non-limiting examples of suitable methods include electroporation (e.g., nucleofection), viral or bacteriophage infection, transfection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, microparticle- or nanoparticle-mediated nucleic acid delivery, and the like. In some embodiments, a viral vector may be used, such as an adenovirus, adeno-associated virus (AAV), lentivirus vector, a vaccinia virus vector, or any of a number of different vectors. In some

The invention is not limited by the type of immune cells genetically modified to express a CAR. Illustrative immune cells include, but are not limited to, T cells, e.g., alpha/beta T cells and gamma/delta T cells, B cells, natural killer (NK) cells, natural killer T (NKT) cells, mast cells, macrophages, and myeloid-derived phagocytes. In some embodiments, the T cells are CD8+ T cells Treg cells. In some embodiments, the immune cells, e.g., T cells, are autologous cells from the patient to undergo immunotherapy. In some embodiments, the immune cells are allogeneic. Methods of making CAR-expressing cells are described, e.g., in US2016/0185861 and US2019/0000880.

The antibodies described herein as well as the cells expressing a CAR as described herein can be used to treat cancer, i.e., cancer cells that express ALPPL2 or ALPP or both. Cancers that may be treated include tumors that are not vascularized, or not yet substantially vascularized, as well as vascularized tumors. The cancers may comprise non-solid tumors or may comprise solid tumors, or may comprise cancer cells (e.g., cancer stem cells). Types of cancers to be treated with the antibodies or CARs described herein include, but are not limited to mesothelioma, testicular cancer, endometrial cancer, and subsets of ovarian, pancreatic, and non-small cell lung cancers.

Antibodies described herein (including ALPPL2 and ALPP binding fragments thereof, affinity matured variants, or scFvs) can be used for diagnosis, either in vivo or in vitro (e.g., using a biological sample obtained from an individual). In some embodiments, for example, the method allows for detection of mesothelioma, testicular cancer, endometrial cancer, and subsets of ovarian, pancreatic, or non-small cell lung cancer.

When used for detection or diagnosis, the antibody is typically conjugated or otherwise associated with a detectable label. The association can be direct e.g., a covalent bond, or indirect, e.g., using a secondary binding agent, chelator, or linker.

A labeled antibody can be provided to an individual to determine the applicability of an intended therapy. For example, a labeled antibody may be used to detect ALPPL2 and/or ALPP expression or density within a diseased area. For therapies intended to target ALPPL2 and ALPP, the density of p ALPPL2 and/or ALPP is typically high relative to non-diseased tissue. A labeled antibody can also indicate that the diseased area is accessible for therapy. Patients can thus be selected for therapy based on imaging results. Anatomical characterization, such as determining the precise boundaries of a cancer, can be accomplished using standard imaging techniques (e.g., CT scanning, MRI, PET scanning, etc.). Such in vivo methods can be carried out using any of the presently disclosed antibodies.

Any of the presently disclosed antibodies can also be used for in vitro diagnostic or monitoring methods, e.g., using cells or tissue from a patient sample. In some embodiments, labeled antibodies as described herein are used, as it can bind fixed cells as well as non-fixed cells.

A diagnostic agent comprising an antibody described herein can include any diagnostic agent known in the art, as provided, for example, in the following references: Armstrong et al., Diagnostic Imaging, 5^{th} Ed., Blackwell Publishing (2004); Torchilin, V. P. Ed., Targeted Delivery of Imaging Agents, CRC Press (1995); Vallabhajosula, S., Molecular Imaging: Radiopharmaceuticals for PET and SPECT, Springer (2009). The terms “detectable agent,” “detectable moiety,” “label,” “imaging agent,” and like terms are used synonymously herein. A diagnostic agent can be detected by a variety of ways, including as an agent providing and/or enhancing a detectable signal. Detectable signals include, but are not limited to, gamma-emitting, radioactive, echogenic, optical, fluorescent, absorptive, magnetic, or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like. PET is particularly sensitive and quantitative, and thus valuable for characterizing processes in vivo (Olafsen et al. (2012) Tumour Biol. 33:669-77; Cai et al. (2007) J Nucl Med. 48:304-10). This is useful beyond a companion diagnostic and would be generally useful to diagnose, clinically stage and follow patients during any treatment regimen. Methods of detection involving one or more antibody as described herein can include, but are not limited to, e.g., ELISA, electrochemiluminescence immunoassay (ECLIA), western blot analysis, radioimmunoassay, immunofluorimetry, immunoprecipitation, equilibrium dialysis, immunodiffusion, a solution phase assay, or immunohistochemical or other methods.

Pharmaceutical compositions comprising an antibody as described herein or a cell expressing a CAR as described herein can include one or more pharmaceutically acceptable carriers. Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. Pharmaceutical compositions may be administered parenterally in the form of an injectable formulation. Pharmaceutical compositions for injection (i.e., intravenous injection) can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

The pharmaceutical composition may be formed in a unit dose form as needed. The amount of active component, e.g., an antibody as described herein, included in the pharmaceutical preparations is such that a suitable dose within the designated range is provided (e.g., a dose within the range of 0.01-500 mg/kg of body weight).

Pharmaceutical compositions described herein may be formulated for subcutaneous administration, intramuscular administration, intravenous administration, parenteral administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. In some embodiments, pharmaceutical compositions may administered locally or systemically (e.g., locally). In particular embodiments, pharmaceutical compositions may be administered locally at the affected area, such as skin or cancerous tissue.

The dosage of the pharmaceutical compositions depends on factors including the route of administration, the disease to be treated, and physical characteristics, e.g., age, weight, general health, of the subject. In some embodiments, the amount of active ingredient (e.g., an antibody as described herein) contained within a single dose are administered in an amount that effectively prevents, delays, or treats the disease without inducing significant toxicity. The dosage may be adapted by the physician in accordance with conventional factors such as the extent of the disease and different parameters of the subject.

The pharmaceutical compositions may be administered in a manner compatible with the dosage formulation and in such amount as is therapeutically effective to result in an improvement or remediation of the symptoms. The pharmaceutical compositions may be administered in a variety of dosage forms, e.g., subcutaneous dosage forms, intravenous dosage forms, and oral dosage forms (e.g., ingestible solutions, drug release capsules). Pharmaceutical compositions containing the active ingredient (e.g., an antibody as described herein) may be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines.

In determining the effective amount of the antibodies to be administered, a physician may evaluate circulating plasma levels of the antibody and antibody toxicity. In general, the dose equivalent of an antibody is from about 1 ng/kg to 10 mg/kg for a typical subject. In some embodiments, the dose range for sub-cutaneous or iv administration is 0.1-20, e.g., 0.3-3 mg/kg.

### EXAMPLE

### Example 1

The high specificity of ALPPL2 allows development of additional therapeutics beyond ADC, including bispecific immune effector cell engager that has a different tumor killing mechanism. We thus sought to develop and characterize an ALPPL2-targeted bispecific T cell engager. The original M25 antibody is fully human in sequence and binds to ALPPL2 expressing cells with sub nM to low nM apparent binding affinity in the IgG1 form (Su et al, 2020). The monovalent binding of M25 single chain fragment variable (scFv) or Fab to ALPPL2 is moderate/low. We have previously performed affinity maturation study and identify a high affinity variant of M25, M25FYIA (aka FYIA), with over 100-fold improvement in monovalent binding to ALPPL2 over the parental M25 (Su et al, 2020; Liu et al, 2017). Most importantly, FYIA retains high specificity, binding only to ALPPL2/ALPP but not the closely related ALPI (Su, Y., et al. Cancer Res., 2020 Aug. 31; WO2017095823).

In this study, in the context of bispecific T cell engager development, FYIA was further optimized to increase monovalent binding affinity to ALPPL2 while improving developability characteristics (avoiding charged or hydrophobic patches and removal of deamidation and isomerization motifs). The framework regions of FYIA were modified to match germline sequences (IGHV3-23*04 and IGLV2-14*01) to create FYIA_germ. Using FYIA_germ as a starting point, a combination of site-directed and error-prone PCR mutagenesis was used to generate clones that were selected or screened for improved binding to human ALPPL2 and no binding to human ALPI. A panel of affinity- and developability-optimized human antibodies was identified (VH and VL sequences are shown in Tables 1 and 2, respectively). The lead antibody in this panel, FYIAgermopt aka SYLY, was selected for bispecific antibody construction.

TABLE 1
Heavy chain variable sequences

| | | | CDR | |
|---|---|---|---|---|
| SEQ | | | SEQ | |
| ID | | | ID | CDR (1, 2, 3, |
| NO: | Name | VH sequence | NO: | respectively) |
| 1 | FYIA | EVQLVESGGGLVQPGGSLRLSCAASGFTF | 20 | AASGFTESSYAMH |
| | germ | SSYAMHWVRQAPGKGLEWVAVISYDGSNK | 21 | VISYDGSNKY |
| | _HC | YYADSVKGRFTISRDNSKNTLYLQMNSLR | 22 | AKEGDSSRWSYDL |
| | | AEDTAVYYCAKEGDSSRWSYDLWGRGTLV | | |
| | | TVSS | | |
| 2 | FYIA | EVQLVESGGGLVQPGGSLRLSCAASGFTF | 23 | AASGFTFSAYAMH |
| | germ | SAYAMHWVRQAPGKGLEWVAVISYEGYNA | 24 | VISYEGYNAY |
| | _SYLY | YYADSVKGRFTISRDNSKNTLYLQMNSLR | 25 | AKEGDYSRWAYDY |
| | | AEDTAVYYCAKEGDYSRWAYDYWGRGTLV | | |
| | | TVSS | | |
| 3 | FYIA | EVQLVESGGGLVQPGGSLRLSCAASGFTF | 26 | AASGFTFSAYAMH |
| | germ | SAYAMHWVRQAPGKGLEWVAVISYEGYNA | 27 | VISYEGYNAY |
| | _SY | YYADSVKGRFTISRDNSKNTLYLQMNSLR | 28 | AKEGDYSRWAYDL |
| | | AEDTAVYYCAKEGDYSRWAYDLWGRGTLV | | |
| | | TVSS | | |
| 4 | FYIA | QVQLVESGGGVVQPGGSLRLSCAASGFTF | 29 | AASGFTESSYAMH |
| | germ | SSYAMHWVRQAPGKGLEWVAVISYDGSNK | 30 | VISYDGSNKY |
| | 2 | YYADSVKGRFTISRDNSKNTLYLQMNSLR | 31 | AKEGDSSRWSYDL |
| | | AEDTAVYYCAKEGDSSRWSYDLWGRGTLV | | |
| | | TVSS | | |
| 5 | FYIA | QVQLVESGGGVVQPGGSLRLSCAASGFTF | 32 | AASGFTFSSYDMH |
| | germ | SSYDMHWVRQAPGKGLEWVAVISYDGSNK | 33 | VISYDGSNKY |
| | 2_HC | YYADSVKGRFTISRDNSKNTLYLQMNSLR | 34 | AKEGDSSRWSYDL |
| | 1 | AEDTAVYYCAKEGDSSRWSYDLWGRGTLV | | |
| | | TVSS | | |
| 6 | FYIA | QVQLVESGGGVVQPGGSLRLSCAASGFAF | 35 | AASGFAFSSYDMH |
| | germ | SSYDMHWVRQAPGKGLEWVAVISYDGSNK | 36 | VISYDGSNKY |
| | 2_HC | YYADSVKGRFTISRDNSKNTLYLQMNSLR | 37 | AKEGDSSRWSYDL |
| | 2 | AEDTAVYYCAKEGDSSRWSYDLWGRGTLV | | |
| | | TVSS | | |
| 7 | FYIA | QVQLVESGGGVVQPGGSLRLSCAASGFTF | 38 | AASGFTFSSYAMH |
| | germ | SSYAMHWVRQAPGKGLEWVAVISYDGSNK | 39 | VISYDGSNKY |
| | 2_HC | YYADSVKGRFTISRDNSKNTLYLQMNSLR | 40 | AKEGDSSRWGYDL |
| | 3 | AEDTAVYYCAKEGDSSRWGYDLWGRGTLV | | |
| | | TVSS | | |
| 8 | FYIA | QVQLVESGGGVVQPGGSLRLSCAASGFTF | 41 | AASGFTESSYAMH |
| | germ | SSYAMHWVRQAPGKGLEWVAVVSYDGSNK | 42 | VVSYDGSNKY |
| | 2_HC | YYADSVKGRFTISRDNSKNTLYLQMNSLR | 43 | AKEGDSSRWSYDL |
| | 4 | AEDTAVYYCAKEGDSSRWSYDLWGRGTLV | | |
| | | TVSS | | |
| 9 | FYIA | QVQLVESGGGVVQPGGSLRLSCAASGFTF | 44 | AASGFTFSSYAMH |
| | germ | SSYAMHWVRQAPGKGLEWVAVVSYDGSNK | 45 | VVSYDGSNKY |
| | 2_HC | YYADSVKGRFTISRDNSKNTLYLQMNSLR | 46 | AKEGDSSRWGYDL |
| | 6 | AEDTAVYYCAKEGDSSRWGYDLWGRGTLV | | |
| | | TVSS | | |
| 10 | FYIA | QVQLVESGGGVVQPGGSLRLSCAASGFAF | 47 | AASGFAFSSYDMH |
| | germ | SSYDMHWVRQAPGKGLEWVAVVSYDGSNK | 48 | VVSYDGSNKY |
| | 2_HC7 | YYADSVKGRFTISRDNSKNTLYLQMNSLR | 49 | AKEGDSSRWGYDL |
| | | AEDTAVYYCAKEGDSSRWGYDLWGRGTLV | | |
| | | TVSS | | |
| 11 | FYIA | QVQLVESGGGVVQPGGSLRLSCAASGFTF | 50 | AASGFTFSSYDMH |
| | germ | SSYDMHWVRQAPGKGLEWVAVVSYDGSNK | 51 | VVSYDGSNKY |
| | 2_HC | YYADSVKGRFTISRDNSKNTLYLQMNSLR | 52 | AKEGDSSRWGYDL |
| | 8 | AEDTAVYYCAKEGDSSRWGYDLWGRGTLV | | |
| | | TVSS | | |

TABLE 2
Light chain variable sequences

| | | | CDR | |
|---|---|---|---|---|
| SEQ | | | SEQ | |
| ID | | | ID | CDR (1, 2, 3, |
| NO: | Name | VL sequence | NO: | respectively) |
| 12 | FYIA | QSALTQPASVSGSPGQSITISCTGTS | 53 | TGTSSDVGGYNYVS |
| | germ_ | SDVGGYNYVSWYQQHPGKAPKLMIYD | 54 | YDVTNRPS |
| | LC | VTNRPSGVSNRFSGSKSGNTASLTIS | 55 | SSYTIASTLVV |
| | | GLQAEDEADYYCSSYTIASTLVVEGG | | |
| | | GTKLTVL | | |
| 13 | FYIA | QSALTQPASVSGSPGQSITISCTG | 56 | TGTSSDVGGYNYVS |
| | germ_ | TSSDVGGYNYVSWYQQHPGKAPKL | 57 | YDVTNYPS |
| | SYLY_ | MIYDVTNYPSGVSNRFSGSKSGNT | 58 | SSYTVATTLVV |
| | LC | ASLTISGLQAEDEADYYCSSYTVA | | |
| | | TTLVVFGGGTKLTVL | | |
| 14 | FYIA | QSALTQPASVSGSPGQSITISCTGTS | 59 | TGTSSDVGGYNYVS |
| | germ_ | SDVGGYNYVSWYQQHPGKAPKLMIYD | 60 | YDVTNRPS |
| | LC2 | VTNRPSGVSNRFSGSKSGNTASLTIS | 61 | SSYTIASTPVV |
| | | GLQAEDEADYYCSSYTIASTPVVEGG | | |
| | | GTKLTVL | | |
| 15 | FYIA | QSALTQPASVSGSPGQSITISCTGTS | 62 | TGTSSDVGGYNYVS |
| | germ_ | SDVGGYNYVSWYQQHPGKAPKLMIYD | 63 | YDVTNRPS |
| | LC3 | VTNRPSGVSNRFSGSKSGNTASLTIS | 64 | SSYTIASTFVV |
| | | GLQAEDEADYYCSSYTIASTFVVEGG | | |
| | | GTKLTVL | | |
| 16 | FYIA | QSALTQPASVSGSPGQSITISCTGTS | 65 | TGTSSDVGGYNYVS |
| | germ_ | SDVGGYNYVSWYQQHPGKAPKLMIYD | 66 | YDVTNRPS |
| | LC4 | VTNRPSGVSNRFSGSKSGNTASLTIS | 67 | SSYAIASTLVV |
| | | GLQAEDEADYYCSSYAIASTLVVEGG | | |
| | | GTKLTVL | | |
| 17 | FYIA | QSALTQPASVSGSPGQSITISCTGTS | 68 | TGTSSDVGGYNYVS |
| | germ_ | SDVGGYNYVSWYQQHPGKAPKLMIYD | 69 | YDVTDRPS |
| | LC5 | VTDRPSGVSNRFSGSKSGNTASLTIS | 70 | SSYTIASTLVV |
| | | GLQAEDEADYYCSSYTIASTLVVEGG | | |
| | | GTKLTVL | | |
| 18 | FYIA | QSALTQPASVSGSPGQSITISCTGTS | 71 | TGTSSDVGGYNYVS |
| | germ_ | SDVGGYNYVSWYQQHPGKAPKLMIYD | 72 | YDVTDRPS |
| | LC6 | VTDRPSGVSNRFSGSKSGNTASLTIS | 73 | SSYAIASTLVV |
| | | GLQAEDEADYYCSSYAIASTLVVEGG | | |
| | | GTKLTVL | | |
| 19 | FYIA | QSALTQPASVSGSPGQSITISCTG | 74 | TGTSSDVGGYNYVS |
| | germ_ | TSSDVGGYNYVSWYQQHPGKAPKL | 75 | YDVTDRPS |
| | LC7 | MIYDVTDRPSGVSNRFSGSKSGNT | 76 | SSYAIASTPVV |
| | | ASLTISGLQAEDEADYYCSSYAIA | | |
| | | STPVVFGGGTKLTVL | | |

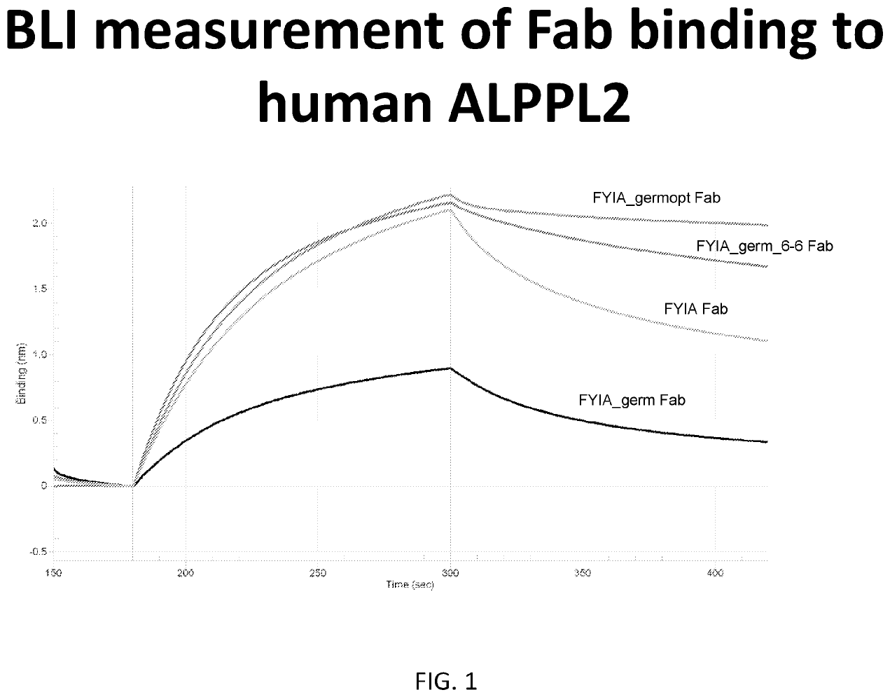

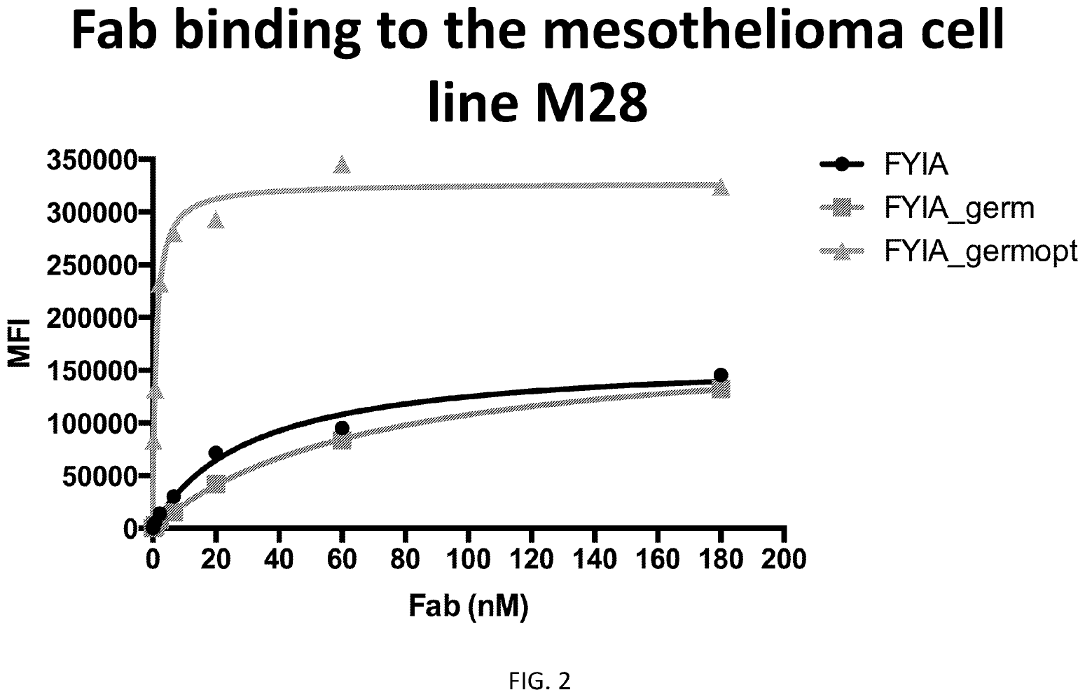

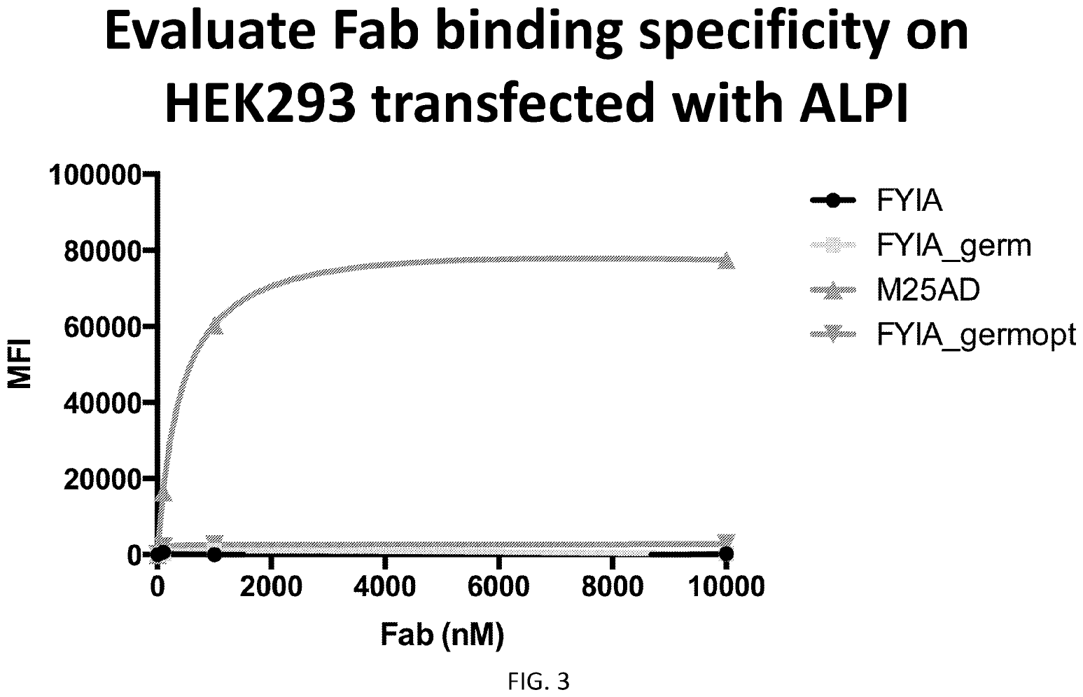

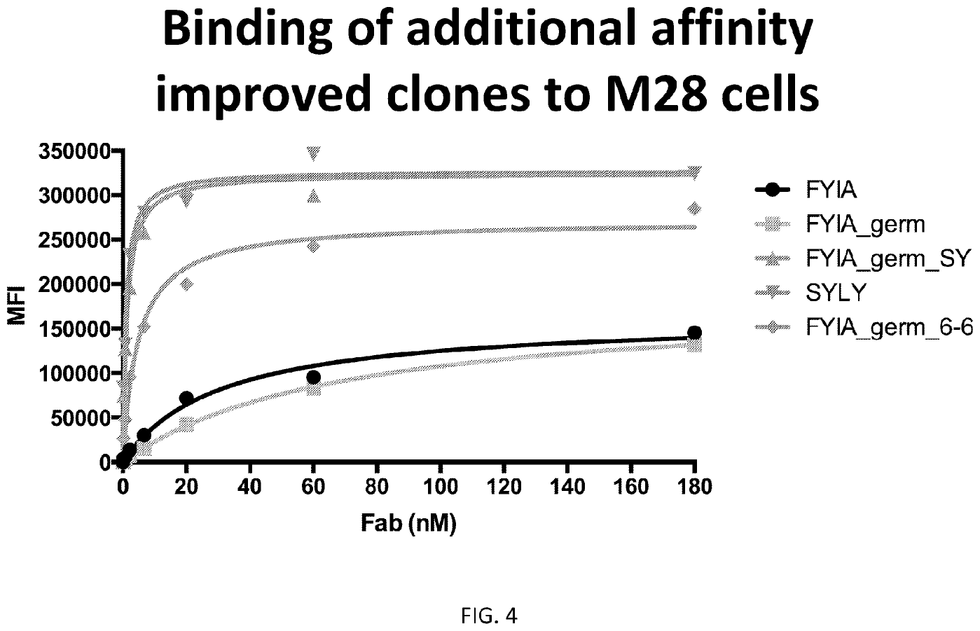

The following section describes the optimized properties of this new panel of FYIA variants including the lead antibody SYLY. With regard to improved binding affinity, in a side-by-side study of biolayer interferometry measurement of monovalent Fab binding to human ALPPL2, the calculated affinities are 8.6 nM for FYIA, 13.0 nM for FYIA_germ, 0.88 nM for FYIA_germ_6-6, and 0.19 nM for FYIA_germopt (aka SYLY) (FIG. 1). In a side-by-side study of monovalent Fab binding to mesothelioma cell line M28, the apparent binding affinities are 30.55 nM for FYIA, 69.41 nM for FYIA_germ, and 0.97 nM for FYIA_germopt (FIG. 2). To evaluate binding specificity, optimized FYIA variants were tested for binding to HEK293 transfected with human ALPI. As shown in FIG. 3, the optimized variants, like the parental FYIA, do not bind to ALPI. Additional clones (FYIA_germ_SY and FYIA_germ_6-6) beyond what is shown in FIG. 1 were also studied for binding to M28 cells as monovalent Fabs. As shown in FIG. 4, the apparent binding affinities are 1.27 nM for FYIA_germ_SY and 4.82 nM for FYIA_germ_6-6. Table 3 summarizes results of the cell binding study. In the monovalent Fab form, the lead antibody FYIA_germopt (aka SYLY) has over 30-fold improvement over the parental FYIA.

TABLE 3

| Fab | Apparent KD |
|---|---|
| FYIA | 30.55 |
| FYIA_germ | 69.41 |
| FYIA_germ_SY | 1.271 |
| SYLY (FYIA_germopt) | 0.9668 |
| FYIA_germ_6-6 | 4.823 |

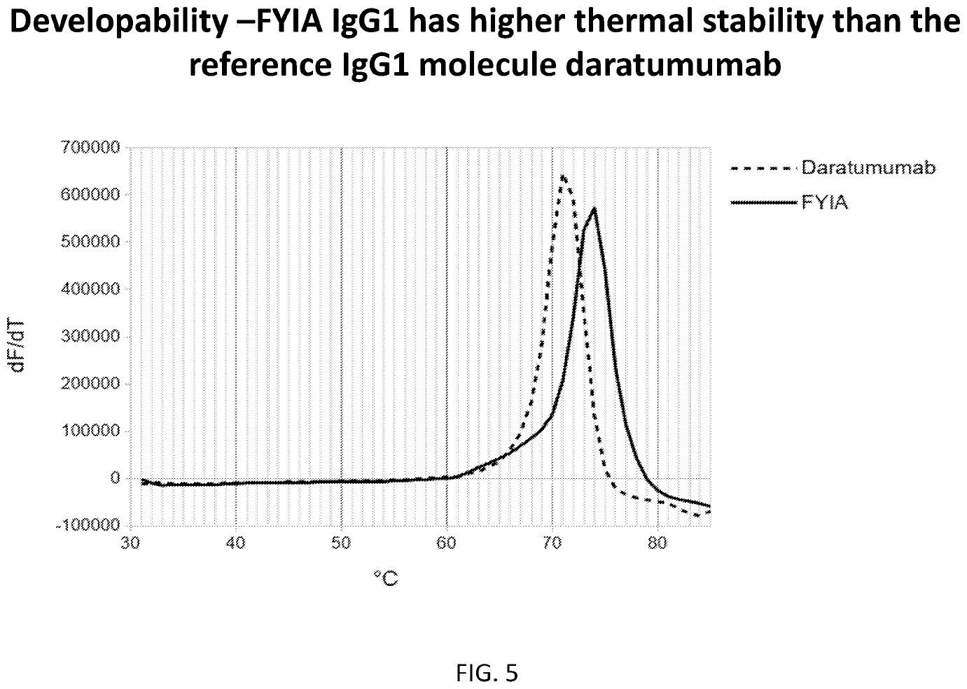

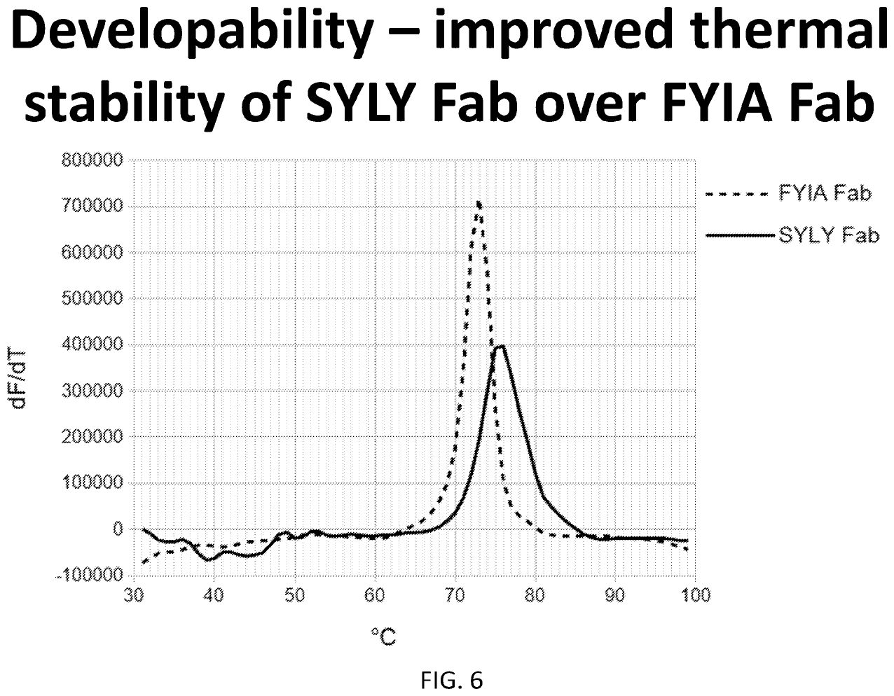

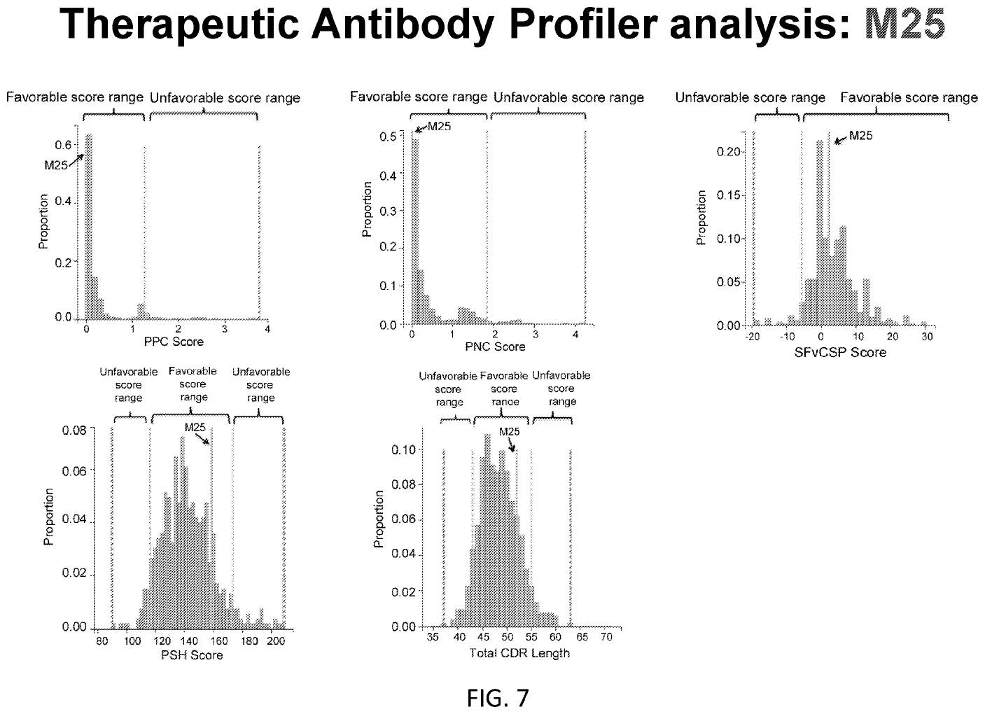

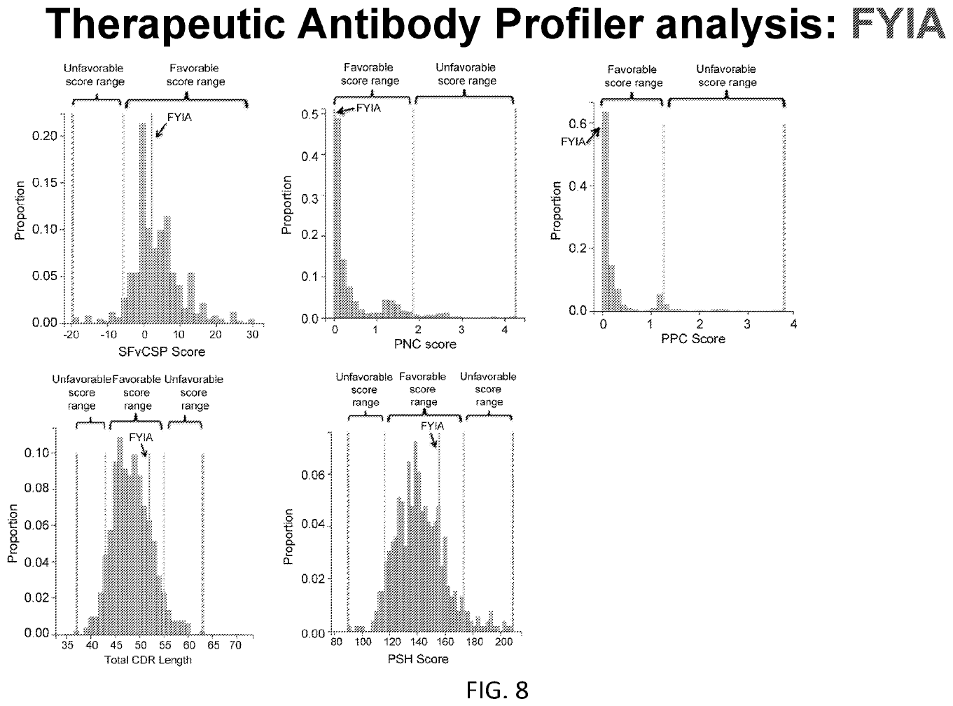

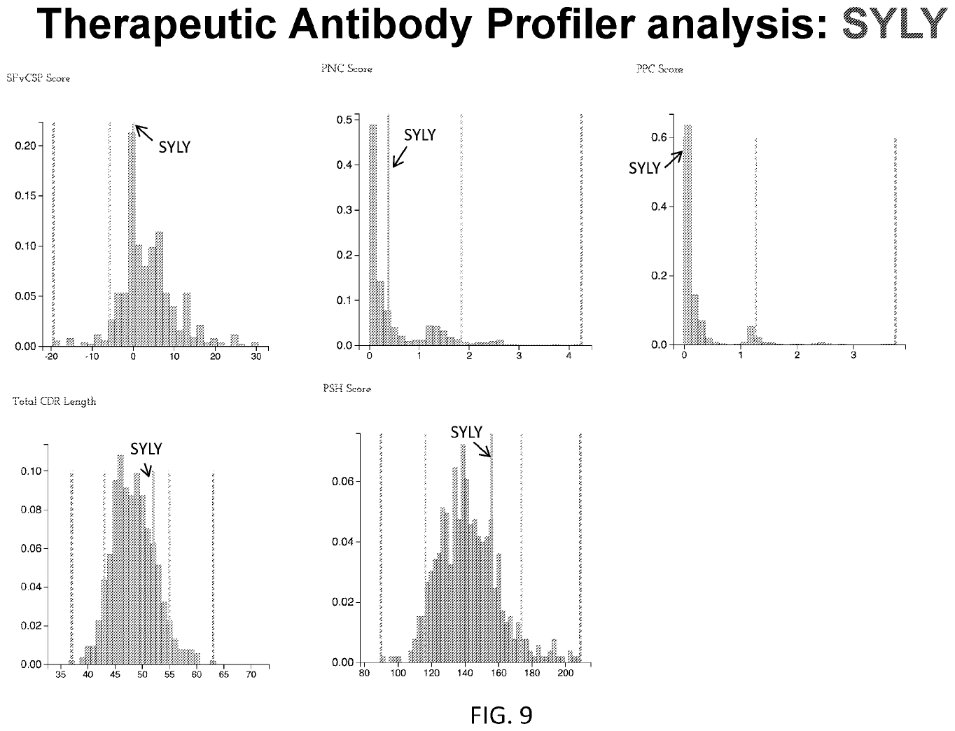

With regard to developability, in a side-by-side study of thermal-induced aggregation assay, SYLY showed improved thermal stability with higher aggregation temperature (Tagg) than FYIA (76° C. vs. 73° C., FIG. 5). For reference, the parental FYIA (as a human IgG1 molecule) and the clinically used daratumumab (in IgG1 form) were also studied side-by-side. As shown in FIG. 6, FYIA showed higher thermal stability than daratumumab. In a second set of analysis where the variable domain sequences of the M25/FYIA/SYLY series were analyzed against five developability guidelines derived from clinical-stage therapeutic values (Raybould, M. I. J. et al, 2019), the parental M25 and its derivatives (FYIA and SYLY) all showed favorable property with no potential developability issues identified (FIG. 7 for M25, FIG. 8 for FYIA and FIG. 9 for SYLY).

We then used SYLY to construct an ALPPL2×CD3 bispecific T cell engager. We humanized the SP34 murine anti-human CD3 antibody (Table 4).

TABLE 4
CD3-binding sequences

| | | | | |
|---|---|---|---|---|
| 77 | VH of | EVQLVESGGGLVQPGGSLRLSCAA | 79 | ASGFTFNTYAMN |
| | hSP34k2 | SGFTENTYAMNWVRQAPGKGLEWV | 80 | RIRSKYNN |
| | | GRIRSKYNNYATYYADSVKDRFTI | 81 | VRHGNFGNSYVSWFAY |
| | | SRDDSKNSLYLQMNSLKTEDTAVY | | |
| | | YCVRHGNFGNSYVSWFAYWGQGTL | | |
| | | VTVSS | | |
| 78 | VL of | DIQMTQSPSSLSASVGDRVTITCR | 82 | SSTGAVTTSNYAN |
| | hSP34k2 | SSTGAVTTSNYANWVQQKPGKAPK | 83 | GGTNKRAP |
| | | GLIGGTNKRAPGVPSRFSGSLIGD | 84 | ALWYSNLWV |
| | | KATLTISSLQPEDFATYYCALWYS | | |
| | | NLWVFGQGTKVEIK | | |

There are over 80 different ways to construct a bispecific antibody (FIGS. 10A and 10B, adopted from Brinkmann et al), which can be used to construct the ALPPL2×CD3. In addition, we developed a bispecific using a different molecular architecture. We constructed a dually stabilized diabody (DSDbody) using an inter-chain disulfide link and CH1/CL pairing. The CH1/CL pairing also forces the heterodimer formation. The DSDbody sequences are shown in Table 5.

TABLE 5

| | | |
|---|---|---|
| 85 | ALPPL2 × CD3 DSDbody: | EVQLVESGGGLVQPGGSLRLSCAASGFT |
| | Chain 1: antiCD3VH-TAC- | FNTYAMNWVRQAPGKGLEWVGRIRSKYN |
| | G4S-FYIA_germoptVL-CH1 | NYATYYADSVKDRFTISRDDSKNSLYLQ |
| | (CH1 underlined) | MNSLTACDTAVYYCVRHGNFGNSYVSWF |
| | | AYWGQGTLVTVSSGGGGSQSALTQPASV |
| | | SGSPGQSITISCTGTSSDVGGYNYVSWY |
| | | QQHPGKAPKLMIYDVTYRPSGVSNRFSG |
| | | SKSGNTASLTISGLQAEDEADYYCSSYT |
| | | VATTLVVFGGGTKLTVLASTKGPSVFPL |
| | | APSSKSTSGGTAALGCLVKDYFPEPVTV |
| | | SWNSGALTSGVHTFPAVLQSSGLYSLSS |
| | | VVTVPSSSLGTQTYICNVNHKPSNTKVD |
| | | KKVEPKSC |
| 86 | ALPPL2 × CD3 DSDbody : | EVQLVESGGGLVQPGGSLRLSCAASGFT |
| | Chain 2: FYIA_germoptVH- | FSAYAMHWVRQAPGKGLEWVAVISYEGY |
| | TAC-G4S-antiCD3VL-CL (CL | NAYYADSVKGRFTISRDNSKNTLYLQMN |
| | underlined) | SLTACDTAVYYCAKEGDYSRWAYDYWGR |
| | | GTLVTVSSGGGGSDIQMTQSPSSLSASV |
| | | GDRVTITCRSSTGAVTTSNYANWVQQKP |
| | | GKAPKGLIGGTNKRAPGVPSRESGSLIG |
| | | DKATLTISSLQPEDFATYYCALWYSNLW |
| | | VFGQGTKVEIKRTVAAPSVFIFPPSDEQ |
| | | LKSGTASVVCLLNNFYPREAKVQWKVDN |
| | | ALQSGNSQESVTEQDSKDSTYSLSSTLT |
| | | LSKADYEKHKVYACEVTHQGLSSPVTKS |
| | | FNRGEC |
| 87 | SYLY-based diabody-CH1/CL | EVQLVESGGGLVQPGGSLRLSCAASGFT |
| | (without disulfide | FNTYAMNWVRQAPGKGLEWVGRIRSKYN |
| | stabilization): | NYATYYADSVKDRFTISRDDSKNSLYLQ |
| | Chain 1: antiCD3VH-G4S- | MNSLKTEDTAVYYCVRHGNFGNSYVSWF |
| | FYIA_germoptVL-CH1 (CH1 | AYWGQGTLVTVSSGGGGSQSALTQPASV |
| | underlined) | SGSPGQSITISCTGTSSDVGGYNYVSWY |
| | | QQHPGKAPKLMIYDVTYRPSGVSNRFSG |
| | | SKSGNTASLTISGLQAEDEADYYCSSYT |
| | | VATTLVVFGGGTKLTVLASTKGPSVFPL |
| | | APSSKSTSGGTAALGCLVKDYFPEPVTV |
| | | SWNSGALTSGVHTFPAVLQSSGLYSLSS |
| | | VVTVPSSSLGTQTYICNVNHKPSNTKVD |
| | | KKVEPKSC |
| 88 | SYLY-based diabody-CH1/CL | MGWSCIILFLVATATGVHSEVQLVESGG |
| | (without disulfide | GLVQPGGSLRLSCAASGFTFSAYAMHWV |
| | stabilization): | RQAPGKGLEWVAVISYEGYNAYYADSVK |
| | Chain 2: FYIA_germoptVH- | GRFTISRDNSKNTLYLQMNSLRAEDTAV |
| | G4S-antiCD3VL-CL (CL | YYCAKEGDYSRWAYDYWGRGTLVTVSSG |
| | underlined) | GGGSDIQMTQSPSSLSASVGDRVTITCR |
| | | SSTGAVTTSNYANWVQQKPGKAPKGLIG |
| | | GTNKRAPGVPSRESGSLIGDKATLTISS |
| | | LQPEDFATYYCALWYSNLWVFGQGTKVE |
| | | IKRTVAAPSVFIFPPSDEQLKSGTASVV |
| | | CLLNNFYPREAKVQWKVDNALQSGNSQE |
| | | SVTEQDSKDSTYSLSSTLTLSKADYEKH |
| | | KVYACEVTHQGLSSPVTKSENRGEC |

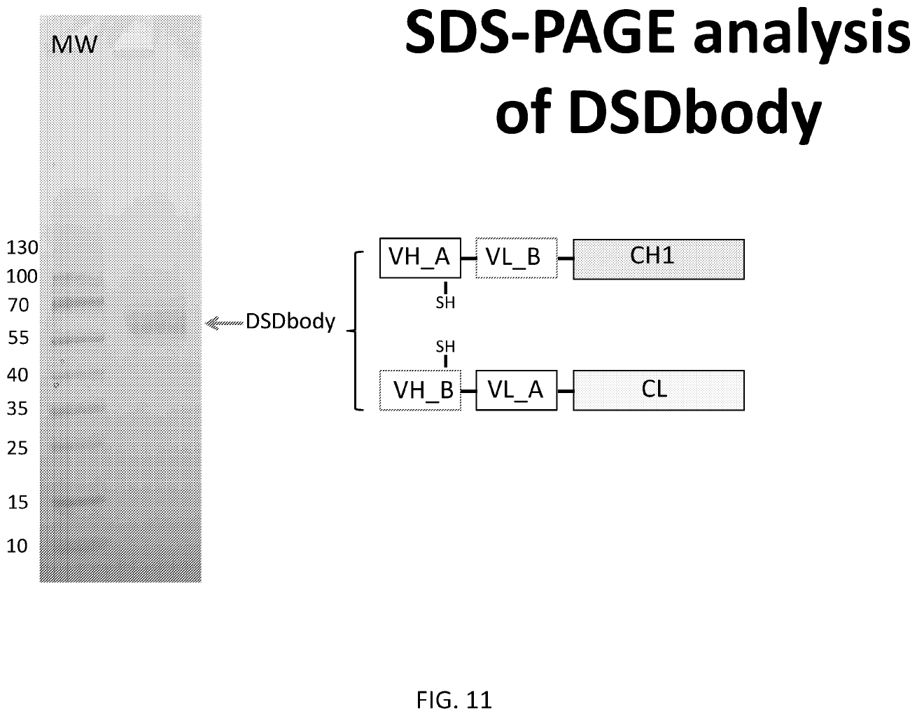

A hexahistidine tag (SEQ ID NO: 102) was added to the C terminal of CH1 for purification by metal affinity chromatography using nickel-charged affinity resin (Ni-NTA agarose). DSDbody was produced in HEK293A or ExpiCHO cells by transient transfection, purified by NI-NTA and analyzed by reducing SDS-PAGE (FIG. 11). The bispecific can also be constructed from a variant of DSDbody without the interchain disulfide bond (sequences shown in Table 6).

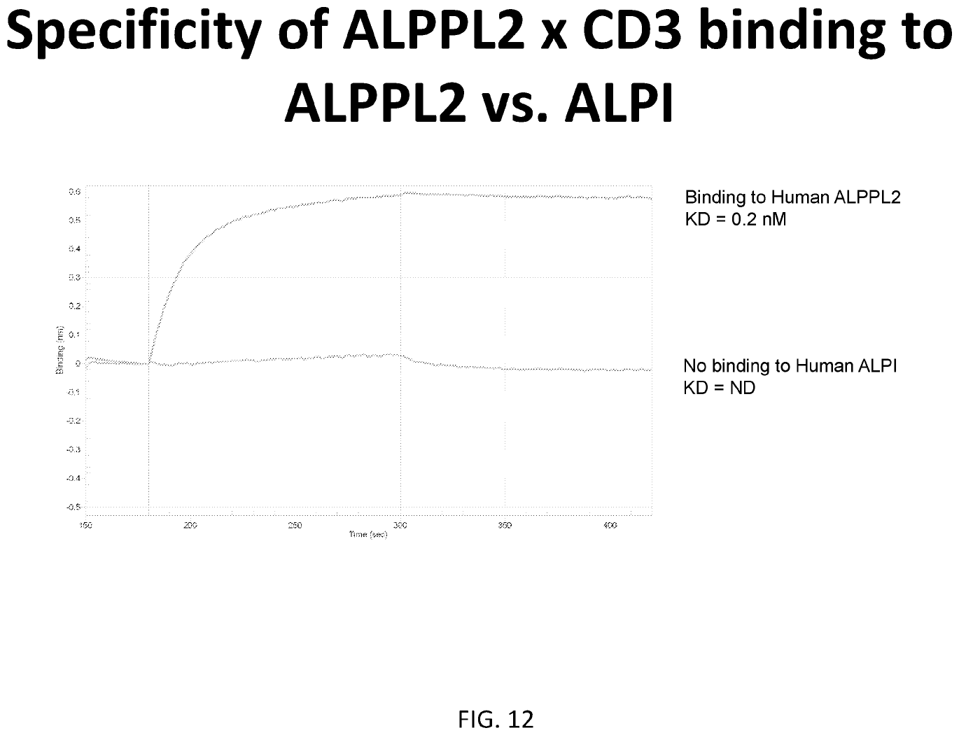

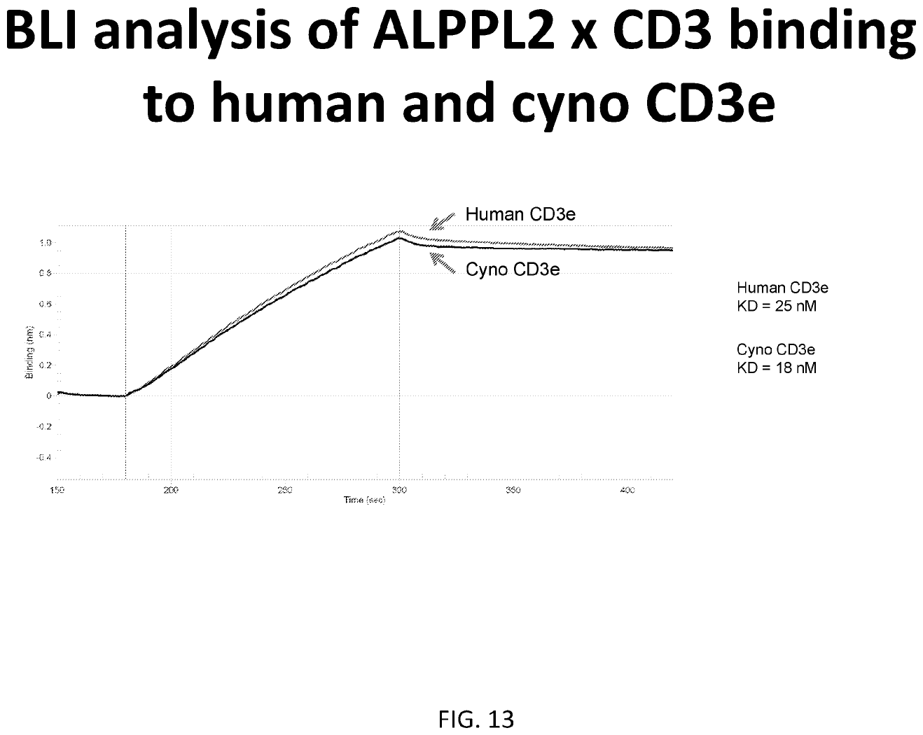

Biolayer interferometry analysis showed that the SYLY-based bispecific ALPPL2×CD3 DSDbody binds to human ALPPL2 with high affinity (KD˜0.2 nM) and specificity (no binding to human ALPI) (FIG. 12). The DSDbody was also evaluated by biolayer interferometry for cross-species binding to both human and cynomolgus monkey CD3 epsilon chains. As shown in FIG. 13, the SYLY-based DSDbody showed similar binding affinities to human and cynomolgus monkey CD3 molecules (20 nM and 18 nM, respectively).

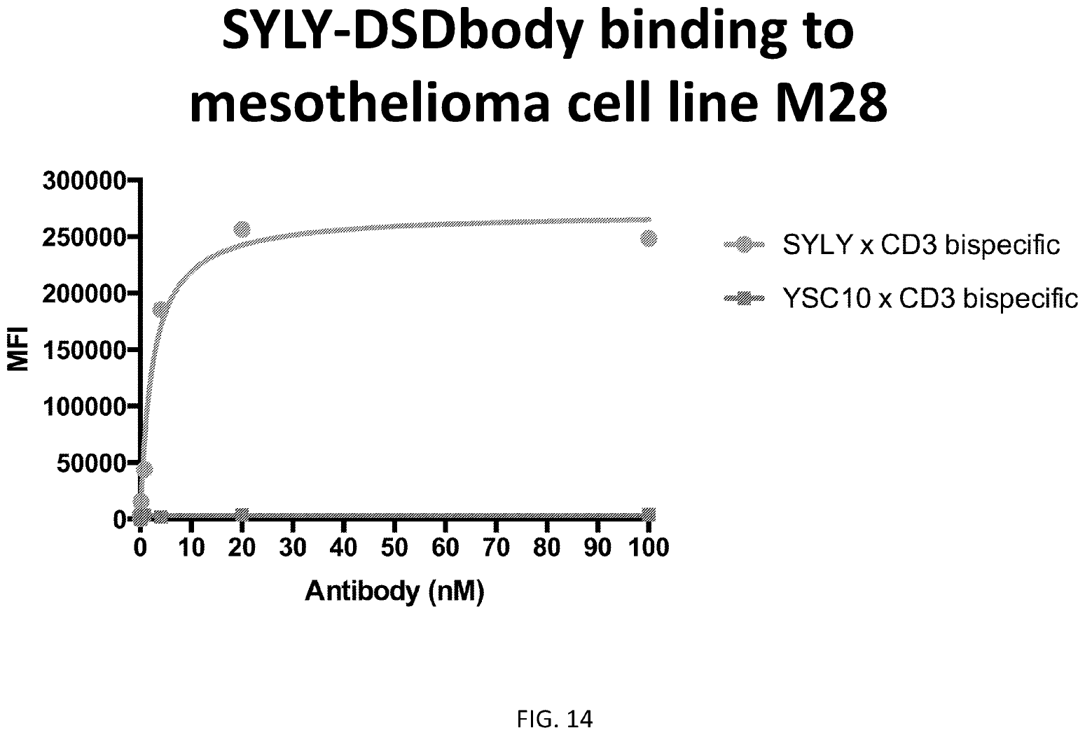

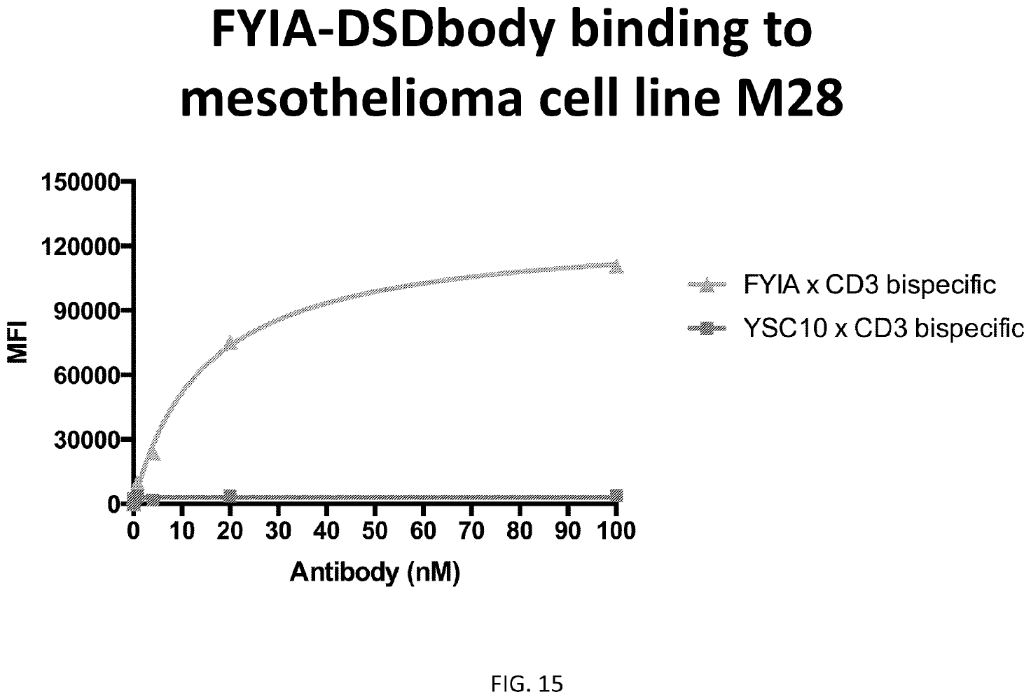

Binding of the SYLY-based bispecific ALPPL2×CD3 DSDbody to tumor cells were studied by flow cytometry using the mesothelioma cell line M28. As shown in FIG. 14, the DSDbody binds to M28 cells with an apparent binding affinity of 2.4 nM. No binding was detected for a control DSDbody that is constructed from a non-binding human antibody YSC10 and the same anti-CD3 antibody. For comparison, a reference DSDbody constructed from the parental FYIA was also studied on M28 cells using flow cytometry. As shown in FIG. 15, the FYIA-based DSDbody showed an apparent binding affinity of 14.8 nM to M28 cells.

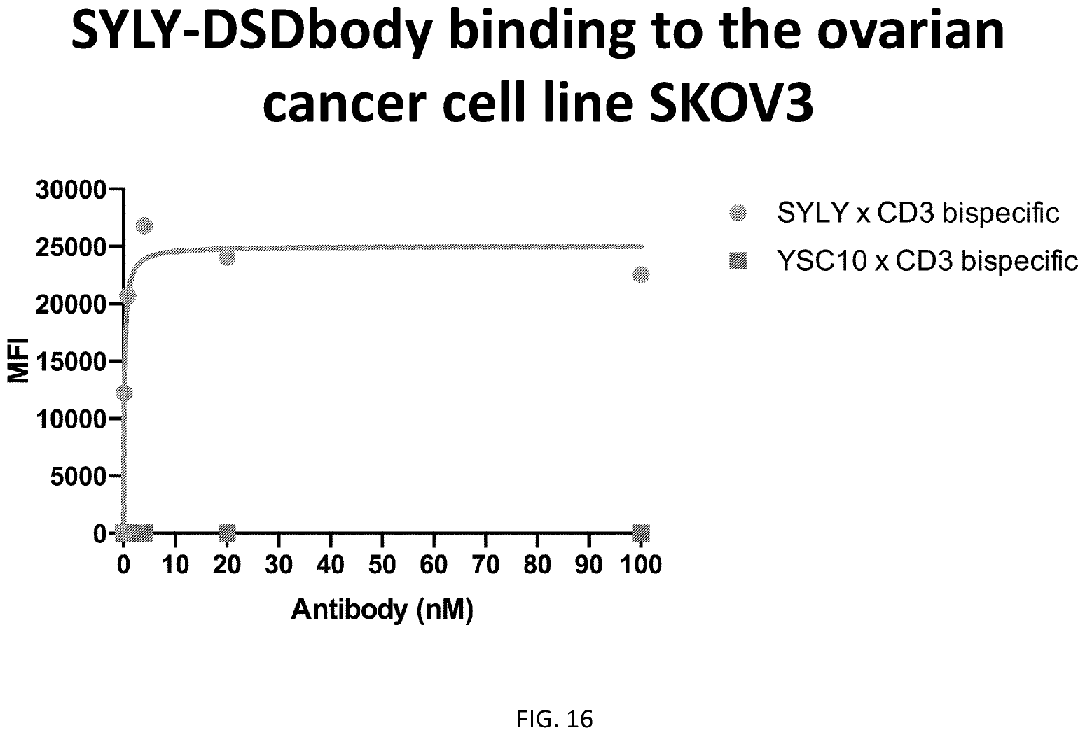

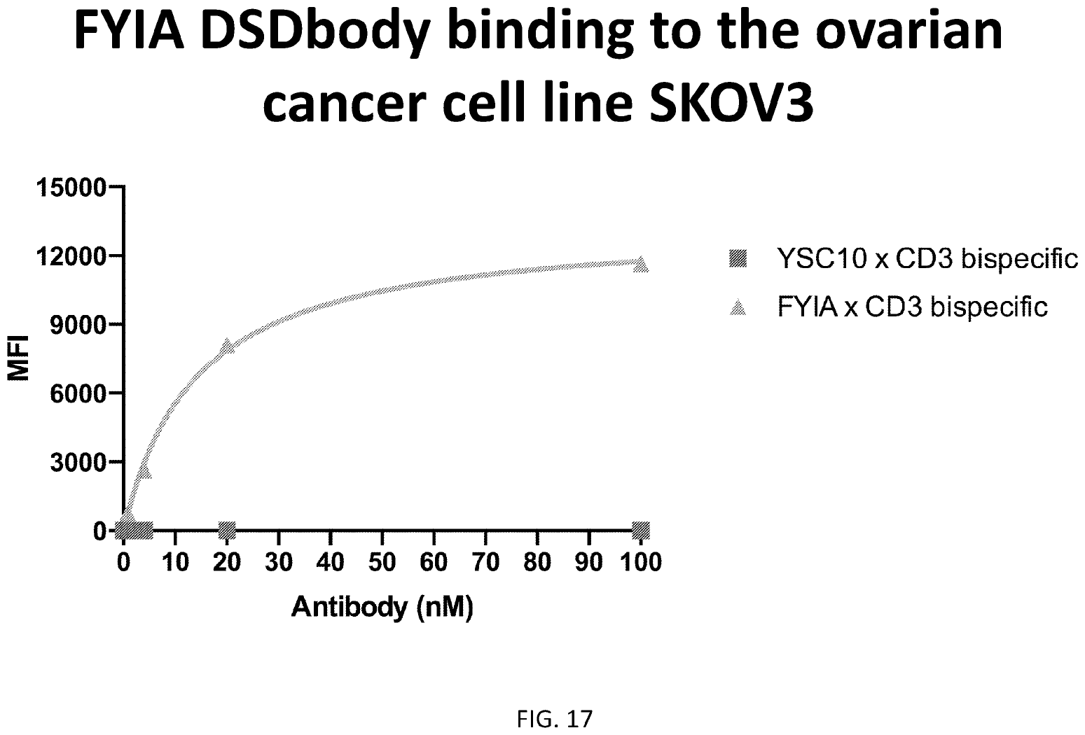

In addition to mesothelioma cells, binding of the SYLY-based bispecific ALPPL2×CD3 DSDbody was also studied on the ovarian cancer cell line SKOV3. As shown in FIG. 16, the SYLY-based ALPPL2×CD3 bispecific DSDbody binds to SKOV3 cells with an apparent affinity of 0.19 nM. No binding was detected by the control bispecific YSC10×CD3 that is built on a non-binding isotype control antibody YSC10. For comparison, a reference DSDbody constructed from the parental FYIA was also studied on SKOV3 cells using flow cytometry. As shown in FIG. 17, the FYIA-based DSDbody showed an apparent binding affinity of 14.2 nM to SKOV3 cells.

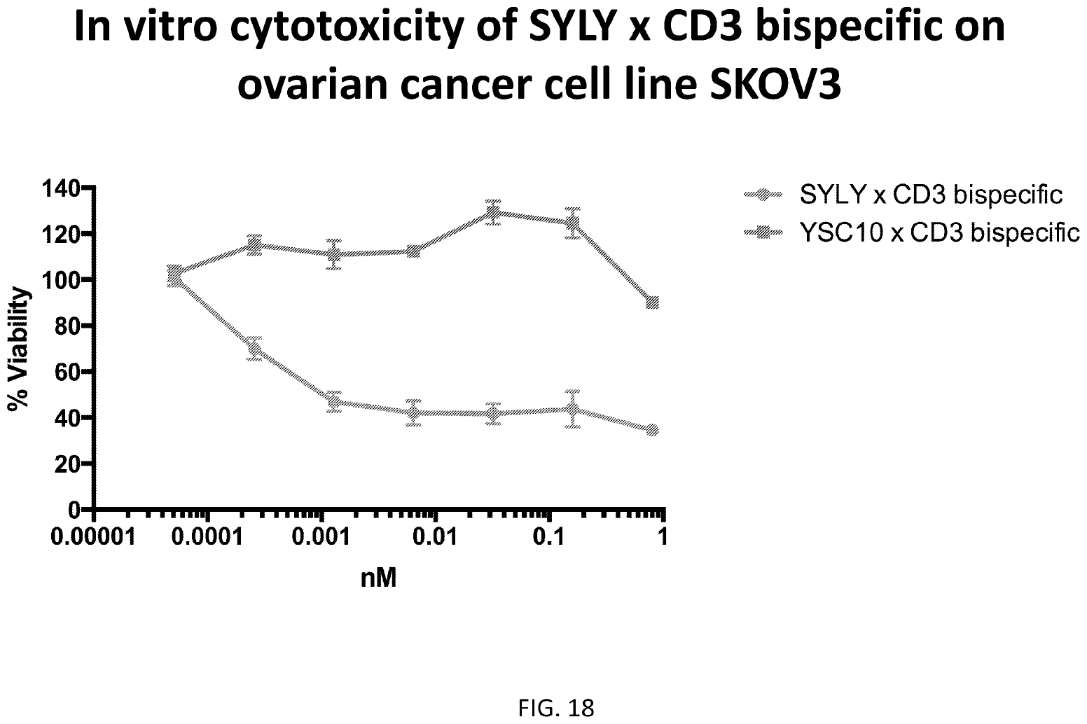

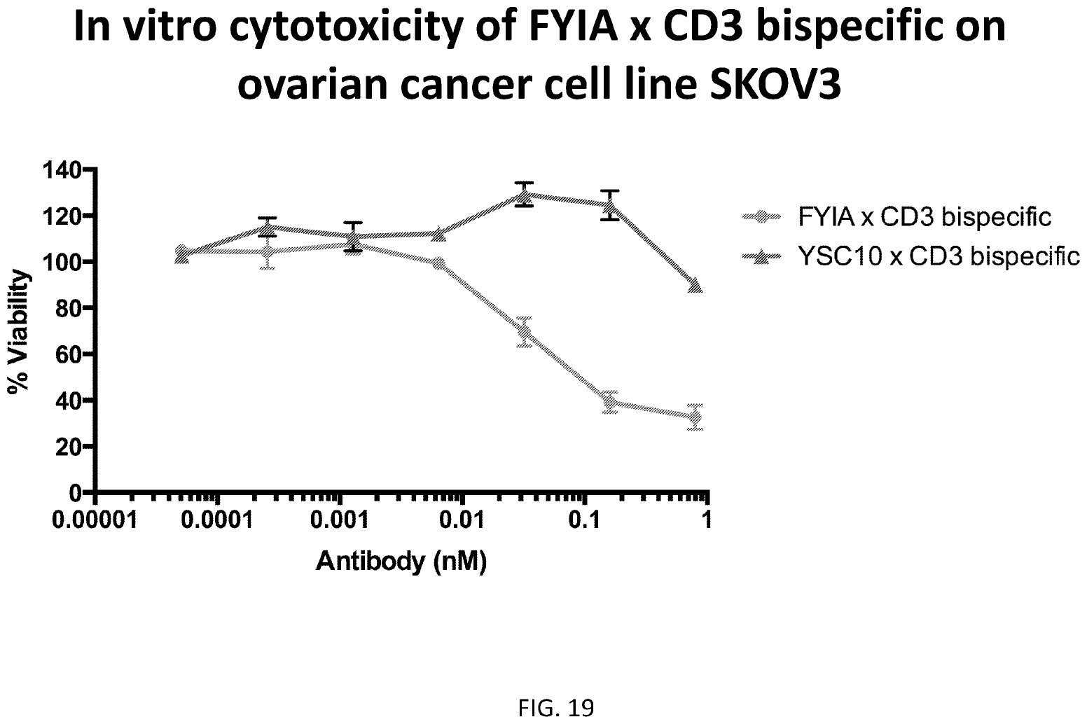

Target-dependent cytotoxicity was studied using ALPPL2 expressing tumor cells with human PBMCs as a source of effector cells. The SYLY-based bispecific DSDbody was incubated with the ovarian cancer cell line SKOV3 (target) in the presence of human PBMCs (effector) at E:T ratio=10:1 for 96h. Cell viability was measured using the Calcein Am solution. As shown in FIG. 18, the SYLY-based DSDbody kills SKOV3 cells with a calculated EC50 of 0.3 pM. No killing was detected by the control bispecific YSC10×CD3 DSDbody that is built on a non-binding isotype control antibody YSC10. For comparison, the reference DSDbody constructed from the parental FYIA was also studied on SKOV3 cells under the same condition and E:T ratio. As shown in FIG. 19, the calculated EC50 for the FYIA DSDbody is 62.8 pM.

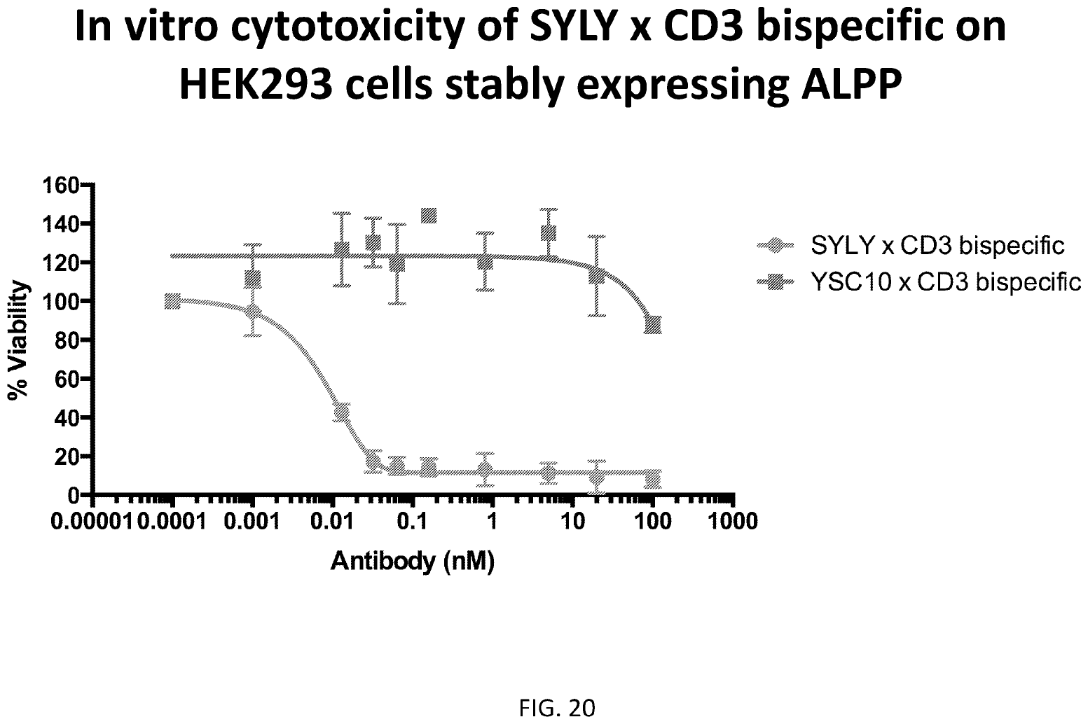

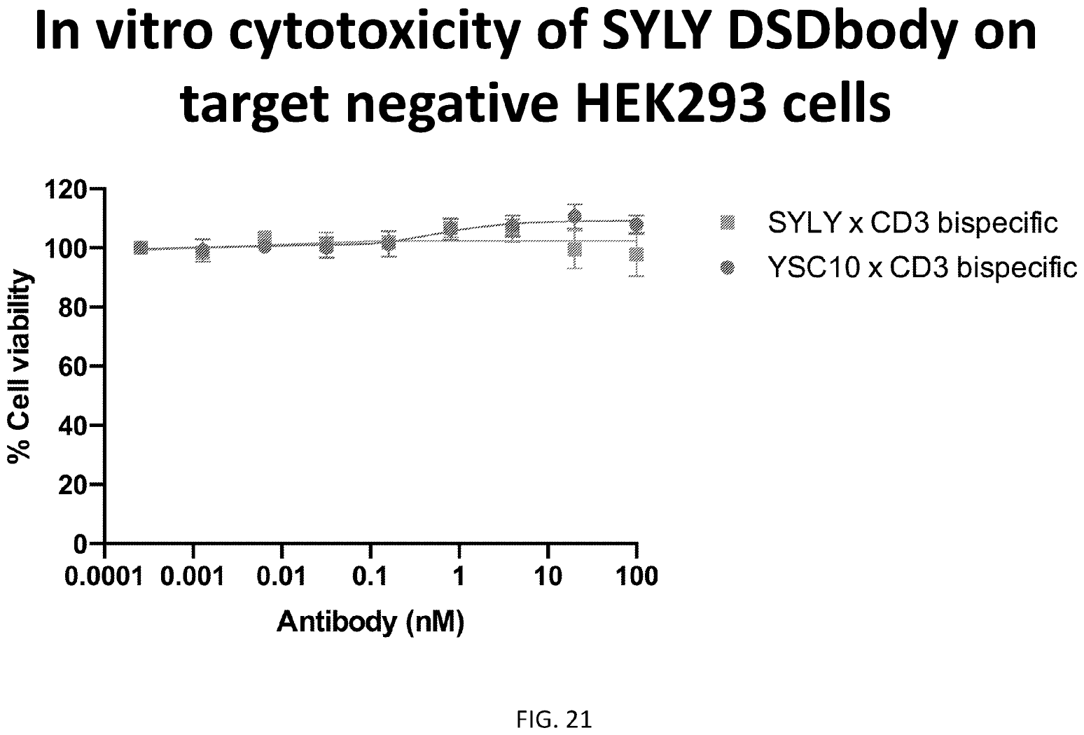

The target-dependent cytotoxicity was further studied using HEK293 cells stably expressing ALPP (target). The SYLY-based DSDbody was incubated with HEK293-ALPP cells in the presence of human PBMC at E:T ratio=10:1 for 72h, followed by cell viability assessment by Calcein AM. As shown in FIG. 20, the calculated EC50 for the SYLY DSDbody is 8.5 pM, and >100 nM for the control bispecific YSC10×CD3 DSDbody. No killing was observed on HEK293 cells that do not express the target antigen (ALPP or ALPPL2, FIG. 21).

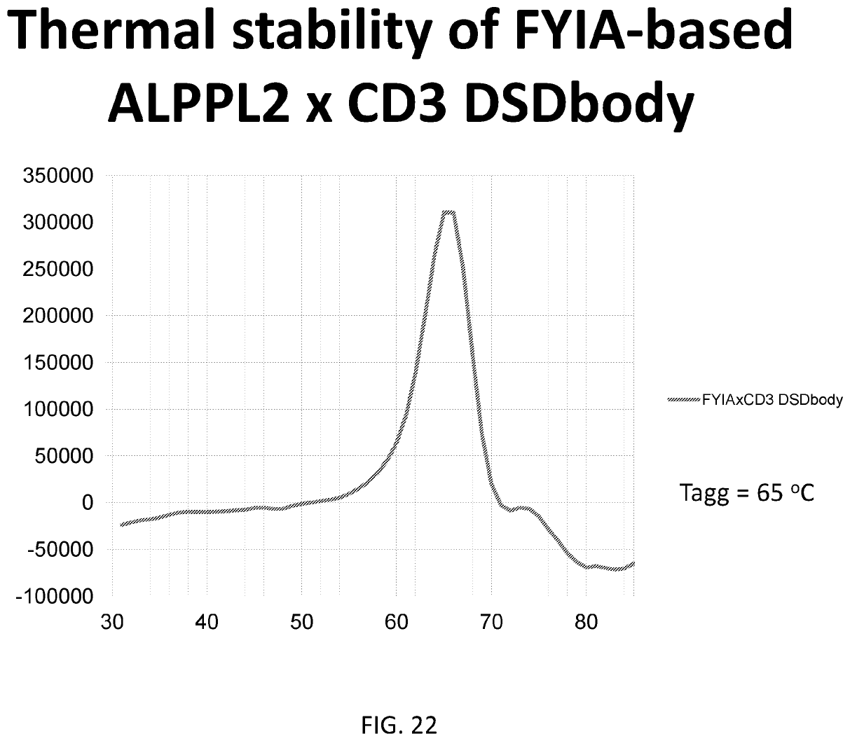

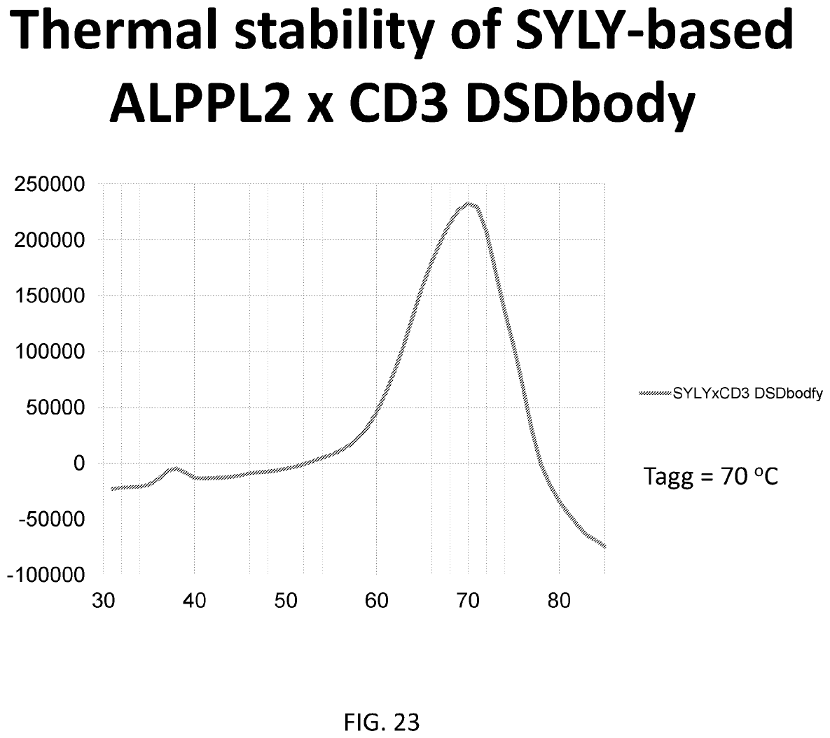

The DSDbody showed excellent thermal stability. In the thermal induced aggregation assay, the FYIA-based ALPP12×CD3 DSDbody showed an aggregation temperature (Tagg) of 65° C. (FIG. 22), and the SYLY-based ALPPL2×CD3 DSDbody showed an even higher Tagg of 70° C. (FIG. 23).

### Example 2

This example shows the effect of antibodies described herein on a reporter-expressing pancreatic cancer line, AsPC1.

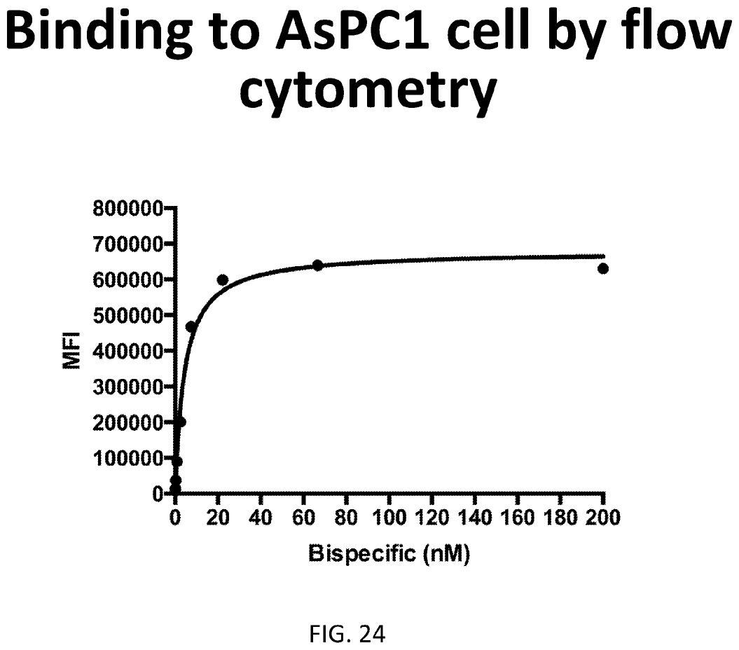

The bispecific (ALPPL2×CD3) SYLY DSDBody was incubated with AsPC1 cells at room temperature for 1 h, washed and further incubated with anti-tag (hexahistidine (SEQ ID NO: 102)) antibodies for detection by flow cytometry. MFI values were curve-fitted with the K_{D} estimated as 4.35±0.68 nM. See, FIG. 24.

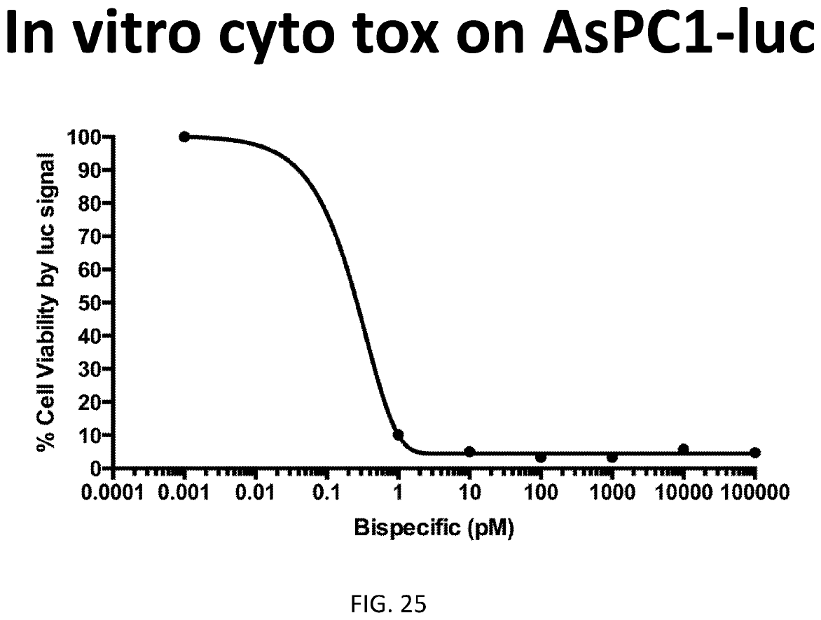

An in vitro cyto tox assay using a reporter expressing cancer cell line was performed as follows. Firefly luciferase expressing AsPC1-luc cells were plated into 96-well tissue culture plate at 3,000 cell/well and cultured overnight. 30,000 cell/well of PBMC were added to target cells (E:T ratio=10:1). Antibodies were serially diluted and incubated for 72 hours. Cell viability was measured by bioluminescence using a microtiter plate reader. The percent of cell viability was derived from normalized luciferase signal, and curve-fitted to generate an estimated EC50 (less than 1 pM, about 0.245 pM). See, FIG. 25.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An antibody comprising a variable region that specifically binds to ALPPL2 and ALPP, wherein the antibody comprises a heavy chain variable region and a light chain variable region,
wherein complementarity determining region (CDR) 1, CDR2 and CDR3 of the heavy chain variable region are selected from the following sets:
SEQ ID NOs: 20, 21, and 22;
SEQ ID NOs: 23, 24, and 25;
SEQ ID NOs: 26, 27, and 28;
SEQ ID NOs: 29, 30, and 31;
SEQ ID NOs: 32, 33, and 34;
SEQ ID NOs: 35, 36, and 37;
SEQ ID NOs: 38, 39, and 40;
SEQ ID NOs: 41, 42, and 43;
SEQ ID NOs: 44, 45, and 46;
SEQ ID NOs: 47, 48, and 49; or
SEQ ID NOs: 50, 51, and 52;
CDR1, CDR2, and CDR3 of the light chain variable region are selected from the following sets:
SEQ ID NOs: 53, 54, and 55;
SEQ ID NOs: 56, 57, and 58;
SEQ ID NOs: 59, 60, and 61;
SEQ ID NOs: 62, 63, and 64;
SEQ ID NOs: 65, 66, and 67;
SEQ ID NOs: 68, 69, and 70;
SEQ ID NOs: 71, 72, and 73; or
SEQ ID NOs: 74, 75, and 76,
with the proviso that the antibody does not have the heavy chain variable region of M25FYIA and the light chain variable region of M25FYIA.

2. The antibody of claim 1, wherein
CDR1, CDR2, and CDR3 of the heavy chain variable region comprise SEQ ID NO: 23, SEQ ID NO: 24, and SEQ ID NO: 25, respectively; and
CDR1, CDR2, and CDR3 of the light chain variable region comprise SEQ ID NO: 56, SEQ ID NO: 57, and SEQ ID NO: 58, respectively; or
CDR1, CDR2, and CDR3 of the heavy chain variable region comprise SEQ ID NO: 26, SEQ ID NO: 27, and SEQ ID NO: 28, respectively; and
CDR1, CDR2, and CDR3 of the light chain variable region comprise SEQ ID NO: 56, SEQ ID NO: 57, and SEQ ID NO: 58, respectively; or
CDR1, CDR2, and CDR3 of the heavy chain variable region comprise SEQ ID NO: 44, SEQ ID NO: 45, and SEQ ID NO: 46, respectively; and
CDR1, CDR2, and CDR3 of the light chain variable region comprise SEQ ID NO: 71, SEQ ID NO: 72, and SEQ ID NO: 73, respectively; or
CDR1, CDR2, and CDR3 of the heavy chain variable region comprise SEQ ID NO: 38, SEQ ID NO: 39, and SEQ ID NO: 40, respectively; and
CDR1, CDR2, and CDR3 of the light chain variable region comprise SEQ ID NO: 65, SEQ ID NO: 66, and SEQ ID NO: 67, respectively; or
CDR1, CDR2, and CDR3 of the heavy chain variable region comprise SEQ ID NO: 41, SEQ ID NO: 42, and SEQ ID NO: 43, respectively; and
CDR1, CDR2, and CDR3 of the light chain variable region comprise SEQ ID NO: 53, SEQ ID NO: 54, and SEQ ID NO: 55, respectively; or
CDR1, CDR2, and CDR3 of the heavy chain variable region comprise SEQ ID NO: 35, SEQ ID NO: 36, and SEQ ID NO: 37, respectively; and
CDR1, CDR2, and CDR3 of the light chain variable region comprise SEQ ID NO: 59, SEQ ID NO: 60, and SEQ ID NO: 61, respectively.

3. The antibody of any one of claim 1-2, wherein
the heavy chain variable region is selected from the group consisting of SEQ ID NOs: 1, 2, 3, 4, 5, 6, 7, 8, 9, 10 and 11.

4. The antibody of any one of claim 1-3, wherein
the light chain variable region is selected from the group consisting of SEQ ID NOs: 12, 13, 14, 15, 16, 17, 18, and 19.

5. The antibody of claim 1, wherein
the heavy chain variable region comprises SEQ ID NO: 1; and
the light chain variable region comprises SEQ ID NO: 12; or
the heavy chain variable region comprises SEQ ID NO: 2; and
the light chain variable region comprises SEQ ID NO: 13; or
the heavy chain variable region comprises SEQ ID NO: 3; and
the light chain variable region comprises SEQ ID NO: 13; or
the heavy chain variable region comprises SEQ ID NO: 9; and
the light chain variable region comprises SEQ ID NO: 18; or
the heavy chain variable region comprises SEQ ID NO: 1; and
the light chain variable region comprises SEQ ID NO: 12; or
the heavy chain variable region comprises SEQ ID NO: 7; and
the light chain variable region comprises SEQ ID NO: 16; or
the heavy chain variable region comprises SEQ ID NO: 8; and
the light chain variable region comprises SEQ ID NO: 12; or
the heavy chain variable region comprises SEQ ID NO: 6; and
the light chain variable region comprises SEQ ID NO: 14.

6. The antibody of any one of claims 1-5, wherein the antibody is an IgG, IgA or IgE antibody.

7. The antibody of claim 6, wherein the antibody is an IgG antibody.

8. The antibody of claim 7, wherein the antibody is an IgG1, IgG2, IgG3, or IgG4 antibody.

9. The antibody of any one of claims 1-8, wherein the antibody is a monospecific antibody.

10. The antibody of claim 9, wherein the antibody is linked to a cytotoxic agent.

11. The antibody of claim 10, wherein the cytotoxic agent is a radionucleotide.

12. The antibody of any one of claims 1-8, wherein the antibody is a bi-specific antibody comprising a second variable region that specifically binds to a second target protein, wherein the antibody comprises a second heavy chain variable region and a second light chain variable region.

13. The antibody of claim 12, wherein the second target protein is expressed on the surface of a human immune effector cell.

14. The antibody of claim 12, wherein the second target protein is human CD3.

15. The antibody of claim 14, wherein the CDR1, CDR2, and CDR3 of the second heavy chain variable region comprise SEQ ID NO: 79, SEQ ID NO: 80 and SEQ ID NO: 81 and the CDR1, CDR2, and CDR3 of the second light chain variable region comprise SEQ ID NO: 82, SEQ ID NO: 83 and SEQ ID NO: 84.

16. The antibody of claim 14, wherein the second heavy chain variable region comprises SEQ ID NO: 77 and the second light chain variable region comprises SEQ ID NO:78.

17. The antibody of claim 14, comprising SEQ ID NO:85 and SEQ ID NO:86.

18. The antibody of claim 14, comprising SEQ ID NO:87 and SEQ ID NO:88.

19. A pharmaceutical composition comprising the antibody of any one of claims 1-18.

20. A nucleic acid encoding the antibody of any one of claims 1-18.

21. A vector comprising the nucleic acid sequence of claim 20.

22. A cell comprising the nucleic acid of claim 20 or the vector of claim 21.

23. The cell of claim 22, wherein the cell is a mammalian cell.

24. A method for producing an antibody, the method comprising culturing the cell of any one of claim 22 or 23 under conditions to allow for production of the antibody.

25. A method of killing a cancer cell, the method comprising, contacting the antibody of any one of claims 1-18 to a cancer cell.

26. The method of claim 25, wherein the antibody is a bi-specific antibody comprising a second variable region that specifically binds to a second target protein, wherein the second target protein is human CD3, wherein the antibody comprises a second heavy chain variable region and a second light chain variable region, and wherein the cancer cell is brought in proximity to a peripheral blood mononuclear cell (PBMC) expressing CD3 by binding of the antibody.

27. The method of claim 26, wherein the PBMC is a T-cell.

28. The method of claim 26, wherein the CDR1, CDR2, and CDR3 of the second heavy chain variable region comprise SEQ ID NO: 79, SEQ ID NO: 80 and SEQ ID NO: 81 and the CDR1, CDR2, and CDR3 of the second light chain variable region comprise SEQ ID NO: 82, SEQ ID NO: 83 and SEQ ID NO: 84.

29. The method of claim 28, wherein the second heavy chain variable region comprises SEQ ID NO: 77 and the second light chain variable region comprises SEQ ID NO:78.

30. The method of claim 28, wherein the antibody comprises SEQ ID NO:85 and SEQ ID NO:86.

31. The method of claim 28, wherein the antibody comprises SEQ ID NO:87 and SEQ ID NO:88.

32. The method of claim 25, wherein the antibody is linked to a cytotoxic agent.

33. The method of claim 32, wherein the cytotoxic agent is a radionucleotide.

34. The method of any one of claims 25-32, wherein the cancer cell is in a human having the cancer cell and the antibody is administered to the human, thereby killing the cancer cell.

35. A chimeric antigen receptor (CAR)-expressing human cell, wherein the CAR comprises the heavy chain variable region and the light chain variable region of any one of claims 1-9.

36. The CAR-expressing human cell of claim 36, wherein the human cell is a T-cell, natural killer cell or a macrophage.

37. A method of detecting a tumor cell in a sample, the method comprising contacting an antibody of any one of claims 1-18 to the sample; and
detecting specific binding of the antibody to the tumor cell.
